(19) United States
(12) Patent Application Publication
(10) Pub. No.: US 2024/0124451 A1
(43) Pub. Date: Apr. 18, 2024

(54) PHTHALAZINE DERIVATIVES USEFUL AS INHIBITORS OF NOD-LIKE RECEPTOR PROTEIN 3

(71) Applicant: Merck Sharp & Dohme LLC, Rahway, NJ (US)

(72) Inventors: Donna A.A.W. Hayes, San Francisco, CA (US); Prabha Karnachi, Hillsborough, NJ (US); Madeleine Eileen Kieffer, Burlingame, CA (US); Kyle S. McClymont, San Francisco, CA (US); Rohan Rajiv Merchant, San Francisco, CA (US); Essam Metwally, Emerald Hills, CA (US); Anilkumar G. Nair, Morganville, NJ (US); Ning Qi, Somerset, NJ (US); Jillian Rose Sanzone, San Francisco, CA (US); Nunzio Sciammetta, Sudbury, MA (US); Emma H. Southgate, Mountain View, MA (US); Zheng Tan, Westfield, NJ (US); Brandon M. Taoka, San Francisco, CA (US)

(73) Assignee: Merck Sharp & Dohme LLC, Rahway, NJ (US)

(21) Appl. No.: 18/471,375

(22) Filed: Sep. 21, 2023

Related U.S. Application Data
(60) Provisional application No. 63/409,432, filed on Sep. 23, 2022.

(51) Int. Cl. C07D 471/04; C07D 413/04; C07D 487/04; C07D 519/00

(52) U.S. Cl. CPC C07D 471/04; C07D 413/04; C07D 487/04; C07D 519/00

(57) ABSTRACT

Novel compounds of the structural formula (I), and the pharmaceutically acceptable salts thereof, are inhibitors of NLRP3 and may be useful in the treatment, prevention, management, amelioration, control and suppression of diseases mediated by NLPR3. The compounds of the present invention may be useful in the treatment, prevention or management of diseases, disorders and conditions mediated by NLRP3 such as, but not limited to, gout, pseudogout, CAPS, NASH fibrosis, heart failure, idiopathic pericarditis, atopic dermatitis, inflammatory bowel disease, Alzheimer's Disease, Parkinson's Disease and traumatic brain injury.

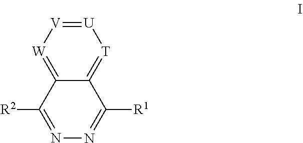

### BACKGROUND OF THE INVENTION

Inflammasomes function as central signalling hubs of the innate immune system. They are multi-protein complexes assembled after activation of intracellular pattern recognition receptors (PRRs) by a variety of pathogen-associated molecular patterns (PAMPs) or danger-associated molecular patterns (DAMPs). It has been shown that inflammasomes can be formed by nucleotide-binding oligomerization domain (NOD)-like receptors (NLRs) and Pyrin and HIN200-domain-containing proteins (Van Opdenbosch N and Lamkanfi M. Immunity, 2019 Jun. 18; 50(6):1352-1364). Inflammasome activation triggers a cascade of events that releases pro-inflammatory cytokines, and promotes an inflammatory form of cell death called pyroptosis induced by the activation of Gasdermin. Pyroptosis is a unique form of inflammatory cell death that leads to the release of not only cytokines but also other intracellular components that promote a broader immune response both of the innate and acquired immune system. Thus, inflammasome activation is a major regulator of the inflammatory cascade.

The (NOD)-like receptor protein 3 (NLRP3) inflammasome is the most well-studied of all the inflammasomes. NLRP3 can be activated by numerous stimuli including environmental crystals, pollutants, host-derived DAMPs and protein aggregates (Tartey S and Kanneganti T D. Immunology, 2019 April; 156(4):329-338). Danger-associated molecular patterns that engage NLRP3 include uric acid and cholesterol crystals that cause gout and atherosclerosis, amyloid-P fibrils that are neurotoxic in Alzheimer's disease, and asbestos particles that cause mesothelioma (Kelley et al., Int J Mol Sci, 2019 Jul. 6; 20(13)). Additionally, NLRP3 is activated by infectious agents, such as Vibrio cholerae, fungal pathogens, such as Aspergillus Jumigatus and Candida albicans, adenoviruses, influenza A virus and SARS-CoV-2 (Tartey and Kanneganti, 2019 (see above); Fung et al. Emerg Microbes Infect, 2020 Mar. 14; 9(1):558-570).

The NLRP3 activation mechanism in humans remains unclear. It has been suggested that the NLRP3 inflammasome requires regulation at both the transcriptional and the post-transcriptional level (Yang Y et al., Cell Death Dis, 2019 Feb. 12; 10(2): 128). The NOD-like receptor protein 3 (NLRP3) is a protein-coding gene that encodes a protein consisting of a N-terminal pyrin domain, a nucleotide-binding site domain (NBD), and a leucine-rich repeat (LRR) motif on the C-terminal (Inoue et al., Immunology, 2013, 139, 11-18; Sharif et al., Nature, 2019 June; 570(7761):338-343).

In response to sterile inflammatory danger signals PAMPs or DAMPs, NLRP3 interacts with the adaptor protein, apoptosis-associated speck-like protein containing a caspase recruitment domain (ASC), and with the protease caspase-1 to form the NLRP3 inflammasome. Upon activation, procaspase-1 undergoes autoproteolysis and cleaves gasdermin D (Gsdmd) to produce the N-terminal Gsdmd molecule that leads to pore-formation in the plasma membrane and results in a lytic form of cell death called pyroptosis. Alternatively, caspase-1 cleaves the pro-inflammatory cytokines pro-IL-Iβ and pro-IL-18 to allow release of its biological active form (Kelley et al., 2019—see above). The NLRP3 inflammasome activation results in the release of the inflammatory cytokines IL-1β (interleukin-Iβ) and IL-18 (interleukin-18), which when dysregulated can lead to a number of diseases.

Dysregulation of the NLRP3 inflammasome or its downstream mediators are associated with numerous immune diseases, inflammatory diseases, auto-immune diseases and auto-inflammatory diseases. Activation of the NLRP3 inflammasome has been linked to the following diseases and disorders: Cryopyrin-associated Periodic Syndromes; sickle cell disease; systemic lupus erythematosus; allodynia; graft versus host disease; hepatic disorders including non-alcoholic steatohepatitis (NASH), chronic liver disease, viral hepatitis, alcoholic steatohepatitis, and alcoholic liver disease; inflammatory bowel diseases including Crohn's disease and ulcerative colitis; inflammatory joint disorders including gout, pseudogout, arthropathy, osteoarthritis, rheumatoid arthritis; additional rheumatic diseases including dermatomyositis, Still's disease, and juvenile idiopathic arthritis. kidney related diseases including hyperoxaluria, lupus nephritis, hypertensive nephropathy, hemodialysis related inflammation, diabetic nephropathy, and diabetic kidney disease and other inflammatory diseases (Miyamae T. Paediatr Drugs, 2012 Apr. 1, 14(2): 109-17; Szabo G and Petrasek J. Nat Rev Gastroenterol Hepatol, 2015 July; 12(7):387-400; Zhen Y and Zhang H. Front Immunol, 2019 Feb. 28; 10:276; Vande Walle L et al., Nature, 2014 Aug. 7; 512(7512):69-73; Knauf et al., Kidney Int, 2013 November; 84(5):895-901; Krishnan et al., Br J Pharmacol, 2016 February; 1 73(4):752-65); Shahzad et al., Kidney Int, 2015 January; 87(1):74-84; Jankovic, et al. J Exp Med. 2013 Sep. 23; 210(10):1899-910.). The onset and progression of neuroinflammation-related disorders, such as brain infection, acute injury, multiple sclerosis, amyotrophic lateral sclerosis and additional neurodegenerative diseases such as Parkinsons and Alzheimer's disease have also been linked to NLRP3 inflammasome activation (Sarkar et al., NPJ Parkinsons Dis, 2017 Oct. 17; 3:30).

Cardiovascular and metabolic disorders such as atherosclerosis, type I and type II diabetes and diabetes complications including nephropathy and retinopathy, peripheral artery disease, acute heart failure and hypertension have been associated to NLRP3 (Ridker et al., CANTOS Trial Group. N Engl J Med, 2017 Sep. 21; 377(12):1119-1131; and Toldo S and Abbate A Nat Rev Cardiol, 2018 April; 15(4):203-214). NLRP3 associated skin diseases include wound healing and scar formation; inflammatory skin diseases such as acne, atopic dermatitis, hidradenitis suppurativa and psoriasis (Kelly et al., Br J Dermatol, 2015 December; 1 73(6)). NLRP3 inflammasome activity has also been linked to respiratory conditions such as asthma, sarcoidosis, acute respiratory distress syndrome, Severe Acute Respiratory Syndrome (SARS) (Nieto-Torres et al., Virology, 2015 November; 485:330-9)); and ocular diseases including age-related macular degeneration (AMD) and diabetic retinopathy (Doyle et al., Nat Med, 2012 May; 18(5):791-8). Cancers linked to NLRP3 include myeloproliferative neoplasms, leukemias, myelodysplastic syndromes, myelofibrosis, lung cancer and colon cancer (Ridker et al., Lancet, 2017 Oct. 21; 390(10105): 1833-1842; Derangere et al., Cell Death Differ. 2014 December; 21(12): 1914-24; Basiorka et al., Lancet Haematol, 2018 September; 5(9): e393-e402, Zhang et al., Hum Immunol, 2018 January; 79(1):57-62).

Immune diseases and inflammatory disorders are typically difficult to diagnose or treat efficiently and effectively. Most treatments include treatment of the symptoms, slowing down disease progression, lifestyle changes and surgery.

There remains a need for inhibitors of NLRP3 to provide new treatments for diseases and disorders associated with NLRP3 inflammasome activation and dysregulation. The compounds of the present invention are useful for the treatment and prevention of diseases, disorders and conditions mediated by formation and propagation of the NLRP3 inflammasome.

NLRP3 inhibitors are disclosed in the following publications: Nat. 2022, 1; Cell. 2021, 184, 1; J. Mol. Biol. 2021, 433, 167308; J. Med. Chem. 2021, 64, 101; Nat. Chem. Biol. 2019, 15, 556; Nat. 2019, 570, 338; Nat. Chem. Biol. 2019, 15, 560; PLOS Biol. 2019, 1; Nat. Med. 2015, 21, 248; Cell. 2014, 156, 1193; Nat. Immunol. 2014, 15, 738; PNAS. 2007, 104, 8041; Nat. 2006, 440, 9; Immunity. 2006, 24, 317. Several patent applications describe NLRP3 inhibitors, including WO 2021/239885, WO 2021/209552, WO 2021/209539, WO 2021/193897, WO 2020/018975, WO 2020/037116, WO 2020/021447, WO 2020/010143, WO 2019/079119, WO 2019/0166621, WO 2019/121691, U.S. Pat. No. 11,319,319, and US 2020/0361898.

### SUMMARY OF THE INVENTION

The present invention relates to novel compounds of structural formula I:

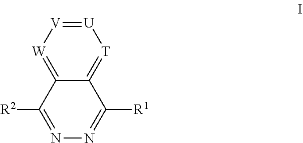

and pharmaceutically acceptable salts thereof.

The compounds of structural formula I, and embodiments thereof, are inhibitors of NOD-like receptor protein 3 (NLRP3) and may be useful in the treatment and prevention of diseases, disorders and conditions mediated by NLRP3 such as, but not limited to, gout, pseudogout (chondrocalcinosis), cryopyrin-associated periodic syndromes (CAPS), NASH, fibrosis, heart failure, idiopathic pericarditis, atopic dermatitis, inflammatory bowel disease, Alzheimer's Disease, Parkinson's Disease and traumatic brain injury.

The present invention also relates to pharmaceutical compositions comprising the compounds of the present invention and a pharmaceutically acceptable carrier.

The present invention also relates to methods for the treatment, management, prevention, alleviation, amelioration, suppression or control of disorders, diseases, and conditions that may be responsive to inhibition of the NLRP3 receptor in a subject in need thereof by administering the compounds and pharmaceutical compositions of the present invention.

The present invention also relates to the use of compounds of the present invention for manufacture of a medicament useful in treating diseases, disorders and conditions that may be responsive to the inhibition of the NLRP3 receptor.

The present invention is also concerned with treatment or prevention of these diseases, disorders and conditions by administering the compounds of the present invention in combination with a therapeutically effective amount of another agent that may be useful to treat the disease, disorder and condition. The invention is further concerned with processes for preparing the compounds of this invention.

### DETAILED DESCRIPTION OF THE INVENTION

The present invention is concerned with novel compounds of structural Formula I:

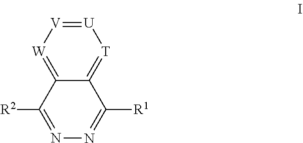

or a pharmaceutically acceptable salt thereof, wherein
T is independently selected from the group:
   1) CR³, and
   2) N,
provided that zero, one or two of T, U, V and W are N;
U is independently selected from the group:
   1) CR⁴, and
   2) N;
V is independently selected from the group:
   1) CR⁵, and
   2) N;
W is independently selected from the group:
   1) CR⁶, and
   2) N;
R¹ is selected from the group:
   1) —C₃₋₁₂cycloalkyl,
   2) —C₃₋₁₂cycloalkenyl, and
   3) —C₂₋₁₂cycloheteroalkyl,
wherein R¹ is unsubstituted or substituted with one to six substituents selected from R^{a};
R² is selected from the group:
   1) aryl, and
   2) heteroaryl,
wherein R² is unsubstituted or substituted with one to five substituents selected from R^{b};
R³ is selected from the group:
   1) hydrogen,
   2) OH,
   3) CN,
   4) CF₃,
   5) CF₂H,
   6) —C₁₋₆alkyl,
   7) —O—C₁₋₆alkyl,
   8) halogen,
   9) —C₃₋₆cycloalkyl,
   10) —C₂₋₆cycloheteroalkyl,
   11) —C₁₋₆alkyl-O—C₁₋₆alkyl,
   12) —(CH₂)ᵣC(O)R^{h},
   13) —(CH₂)ᵣC(O)N(Rⁱ)₂,
   14) —(CH₂)ᵣN(R^{j})C(O)R^{h},
   15) —(CH₂)ᵣN(R^{j})C(O)OR^{h},
   16) —(CH₂)ᵣN(R^{j})C(O)N(Rⁱ)₂,
   17) —(CH₂)ᵣN(R^{j})C(O)N(Rⁱ)₂,
   18) —(CH₂)ᵣN(R^{j})S(O)ₘR^{h},
   19) —(CH₂)ᵣN(R^{j})S(O)ₘN(Rⁱ)₂,
   20) —(CH₂)ᵣN(R^{j})S(O)ₘN(Rⁱ)₂, and
   21) —(CH₂)ᵣN(Rⁱ)₂,
wherein R³ is unsubstituted or substituted with one to five substituents selected from R^{d};
R⁴ is selected from the group:
   1) hydrogen,
   2) OH,
   3) CN,
   4) CF₃,
   5) CF₂H,
   6) —C₁₋₆alkyl,
   7) —O—C₁₋₆alkyl,
   8) halogen,
   9) —C₃₋₆cycloalkyl,
   10) —C₂₋₆cycloheteroalkyl,
   11) —C₁₋₆alkyl-O—C₁₋₆alkyl,
   12) —(CH₂)ₛC(O)R^{h},
   13) —(CH₂)ₛC(O)N(Rⁱ)₂,
   14) —(CH₂)ₛN(R^{j})C(O)R^{h},
   15) —(CH₂)ₛN(R^{j})C(O)OR^{h},
   16) —(CH₂)ₛN(R^{j})C(O)N(Rⁱ)₂,
   17) —(CH₂)ₛN(R^{j})C(O)N(Rⁱ)₂,
   18) —(CH₂)ₛN(R^{j})S(O)ₘR^{h},
   19) —(CH₂)ₛN(R^{j})S(O)ₘN(Rⁱ)₂,
   20) —(CH₂)ₛN(R^{j})S(O)ₘN(Rⁱ)₂, and
   21) —(CH₂)ₛN(Rⁱ)₂,
wherein R⁴ is unsubstituted or substituted with one to five substituents selected from R^{e};
R⁵ is selected from the group:
   1) hydrogen,
   2) OH,
   3) CN,
   4) CF₃,
   5) CF₂H,
   6) —C₁₋₆alkyl,
   7) —O—C₁₋₆alkyl,
   8) halogen,
   9) —C₃₋₆cycloalkyl,
   10) —C₂₋₆cycloheteroalkyl,
   11) —C₁₋₆alkyl-O—C₁₋₆alkyl,
   12) —(CH₂)ₜC(O)R^{h},
   13) —(CH₂)ₜC(O)N(Rⁱ)₂,
   14) —(CH₂)ₜN(R^{j})C(O)R^{h},
   15) —(CH₂)ₜN(R^{j})C(O)OR^{h},
   16) —(CH₂)ₜN(R^{j})C(O)N(Rⁱ)₂,
   17) —(CH₂)ₜN(R^{j})C(O)N(Rⁱ)₂,
   18) —(CH₂)ₜN(R^{j})S(O)ₘR^{h},
   19) —(CH₂)ₜN(R^{j})S(O)ₘN(Rⁱ)₂,
   20) —(CH₂)ₜN(R^{j})S(O)ₘN(Rⁱ)₂, and
   21) —(CH₂)ₜN(Rⁱ)₂,
wherein R⁵ is unsubstituted or substituted with one to five substituents selected from R^{f};
R⁶ is selected from the group:
   1) hydrogen,
   2) OH,
   3) CN,
   4) CF₃,
   5) CF₂H,
   6) —C₁₋₆alkyl,
   7) —O—C₁₋₆alkyl,
   8) halogen,
   9) —C₃₋₆cycloalkyl,
   10) —C₂₋₆cycloheteroalkyl,
   11) —C₁₋₆alkyl-O—C₁₋₆alkyl,
   12) —(CH₂)ᵤC(O)R^{h},
   13) —(CH₂)ᵤC(O)N(Rⁱ)₂,
   14) —(CH₂)ᵤN(R^{j})C(O)R^{h},
   15) —(CH₂)ᵤN(R^{j})C(O)OR^{h},
   16) —(CH₂)ᵤN(R^{j})C(O)N(Rⁱ)₂,
   17) —(CH₂)ᵤN(R^{j})C(O)N(Rⁱ)₂,
   18) —(CH₂)ᵤN(R^{j})S(O)ₘR^{h},
   19) —(CH₂)ᵤN(R^{j})S(O)ₘN(Rⁱ)₂,
   20) —(CH₂)ᵤN(R^{j})S(O)ₘN(Rⁱ)₂, and
   21) —(CH₂)ᵤN(Rⁱ)₂,
wherein R⁶ is unsubstituted or substituted with one to five substituents selected from R^{g};
each R^{a} is independently selected from the group:
   1) CN,
   2) oxo,
   3) —OH,
   4) halogen,
   5) —C₁₋₆alkyl,
   6) —O—C₁₋₆alkyl,
   7) —C₂₋₆alkenyl,
   8) —C₂₋₆alkynyl,
   9) —C₃₋₆cycloalkyl,
   10) —C₂₋₆cycloheteroalkyl,
   11) aryl,
   12) heteroaryl,
   13) —C(O)C₁₋₆alkyl,
   14) —C₁₋₆alkyl-aryl,
   15) —C₁₋₆alkyl-heteroaryl,
   16) —C₁₋₆alkyl-C₃₋₆cycloalkyl,
   17) —C₁₋₆alkyl-C₂₋₆cycloheteroalkyl,
   18) —C₂₋₆alkenyl-C₃₋₆cycloalkyl,
   19) —C₂₋₆alkenyl-C₂₋₆cycloheteroalkyl,
   20) —C₂₋₆alkenyl-aryl,
   21) —C₂₋₆alkenyl-heteroaryl,
   22) —C₂₋₆alkynyl-C₃₋₆cycloalkyl,
   23) —C₂₋₆alkynylC₂₋₆cycloheteroalkyl,
   24) —C₂₋₆alkynyl-aryl,
   25) —C₂₋₆alkynyl-heteroaryl,
   26) —(CH₂)ₚ—O—C₁₋₆alkyl,
   27) —(CH₂)ₚ—O—C₂₋₆alkenyl,
   28) —(CH₂)ₚ—O—C₂₋₆alkynyl,
   29) —(CH₂)ₚ—O—C₃₋₆cycloalkyl,
   30) —(CH₂)ₚ—O—C₂₋₆cycloheteroalkyl,
   31) —(CH₂)ₚ—O-aryl,
   32) —(CH₂)ₚ—O-heteroaryl,
   33) —OC₁₋₆alkyl-C₃₋₆cycloalkyl,
   34) —OC₁₋₆alkyl-C₂₋₆cycloheteroalkyl,
   35) —OC₁₋₆alkyl-aryl,
   36) —OC₁₋₆alkyl-heteroaryl,
   37) —(CH₂)ₚ—S(O)ᵣR^{k},
   38) —(CH₂)ₚ—S(O)N(R^{L})₂, and
   39) —(CH₂)ₚ—N(R^{L})₂,
wherein each R^{a} is unsubstituted or substituted with one to six substituents selected from halogen, CF₃, OH, C₁₋₆alkyl, and —OC₁₋₆alkyl;
each R^{b} is independently selected from the group:
   1) CN,
   2) —OH,
   3) oxo,
   4) halogen,
   5) —C₁₋₆alkyl,
   6) —O—C₁₋₆alkyl,
   7) —C₃₋₆cycloalkyl,
   8) —C₂₋₆cycloheteroalkyl,
   9) aryl,
   10) heteroaryl,
   11) —C₁₋₆alkyl-aryl,
   12) —C₁₋₆alkyl-heteroaryl,
   13) —C₁₋₆alkyl-C₃₋₆cycloalkyl,
   14) —C₁₋₆alkyl-C₂₋₆cycloheteroalkyl,
   15) —(CH₂)_{q}—O—C₁₋₆alkyl,
   16) —(CH₂)_{q}—O—C₃₋₆cycloalkyl,
   17) —(CH₂)_{q}—O—C₂₋₆cycloheteroalkyl,
   18) —(CH₂)_{q}—O-aryl,
   19) —(CH₂)_{q}—O-heteroaryl,
   20) —OC₁₋₆alkyl-C₃₋₆cycloalkyl,
   21) —OC₁₋₆alkyl-C₂₋₆cycloheteroalkyl,
   22) —OC₁₋₆alkyl-aryl,
   23) —OC₁₋₆alkyl-heteroaryl,
   24) —(CH₂)_{q}—S(O)ᵣR^{m},
   25) —(CH₂)_{q}N(Rⁿ)₂,
   26) —C(O)R^{o}, and
   27) —C(O)NRⁿ,
wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl;
each R^{d} is independently selected from the group:
   1) CF₃,
   2) halogen, and
   3) —C₁₋₆alkyl,
wherein alkyl is unsubstituted or substituted with one to three substituents selected from: CF₃, halogen, OH and —OC₁₋₆alkyl;
each R^{e} is independently selected from the group:
   1) CF₃,
   2) halogen, and
   3) —C₁₋₆alkyl,
wherein alkyl is unsubstituted or substituted with one to three substituents selected from: CF₃, halogen, OH and —OC₁₋₆alkyl;
each R^{f} is independently selected from the group:
   1) CF₃,
   2) halogen, and
   3) —C₁₋₆alkyl,
wherein alkyl is unsubstituted or substituted with one to three substituents selected from: CF₃, halogen, OH and —OC₁₋₆alkyl;
each R^{g} is independently selected from the group:
   1) CF₃,
   2) halogen, and
   3) —C₁₋₆alkyl,
wherein alkyl is unsubstituted or substituted with one to three substituents selected from: CF₃, halogen, OH and —OC₁₋₆alkyl;
each R^{h} is independently selected from the group:
   1) hydrogen,
   2) —C₁₋₆alkyl,
   3) —C₃₋₆cycloalkyl, and
   4) —C₂₋₆cycloheteroalkyl,
wherein alkyl, cycloalkyl and cycloheteroalkyl is unsubstituted or substituted with one to three substituents selected from: —CF₃, halogen, OH and —OC₁₋₆alkyl;
each Rⁱ is independently selected from the group:
   1) hydrogen,
   2) —C₁₋₆alkyl,
   3) —C₃₋₆cycloalkyl, and
   4) —C₂₋₆cycloheteroalkyl,
wherein alkyl, cycloalkyl and cycloheteroalkyl is unsubstituted or substituted with one to three substituents selected from: CF₃, halogen, OH and —OC₁₋₆alkyl;
each R^{j} is independently selected from the group:
   1) hydrogen,
   2) —C₁₋₆alkyl,
   3) —C₃₋₆cycloalkyl, and
   4) —C₂₋₆cycloheteroalkyl,
wherein alkyl, cycloalkyl and cycloheteroalkyl is unsubstituted or substituted with one to three substituents selected from: CF₃, halogen, OH and —OC₁₋₆alkyl;
each R^{k} is independently selected from the group:
   1) —C₁₋₆alkyl,
   2) —C₃₋₆cycloalkyl, and
   3) —C₂₋₆cycloheteroalkyl,
wherein alkyl, cycloalkyl, and cycloheteroalkyl is unsubstituted or substituted with one to three substituents selected from: —CF₃, halogen, OH and —OC₁₋₆alkyl;
each R^{L} is independently selected from the group:
   1) hydrogen,
   2) —C₁₋₆alkyl,
   3) —C₃₋₆cycloalkyl, and
   4) —C₂₋₆cycloheteroalkyl,
wherein alkyl, cycloalkyl, and cycloheteroalkyl is unsubstituted or substituted with one to three substituents selected from: —CF₃, halogen, OH and —OC₁₋₆alkyl;
each R^{m} is independently selected from the group:
   1) —C₁₋₆alkyl,
   2) —C₃₋₆cycloalkyl, and
   3) —C₂₋₆cycloheteroalkyl,
wherein alkyl, cycloalkyl and cycloheteroalkyl is unsubstituted or substituted with one to three substituents selected from: —CF₃, halogen, OH and —OC₁₋₆alkyl;
each Rⁿ is independently selected from the group:
   1) hydrogen,
   2) C₁₋₆alkyl,
   3) —C₃₋₆cycloalkyl, and
   4) —C₂₋₆cycloheteroalkyl,
wherein alkyl, cycloalkyl, and cycloheteroalkyl is unsubstituted or substituted with one to three substituents selected from: —CF₃, halogen, OH and —OC₁₋₆alkyl;
each R^{o} is independently selected from the group:
   1) OH,
   2) —C₁₋₆alkyl,
   3) —C₃₋₆cycloalkyl, and
   4) —C₂₋₆cycloheteroalkyl,
wherein alkyl, cycloalkyl and cycloheteroalkyl is unsubstituted or substituted with one to three substituents selected from —CF₃, halogen, OH and —OC₁₋₆alkyl;
   r is 0, 1, 2, 3, 4, 5, or 6;
   s is 0, 1, 2, 3, 4, 5, or 6;
   t is 0, 1, 2, 3, 4, 5, or 6;
   u is 0, 1, 2, 3, 4, 5, or 6;
   p is 0, 1, 2, 3, 4, 5, or 6; and
   q is 0, 1, 2, 3, 4, 5, or 6.

The invention has numerous embodiments, which are summarized below. The invention includes the compounds as shown, and also includes individual diastereoisomers, enantiomers, and epimers of the compounds, and mixtures of diastereoisomers and/or enantiomers thereof including racemic mixtures.

In one embodiment of the present invention, T is independently selected from the group: CR³ and N, provided that zero, one or two of T, U, V and W are N. In another embodiment of the present invention, T is CR³, provided that zero, one or two of T, U, V and W are N. In another embodiment of the present invention, T is CR³. In another embodiment of the present invention, T is N, provided that zero, one or two of U, V and W are N. In another embodiment of the present invention, T is N.

In another embodiment of the present invention, T is independently selected from the group: CR³ and N, provided that one or two of T, U, V and W are N.

In another embodiment of the present invention, U is selected from the group: CR⁴ and N.

In another embodiment of the present invention, U is CR⁴. In another embodiment of the present invention, U is N.

In another embodiment of the present invention, V is selected from the group: CR⁵ and N.

In another embodiment of the present invention, V is CR⁵. In another embodiment of the present invention, V is N.

In another embodiment of the present invention, W is selected from the group: CR⁶ and N. In another embodiment of the present invention, W is CR⁶. In another embodiment of the present invention, W is N.

In another embodiment of the present invention, T is CR³; U is CR⁴; V is CR⁵; and W is CR⁶. In another embodiment of the present invention, T is N; U is CR⁴; V is CR⁵; and W is CR⁶. In another embodiment of the present invention, T is CR³; U is CR⁴; V is N; and W is CR⁶. In another embodiment of the present invention, T is CR³; U is CR⁴; V is CR⁵; and W is N. In another embodiment of the present invention, T and U are N; V is CR⁵; and W is CR⁶. In another embodiment of the present invention, T and V are N; U is CR⁴; and W is CR⁶. In another embodiment of the present invention, T and W are N; U is CR⁴; and V is CR⁵. In another embodiment of the present invention, U and V are N; T is CR³; and W is CR⁶. In another embodiment of the present invention, U and W are N; T is CR³; and V is CR⁵. In another embodiment of the present invention, V and W are N; T is CR³; and U is CR⁴. In another embodiment of the present invention, T is CR³; U is N; V is CR⁵; and W is CR⁶.

In another embodiment of the present invention, R¹ is selected from the group: —C₃₋₁₂cycloalkyl, —C₃₋₁₂cycloalkenyl, and —C₂₋₁₂cycloheteroalkyl, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment of the present invention, R¹ is —C₃₋₁₂cycloalkenyl, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment of the present invention, R¹ is selected from the group: —C₃₋₁₂cycloalkyl and —C₂₋₁₂cycloheteroalkyl, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment of the present invention, R¹ is —C₃₋₁₂cycloalkyl, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment of the present invention, R¹ is C₂₋₁₂cycloheteroalkyl, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment, R¹ is selected from the group: morpholine, thiomorpholine, piperidine, piperazine, pyrrolidine, tetrahydropyran, octahydro-1H-pyrrolo[2,3-c]pyridine, 3-azabicyclo[3.1.0]hexane, 5-azaspiro[2.4]heptane, 1-oxa-7-azaspiro[4.4]nonane, 1-oxa-8-azaspiro[4.5]decane, 3-oxa-1,8-diazaspiro[4.5]decane, 2,8-diazaspiro[4.5]decane, 1-oxa-3,8-diazaspiro[4.5]decane, 2-oxa-8-azaspiro[4.5]decane, 1,8-diazaspiro[4.5]decane, and 1-oxa-4,9-diazaspiro[5.5]undecane, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment, R¹ is selected from the group: morpholine, thiomorpholine, piperidine, pyrrolidine, tetrahydropyran, octahydro-1H-pyrrolo[2,3-c]pyridine, 3-azabicyclo[3.1.0]hexane, 5-azaspiro[2.4]heptane, 1-oxa-7-azaspiro[4.4]nonane, 1-oxa-8-azaspiro[4.5]decane, 3-oxa-1,8-diazaspiro[4.5]decane, 2,8-diazaspiro[4.5]decane, 1-oxa-3,8-diazaspiro[4.5]decane, 2-oxa-8-azaspiro[4.5]decane, 1,8-diazaspiro[4.5]decane, and 1-oxa-4,9-diazaspiro[5.5]undecane, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment of the present invention, R¹ is selected from the group: morpholine, piperidine, pyrrolidine, tetrahydropyran, octahydro-1H-pyrrolo[2,3-c]pyridine, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment of the present invention, R¹ is selected from the group: morpholine, pyrrolidine, tetrahydropyran, octahydro-1H-pyrrolo[2,3-c]pyridine, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment of the present invention, R¹ is pyrrolidine, wherein R¹ is unsubstituted or substituted with one to six substituents selected from R^{a}. In a class of this embodiment, R¹ is unsubstituted or substituted with one to five substituents selected from R^{a}. In another class of this embodiment, R¹ is unsubstituted or substituted with one to four substituents selected from R^{a}.

In another embodiment of the present invention, R² is selected from the group: aryl and heteroaryl, wherein R² is unsubstituted or substituted with one to five substituents selected from R^{b}.

In a class of this embodiment, R² is unsubstituted or substituted with one to four substituents selected from R^{b}. In another class of this embodiment, R² is unsubstituted or substituted with one to three substituents selected from R^{b}. In another class of this embodiment, R² is unsubstituted or substituted with one to two substituents selected from R^{b}.

In another embodiment, R² is heteroaryl, wherein R² is unsubstituted or substituted with one to five substituents selected from R^{b}. In another class of this embodiment, R² is unsubstituted or substituted with one to four substituents selected from R^{b}. In another class of this embodiment, R² is unsubstituted or substituted with one to three substituents selected from R^{b}. In another class of this embodiment, R² is unsubstituted or substituted with one to two substituents selected from R^{b}.

In another embodiment, R² is pyridine or benzofuran, wherein R² is unsubstituted or substituted with one to five substituents selected from R^{b}. In a class of this embodiment, R² is unsubstituted or substituted with one to four substituents selected from R^{b}. In another class of this embodiment, R² is unsubstituted or substituted with one to three substituents selected from R^{b}. In another class of this embodiment, R² is unsubstituted or substituted with one to two substituents selected from R^{b}.

In another embodiment, R² is pyridine, wherein R² is unsubstituted or substituted with one to five substituents selected from R^{b}. In a subclass of this class, R² is unsubstituted or substituted with one to four substituents selected from R^{b}. In another subclass of this class, R² is unsubstituted or substituted with one to three substituents selected from R^{b}. In another subclass of this class, R² is unsubstituted or substituted with one to two substituents selected from R^{b}.

In another embodiment, R² is benzofuran, wherein R² is unsubstituted or substituted with one to five substituents selected from R^{b}. In a subclass of this class, R² is unsubstituted or substituted with one to four substituents selected from R^{b}. In another subclass of this class, R² is unsubstituted or substituted with one to three substituents selected from R^{b}. In another subclass of this class, R² is unsubstituted or substituted with one to two substituents selected from R^{b}.

In another embodiment, R² is aryl, wherein R² is unsubstituted or substituted with one to five substituents selected from R^{b}. In a class of this embodiment, R² is unsubstituted or substituted with one to four substituents selected from R^{b}. In another class of this embodiment, R² is unsubstituted or substituted with one to three substituents selected from R^{b}. In another class of this embodiment, R² is unsubstituted or substituted with one to two substituents selected from R^{b}. In another class of this embodiment, R² is phenyl, wherein R² is unsubstituted or substituted with one to five substituents selected from R^{b}. In a subclass of this class, R² is unsubstituted or substituted with one to four substituents selected from R^{b}. In another subclass of this class, R² is unsubstituted or substituted with one to three substituents selected from R^{b}. In another subclass of this class, R² is unsubstituted or substituted with one to two substituents selected from R^{b}.

In another embodiment of the present invention, R³ is selected from the group: hydrogen, OH, CN, CF₃, CF₂H, —C₁₋₆alkyl, —O—C₁₋₆alkyl, halogen, —C₃₋₆cycloalkyl, —C₂₋₆cycloheteroalkyl, —C₁₋₆alkyl-O—C₁₋₆alkyl, —(CH₂)ᵣC(O)R^{h}, —(CH₂)ᵣC(O)N(Rⁱ)₂, —(CH₂)ᵣN(R^{j})C(O)R^{h}, —(CH₂)ᵣN(R^{j})C(O)OR^{h}, —(CH₂)ᵣN(R^{j})C(O)N(Rⁱ)₂, —(CH₂)ᵣN(R^{j})C(O)N(Rⁱ)₂, —(CH₂)ᵣN(R^{j})S(O)ₘR^{h}, —(CH₂)ᵣN(R^{j})S(O)ₘN(Rⁱ)₂, —(CH₂)ᵣN(R^{j})S(O)ₘN(Rⁱ)₂, and —(CH₂)ᵣN(Rⁱ)₂, wherein R³ is unsubstituted or substituted with one to five substituents selected from R^{d}.

In another embodiment of the present invention, R³ is selected from the group: hydrogen, OH, CN, CF₃, CF₂H, —C₁₋₆alkyl, —O—C₁₋₆alkyl, halogen, and —C₁₋₆alkyl-O—C₁₋₆alkyl, wherein R³ is unsubstituted or substituted with one to five substituents selected from R^{d}.

In another embodiment of the present invention, R³ is selected from the group: hydrogen, OH, CN, CF₃, CF₂H, —C₁₋₆alkyl, —O—C₁₋₆alkyl, and halogen, wherein R³ is unsubstituted or substituted with one to five substituents selected from R^{d}.

In another embodiment of the present invention, R³ is selected from the group: hydrogen, OH, CN, CF₃, CF₂H, —C₁₋₆alkyl, and —O—C₁₋₆alkyl, wherein R³ is unsubstituted or substituted with one to five substituents selected from R^{d}.

In another embodiment of the present invention, R³ is selected from the group: hydrogen, OH, CF₃, CF₂H, —C₁₋₆alkyl, and —O—C₁₋₆alkyl, wherein R³ is unsubstituted or substituted with one to five substituents selected from R^{d}.

In another embodiment of the present invention, R³ is selected from the group: hydrogen, —C₁₋₆alkyl, and —O—C₁₋₆alkyl, wherein R³ is unsubstituted or substituted with one to five substituents selected from R^{d}. In a class of this embodiment, R³ is selected from the group: hydrogen, —CH₃, and —OCH₃.

In another embodiment of the present invention, R³ is selected from the group: hydrogen, OH, CN, CF₃, CF₂H, and —C₁₋₆alkyl, wherein R³ is unsubstituted or substituted with one to five substituents selected from R^{d}.

In another embodiment of the present invention, R³ is selected from the group: hydrogen, OH, CN, CF₃, CF₂H, and —CH₃, wherein R³ is unsubstituted or substituted with one to five substituents selected from R^{d}.

In another embodiment of the present invention, R³ is selected from the group: hydrogen, OH, CF₃, CF₂H, and —CH₃, wherein R³ is unsubstituted or substituted with one to five substituents selected from R^{d}.

In another embodiment of the present invention, R³ is selected from the group: hydrogen, CF₃, CF₂H, and —C₁₋₆alkyl, wherein R³ is unsubstituted or substituted with one to five substituents selected from R^{d}.

In another embodiment of the present invention, R³ is selected from the group: hydrogen, CF₃, CF₂H, and —CH₃, wherein R³ is unsubstituted or substituted with one to five substituents selected from R^{d}.

In another embodiment of the present invention, R³ is selected from the group: hydrogen, CF₃, and —C₁₋₆alkyl, wherein R³ is unsubstituted or substituted with one to five substituents selected from R^{d}.

In another embodiment of the present invention, R³ is selected from the group: hydrogen and —C₁₋₆alkyl, wherein R³ is unsubstituted or substituted with one to five substituents selected from R^{d}. In a class of this embodiment, R³ is hydrogen or CH₃.

In another embodiment of the present invention, R³ is —C₁₋₆alkyl, wherein R³ is unsubstituted or substituted with one to five substituents selected from R^{d}. In a class of this embodiment, R³ is —CH₃. In another embodiment of the present invention, R³ is hydrogen. In another embodiment of the present invention, R³ is —CF₃.

In another embodiment of the present invention, R⁴ is selected from the group: hydrogen, OH, CN, CF₃, CF₂H, —C₁₋₆alkyl, —O—C₁₋₆alkyl, halogen, —C₃₋₆cycloalkyl, —C₂₋₆cycloheteroalkyl, —C₁₋₆alkyl-O—C₁₋₆alkyl, —(CH₂)ₛC(O)R^{h}, —(CH₂)ₛC(O)N(Rⁱ)₂, —(CH₂)ₛN(R^{j})C(O)R^{h}, —(CH₂)ₛN(R^{j})C(O)OR^{h}, —(CH₂)ₛN(R^{j})C(O)N(Rⁱ)₂, —(CH₂)ₛN(R^{j})C(O)N(Rⁱ)₂, —(CH₂)ₛN(R^{j})S(O)ₘR^{h}, —(CH₂)ₛN(R^{j})S(O)ₘN(Rⁱ)₂, —(CH₂)ₛN(R^{j})S(O)ₘN(Rⁱ)₂, and —(CH₂)ₛN(Rⁱ)₂, wherein R⁴ is unsubstituted or substituted with one to five substituents selected from R^{e}.

In another embodiment of the present invention, R⁴ is selected from the group: hydrogen, OH, CN, CF₃, CF₂H, —C₁₋₆alkyl, —O—C₁₋₆alkyl, halogen, —C₃₋₆cycloalkyl, —C₂₋₆cycloheteroalkyl, and —C₁₋₆alkyl-O—C₁₋₆alkyl, wherein R⁴ is unsubstituted or substituted with one to five substituents selected from R^{e}.

In another embodiment of the present invention, R⁴ is selected from the group: hydrogen, OH, CN, CF₃, CF₂H, —C₁₋₆alkyl, —O—C₁₋₆alkyl, halogen, and —C₁₋₆alkyl-O—C₁₋₆alkyl, wherein R⁴ is unsubstituted or substituted with one to five substituents selected from R^{e}.

In another embodiment of the present invention, R⁴ is selected from the group: hydrogen, OH, CN, CF₃, CF₂H, —C₁₋₆alkyl, —O—C₁₋₆alkyl, and halogen, wherein R⁴ is unsubstituted or substituted with one to five substituents selected from R^{e}.

In another embodiment of the present invention, R⁴ is selected from the group: hydrogen, CN, CF₃, CF₂H, —C₁₋₆alkyl, and halogen, wherein R⁴ is unsubstituted or substituted with one to five substituents selected from R^{e}.

In another embodiment of the present invention, R⁴ is selected from the group: hydrogen, CF₃, CF₂H, —C₁₋₆alkyl, and halogen, wherein R⁴ is unsubstituted or substituted with one to five substituents selected from R^{e}.

In another embodiment of the present invention, R⁴ is selected from the group: hydrogen, CF₃, and —C₁₋₆alkyl, wherein R⁴ is unsubstituted or substituted with one to five substituents selected from R^{e}.

In another embodiment of the present invention, R⁴ is selected from the group: hydrogen and —C₁₋₆alkyl, wherein R⁴ is unsubstituted or substituted with one to five substituents selected from R^{e}. In a class of this embodiment of the present invention, R⁴ is selected from the group: hydrogen, —CH₃, —CF₃, and —CHF₂. In another class of this embodiment of the present invention, R⁴ is selected from the group: hydrogen and —CH₃. wherein R⁴ is unsubstituted or substituted with one to five substituents selected from R^{e}.

In another embodiment of the present invention, R⁴ is hydrogen.

In another embodiment of the present invention, R⁴ is —C₁₋₆alkyl, wherein R⁴ is unsubstituted or substituted with one to five substituents selected from R^{e}. In a class of this embodiment, R⁴ is —CH₃.

In another embodiment of the present invention, R⁵ is selected from the group: hydrogen, OH, CN, CF₃, CF₂H, —C₁₋₆alkyl, —O—C₁₋₆alkyl, halogen, —C₃₋₆cycloalkyl, —C₂₋₆cycloheteroalkyl, —C₁₋₆alkyl-O—C₁₋₆alkyl, —(CH₂)ₜC(O)R^{h}, —(CH₂)ₜC(O)N(Rⁱ)₂, —(CH₂)ₜN(R^{j})C(O)R^{h}, —(CH₂)ₜN(R^{j})C(O)OR^{h}, —(CH₂)ₜN(R^{j})C(O)N(Rⁱ)₂, —(CH₂)ₜN(R^{j})C(O)N(Rⁱ)₂, —(CH₂)ₜN(R^{j})S(O)ₘR^{h}, —(CH₂)ₜN(R^{j})S(O)ₘN(Rⁱ)₂, —(CH₂)ₜN(R^{j})S(O)ₘN(Rⁱ)₂, and —(CH₂)ₜN(Rⁱ)₂, wherein R⁵ is unsubstituted or substituted with one to five substituents selected from R^{f}.

In another embodiment of the present invention, R⁵ is selected from the group: hydrogen, OH, CN, CF₃, CF₂H, —C₁₋₆alkyl, —O—C₁₋₆alkyl, halogen, —C₃₋₆cycloalkyl, —C₂₋₆cycloheteroalkyl, and —C₁₋₆alkyl-O—C₁₋₆alkyl, wherein R⁵ is unsubstituted or substituted with one to five substituents selected from R^{f}.

In another embodiment of the present invention, R⁵ is selected from the group: hydrogen, OH, CN, CF₃, CF₂H, —C₁₋₆alkyl, —O—C₁₋₆alkyl, halogen, and —C₁₋₆alkyl-O—C₁₋₆alkyl, wherein R⁵ is unsubstituted or substituted with one to five substituents selected from R^{f}. In another embodiment of the present invention, R⁵ is selected from the group: hydrogen, OH, CN, CF₃, CF₂H, —C₁₋₆alkyl, —O—C₁₋₆alkyl, and halogen, wherein R⁵ is unsubstituted or substituted with one to five substituents selected from R^{f}. In another embodiment of the present invention, R⁵ is selected from the group: hydrogen, CN, CF₃, CF₂H, —C₁₋₆alkyl, and halogen, wherein R⁵ is unsubstituted or substituted with one to five substituents selected from R^{f}. In another embodiment of the present invention, R⁵ is selected from the group: hydrogen, CF₃, CF₂H, —C₁₋₆alkyl, and halogen, wherein R⁵ is unsubstituted or substituted with one to five substituents selected from R^{f}.

In another embodiment of the present invention, R⁵ is selected from the group: hydrogen, —C₁₋₆alkyl, and halogen, wherein R⁵ is unsubstituted or substituted with one to five substituents selected from R^{f}. In a class of this embodiment, R⁵ is selected from the group: hydrogen, —CH₃, CHF₂, and F. In another class of this embodiment, R⁵ is selected from the group: hydrogen, —CH₃, and CHF₂. In another class of this embodiment, R⁵ is selected from the group: hydrogen, —CH₃, and F. In another embodiment of the present invention, R⁵ is selected from the group: hydrogen and —C₁₋₆alkyl, wherein R⁵ is unsubstituted or substituted with one to five substituents selected from R^{f}. In a class of this embodiment, R⁵ is selected from the group: hydrogen, CHF₂ and —CH₃. In another embodiment of the present invention, R⁵ is —C₁₋₆alkyl, wherein R⁵ is unsubstituted or substituted with one to five substituents selected from R^{f}. In a class of this embodiment, R⁵ is —CH₃. In another embodiment of the present invention, R⁵ is hydrogen. In another embodiment, R⁵ is F. In another embodiment, R⁵ is CHF₂.

In another embodiment of the present invention, R⁶ is selected from the group: hydrogen, OH, CN, CF₃, CF₂H, —C₁₋₆alkyl, —O—C₁₋₆alkyl, halogen, —C₃₋₆cycloalkyl, —C₂₋₆cycloheteroalkyl, —C₁₋₆alkyl-O—C₁₋₆alkyl, —(CH₂)ᵤC(O)R^{h}, —(CH₂)ᵤC(O)N(Rⁱ)₂, —(CH₂)ᵤN(R^{j})C(O)R^{h}, —(CH₂)ᵤN(R^{j})C(O)OR^{h}, —(CH₂)ᵤN(R^{j})C(O)N(Rⁱ)₂, —(CH₂)ᵤN(R^{j})C(O)N(Rⁱ)₂, —(CH₂)ᵤN(R^{j})S(O)ₘR^{h}, —(CH₂)ᵤN(R^{j})S(O)ₘN(Rⁱ)₂, —(CH₂)ᵤN(R^{j})S(O)ₘN(Rⁱ)₂, and —(CH₂)ᵤN(Rⁱ)₂, wherein R⁶ is unsubstituted or substituted with one to five substituents selected from R^{g}.

In another embodiment of the present invention, R⁶ is selected from the group: hydrogen, OH, CN, CF₃, CF₂H, —C₁₋₆alkyl, —O—C₁₋₆alkyl, halogen, —C₃₋₆cycloalkyl, —C₂₋₆cycloheteroalkyl, and —C₁₋₆alkyl-O—C₁₋₆alkyl, wherein R⁶ is unsubstituted or substituted with one to five substituents selected from R^{g}.

In another embodiment of the present invention, R⁶ is selected from the group: hydrogen, OH, CN, CF₃, CF₂H, —C₁₋₆alkyl, —O—C₁₋₆alkyl, halogen, and —C₁₋₆alkyl-O—C₁₋₆alkyl, wherein R⁶ is unsubstituted or substituted with one to five substituents selected from R^{g}.

In another embodiment of the present invention, R⁶ is selected from the group: hydrogen, CN, CF₃, CF₂H, —C₁₋₆alkyl, —O—C₁₋₆alkyl, and halogen, wherein R⁶ is unsubstituted or substituted with one to five substituents selected from R^{g}.

In another embodiment of the present invention, R⁶ is selected from the group: hydrogen, CF₃, CF₂H, —C₁₋₆alkyl, and —O—C₁₋₆alkyl, wherein R⁶ is unsubstituted or substituted with one to five substituents selected from R^{g}.

In another embodiment of the present invention, R⁶ is selected from the group: hydrogen, CF₃, —C₁₋₆alkyl, and —O—C₁₋₆alkyl, wherein R⁶ is unsubstituted or substituted with one to five substituents selected from R^{g}. In a class of this embodiment, R⁶ is selected from the group: hydrogen, CF₃, —CH₃, and —OCH₃.

In another embodiment of the present invention, R⁶ is selected from the group: hydrogen, —C₁₋₆alkyl, and —O—C₁₋₆alkyl, wherein R⁶ is unsubstituted or substituted with one to five substituents selected from R^{g}. In a class of this embodiment, R⁶ is selected from the group: hydrogen, —CH₃, and —OCH₃.

In another embodiment of the present invention, R⁶ is selected from the group: hydrogen and —C₁₋₆alkyl, wherein R⁶ is unsubstituted or substituted with one to five substituents selected from R^{g}. In a class of this embodiment, R⁶ is selected from the group: hydrogen and CH₃. In another embodiment of the present invention, R⁶ is —C₁₋₆alkyl, wherein R⁶ is unsubstituted or substituted with one to five substituents selected from R^{g}. In a class of this embodiment, R⁶ is —CH₃. In another embodiment of the present invention, R⁶ is hydrogen.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynylC₂₋₆cycloheteroalkyl, —C₂₋₆alkynyl-aryl, —C₂₋₆alkynyl-heteroaryl, —(CH₂)ₚ—O—C₁₋₆alkyl, —(CH₂)ₚ—O—C₂₋₆alkenyl, —(CH₂)ₚ—O—C₂₋₆alkynyl, —(CH₂)ₚ—O—C₃₋₆cycloalkyl, —(CH₂)ₚ—O—C₂₋₆cycloheteroalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆cycloheteroalkyl, —OC₁₋₆alkyl-aryl, —OC₁₋₆alkyl-heteroaryl, —(CH₂)ₚ—S(O)ᵣR^{k}, —(CH₂)ₚ—S(O)N(R^{L})₂, and —(CH₂)ₚ—N(R^{L})₂, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynylC₂₋₆cycloheteroalkyl, —C₂₋₆alkynyl-aryl, —C₂₋₆alkynyl-heteroaryl, —(CH₂)ₚ—O—C₁₋₆alkyl, —(CH₂)ₚ—O—C₂₋₆alkenyl, —(CH₂)ₚ—O—C₂₋₆alkynyl, —(CH₂)ₚ—O—C₃₋₆cycloalkyl, —(CH₂)ₚ—O—C₂₋₆cycloheteroalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆cycloheteroalkyl, —OC₁₋₆alkyl-aryl, —OC₁₋₆alkyl-heteroaryl, —(CH₂)ₚ—S(O)ᵣR^{k}, —(CH₂)ₚ—S(O)N(R^{L})₂, and —(CH₂)ₚ—N(R^{L})₂, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, —(CH₂)ₚ—S(O)ᵣR^{k}, —(CH₂)ₚ—S(O)N(R^{L})₂, and —(CH₂)ₚ—N(R^{L})₂, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₃₋₆cycloalkyl, —C₂₋₆cycloheteroalkyl, aryl, heteroaryl, —C(O)C₁₋₆alkyl, —(CH₂)ₚ—S(O)ᵣR^{k}, —(CH₂)ₚ—S(O)N(R^{L})₂, and —(CH₂)ₚ—N(R^{L})₂, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: oxo, —OH, halogen, —C₁₋₆alkyl, —C₃₋₆cycloalkyl, aryl, —C(O)C₁₋₆alkyl and —(CH₂)ₚ—N(R^{L})₂, wherein each R^{a} is unsubstituted or substituted with one to six substituents selected from halogen, CF₃, OH, C₁₋₆alkyl, and —OC₁₋₆alkyl. In a class of this embodiment, each R^{a} is independently selected from the group: oxo, —OH, F, —CH₃, —CH₂CH₃, —CF₂H, —CF₃, —CH₂OH, —C(CH₃)₂OH, cyclopropyl, phenyl, —C(O)CH₃ and —CH₂N(CH₃)₂, wherein each R^{a} is unsubstituted or substituted with one to six substituents selected from halogen, CF₃, OH, C₁₋₆ alkyl, and —OC₁₋₆alkyl.

In another embodiment of the present invention, each R^{a} is independently selected from the group: oxo, —OH, halogen, —C₁₋₆alkyl, —C₃₋₆cycloalkyl, aryl, —C(O)C₁₋₆alkyl, and —(CH₂)ₚ—N(R^{L})₂, wherein each R^{a} is unsubstituted or substituted with one to six substituents selected from halogen, CF₃, OH, C₁₋₆alkyl, and —OC₁₋₆alkyl. In a class of this embodiment of the present invention, each R^{a} is independently selected from the group: oxo, —OH, F, —CH₃, —CH₂CH₃, —CF₂H, —CF₃, —CH₂OH, —C(CH₃)₂OH, cyclopropyl, phenyl, —C(O)CH₃, and —CH₂N(CH₃)₂, wherein each R^{a} is unsubstituted or substituted with one to six substituents selected from F, CF₃, OH, C₁₋₆alkyl, and —OC₁₋₆alkyl.

In another embodiment of the present invention, each R^{a} is independently selected from the group: —OH and —C₁₋₆alkyl, wherein each R^{a} is unsubstituted or substituted with one to six substituents selected from halogen, CF₃, OH, C₁₋₆alkyl, and —OC₁₋₆alkyl. In a class of this embodiment of the present invention, each R^{a} is independently selected from the group: —OH and —C₁₋₆alkyl, wherein each R^{a} is unsubstituted or substituted with one to six substituents selected from F, CF₃, OH, C₁₋₆alkyl, and —OC₁₋₆alkyl. In another class of this embodiment of the present invention, each R^{a} is independently selected from the group: —OH, —CH₃, —CH₂CH₃, —CF₂H, —CF₃, —CH₂OH, and —C(CH₃)₂OH. In another class of this embodiment of the present invention, each R^{a} is independently selected from the group: —OH and —CH₃.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynylC₂₋₆cycloheteroalkyl, —C₂₋₆alkynyl-aryl, —C₂₋₆alkynyl-heteroaryl, —(CH₂)ₚ—O—C₁₋₆alkyl, —(CH₂)ₚ—O—C₂₋₆alkenyl, —(CH₂)ₚ—O—C₂₋₆alkynyl, —(CH₂)ₚ—O—C₃₋₆cycloalkyl, —(CH₂)ₚ—O—C₂₋₆cycloheteroalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆cycloheteroalkyl, —OC₁₋₆alkyl-aryl, —OC₁₋₆alkyl-heteroaryl, —(CH₂)ₚ—S(O)ᵣR^{k}, and —(CH₂)ₚ—N(R^{L})₂, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynylC₂₋₆cycloheteroalkyl, —C₂₋₆alkynyl-aryl, —C₂₋₆alkynyl-heteroaryl, —(CH₂)ₚ—O—C₁₋₆alkyl, —(CH₂)ₚ—O—C₂₋₆alkenyl, —(CH₂)ₚ—O—C₂₋₆alkynyl, —(CH₂)ₚ—O—C₃₋₆cycloalkyl, —(CH₂)ₚ—O—C₂₋₆cycloheteroalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆cycloheteroalkyl, —OC₁₋₆alkyl-aryl, —OC₁₋₆alkyl-heteroaryl, —(CH₂)ₚ—S(O)ᵣR^{k}, and —(CH₂)ₚ—N(R^{L})₂, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, and —(CH₂)ₚ—N(R^{L})₂, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₃₋₆cycloalkyl, —C₂₋₆cycloheteroalkyl, aryl, heteroaryl, —C(O)C₁₋₆alkyl, and —(CH₂)ₚ—N(R^{L})₂, wherein each R^{a} is unsubstituted or substituted with one to six substituents selected from halogen, CF₃, OH, C₁₋₆ alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynylC₂₋₆cycloheteroalkyl, —C₂₋₆alkynyl-aryl, —C₂₋₆alkynyl-heteroaryl, —(CH₂)ₚ—O—C₁₋₆alkyl, —(CH₂)ₚ—O—C₂₋₆alkenyl, —(CH₂)ₚ—O—C₂₋₆alkynyl, —(CH₂)ₚ—O—C₃₋₆cycloalkyl, —(CH₂)ₚ—O—C₂₋₆cycloheteroalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆cycloheteroalkyl, —OC₁₋₆alkyl-aryl, —OC₁₋₆alkyl-heteroaryl, and —(CH₂)ₚ—S(O)ᵣR^{k}, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynylC₂₋₆cycloheteroalkyl, —C₂₋₆alkynyl-aryl, —C₂₋₆alkynyl-heteroaryl, —(CH₂)ₚ—O—C₁₋₆alkyl, —(CH₂)ₚ—O—C₂₋₆alkenyl, —(CH₂)ₚ—O—C₂₋₆alkynyl, —(CH₂)ₚ—O—C₃₋₆cycloalkyl, —(CH₂)ₚ—O—C₂₋₆cycloheteroalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆cycloheteroalkyl, —OC₁₋₆alkyl-aryl, and —OC₁₋₆alkyl-heteroaryl, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynylC₂₋₆cycloheteroalkyl, —C₂₋₆alkynyl-aryl, —C₂₋₆alkynyl-heteroaryl, —(CH₂)ₚ—O—C₁₋₆alkyl, —(CH₂)ₚ—O—C₂₋₆alkenyl, —(CH₂)ₚ—O—C₂₋₆alkynyl, —(CH₂)ₚ—O—C₃₋₆cycloalkyl, —(CH₂)ₚ—O—C₂₋₆cycloheteroalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆cycloheteroalkyl, —OC₁₋₆alkyl-aryl, —OC₁₋₆alkyl-heteroaryl, and —(CH₂)ₚ—S(O)ᵣR^{k}, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, —C(O)C₁₋₆alkyl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —C₂₋₆alkenyl-C₃₋₆cycloalkyl, —C₂₋₆alkenyl-C₂₋₆cycloheteroalkyl, —C₂₋₆alkenyl-aryl, —C₂₋₆alkenyl-heteroaryl, —C₂₋₆alkynyl-C₃₋₆cycloalkyl, —C₂₋₆alkynylC₂₋₆cycloheteroalkyl, —C₂₋₆alkynyl-aryl, —C₂₋₆alkynyl-heteroaryl, —(CH₂)ₚ—O—C₁₋₆alkyl, —(CH₂)ₚ—O—C₂₋₆alkenyl, —(CH₂)ₚ—O—C₂₋₆alkynyl, —(CH₂)ₚ—O—C₃₋₆cycloalkyl, —(CH₂)ₚ—O—C₂₋₆cycloheteroalkyl, —(CH₂)ₚ—O-aryl, —(CH₂)ₚ—O-heteroaryl, —OC₁₋₆alkyl-C₃₋₆cycloalkyl, —OC₁₋₆alkyl-C₂₋₆cycloheteroalkyl, —OC₁₋₆alkyl-aryl, and —OC₁₋₆alkyl-heteroaryl, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₂₋₆alkenyl, —C₂₋₆alkynyl, —C₃₋₆cycloalkyl, —C₂₋₆cycloheteroalkyl, aryl, heteroaryl, and —C(O)C₁₋₆alkyl, wherein each R^{a} is unsubstituted or substituted with one to six substituents selected from halogen, CF₃, OH, C₁₋₆alkyl, and —OC₁₋₆alkyl.

In another embodiment of the present invention, each R^{a} is independently selected from the group: CN, oxo, —OH, halogen, —C₁₋₆alkyl, —O—C₁₋₆alkyl, —C₃₋₆cycloalkyl, —C₂₋₆cycloheteroalkyl, aryl, heteroaryl, and —C(O)C₁₋₆alkyl, wherein each R^{a} is unsubstituted or substituted with one to six substituents selected from halogen, CF₃, OH, C₁₋₆alkyl, and —OC₁₋₆alkyl.

In another embodiment of the present invention, each R^{b} is independently selected from the group: CN, —OH, oxo, halogen, —C₁₋₆alkyl, —O—C₁₋₆alkyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —(CH₂)_{q}—O—C₁₋₆alkyl, —(CH₂)_{q}—O—C₃₋₆cycloalkyl, —(CH₂)_{q}—O—C₂₋₆cycloheteroalkyl, —(CH₂)_{q}—O-aryl, —(CH₂)_{q}—O-heteroaryl, —OC₁₋₆alkyl-C₃₋₆cycloalkyl, —OC₁₋₆alkyl-C₂₋₆cycloheteroalkyl, —OC₁₋₆alkyl-aryl, —OC₁₋₆alkyl-heteroaryl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, —OH, oxo, —C₁₋₆alkyl, —O—C₁₋₆alkyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —(CH₂)_{q}—O—C₁₋₆alkyl, —(CH₂)_{q}—O—C₃₋₆cycloalkyl, —(CH₂)_{q}—O—C₂₋₆cycloheteroalkyl, —(CH₂)_{q}—O-aryl, —(CH₂)_{q}—O-heteroaryl, —OC₁₋₆alkyl-C₃₋₆cycloalkyl, —OC₁₋₆alkyl-C₂₋₆cycloheteroalkyl, —OC₁₋₆alkyl-aryl, —OC₁₋₆alkyl-heteroaryl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, oxo, —C₁₋₆alkyl, —O—C₁₋₆alkyl, —C₃₋₆cycloalkyl, —C₂₋₆cycloheteroalkyl, aryl, heteroaryl, —C₁₋₆alkyl-aryl, —C₁₋₆alkyl-heteroaryl, —C₁₋₆alkyl-C₃₋₆cycloalkyl, —C₁₋₆alkyl-C₂₋₆cycloheteroalkyl, —(CH₂)_{q}—O—C₁₋₆alkyl, —(CH₂)_{q}—O—C₃₋₆cycloalkyl, —(CH₂)_{q}—O—C₂₋₆cycloheteroalkyl, —(CH₂)_{q}—O-aryl, —(CH₂)_{q}—O-heteroaryl, —OC₁₋₆alkyl-C₃₋₆cycloalkyl, —OC₁₋₆alkyl-C₂₋₆cycloheteroalkyl, —OC₁₋₆alkyl-aryl, —OC₁₋₆alkyl-heteroaryl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, —OH, halogen, —C₁₋₆alkyl, —O—C₁₋₆alkyl, —C₃₋₆cycloalkyl, —C₂₋₆cycloheteroalkyl, aryl, heteroaryl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, halogen, —C₁₋₆alkyl, —O—C₁₋₆alkyl, —C₃₋₆cycloalkyl, —C₂₋₆cycloheteroalkyl, aryl, heteroaryl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, —OH, halogen, —C₁₋₆alkyl, —O—C₁₋₆alkyl, —C₃₋₆cycloalkyl, —C₂₋₆cycloheteroalkyl, aryl, heteroaryl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, halogen, —C₁₋₆alkyl, —O—C₁₋₆alkyl, —C₃₋₆cycloalkyl, —C₂₋₆cycloheteroalkyl, aryl, heteroaryl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, —OH, —C₁₋₆alkyl, —O—C₁₋₆alkyl, —C₃₋₆cycloalkyl, —C₂₋₆cycloheteroalkyl, aryl, heteroaryl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, —C₁₋₆alkyl, —O—C₁₋₆alkyl, —C₃₋₆cycloalkyl, —C₂₋₆cycloheteroalkyl, aryl, heteroaryl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, halogen, —C₁₋₆alkyl, —O—C₁₋₆alkyl, —C₃₋₆cycloalkyl, —C₂₋₆cycloheteroalkyl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In a class of this embodiment, each R^{b} is independently selected from the group: OH, Cl, F, —CH₃, —CF₃, —CF₂H, —OCF₂H, and cyclopropane, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, halogen, —C₁₋₆alkyl, —O—C₁₋₆alkyl, —C₃₋₆cycloalkyl, —C₂₋₆cycloheteroalkyl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In a class of this embodiment, each R^{b} is independently selected from the group: Cl, F, —CH₃, —CF₃, —CF₂H, —OCF₂H, and cyclopropane, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, —OH, —C₁₋₆alkyl, —O—C₁₋₆alkyl, —C₃₋₆cycloalkyl, —C₂₋₆cycloheteroalkyl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In a class of this embodiment, each R^{b} is independently selected from the group: OH, —CH₃, —CF₃, —CF₂H, —OCF₂H, and cyclopropane, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, —C₁₋₆alkyl, —O—C₁₋₆alkyl, —C₃₋₆cycloalkyl, —C₂₋₆cycloheteroalkyl, —(CH₂)_{q}—S(O)ᵣR^{m}, —(CH₂)_{q}N(Rⁿ)₂, —C(O)R^{o}, and —C(O)NRⁿ, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In a class of this embodiment, each R^{b} is independently selected from the group: —CH₃, —CF₃, —CF₂H, —OCF₂H, and cyclopropane, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, —OH, —CH₃, —CF₃, —CF₂H, —OCF₂H, and cyclopropane, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, —CH₃, —CF₃, —CF₂H, —OCF₂H, and cyclopropane, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, —OH, halogen, —C₁₋₆alkyl, —O—C₁₋₆alkyl, —C₃₋₆cycloalkyl, and —C₂₋₆cycloheteroalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, halogen, —C₁₋₆alkyl, —O—C₁₋₆alkyl, —C₃₋₆cycloalkyl, and —C₂₋₆cycloheteroalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, —OH, —C₁₋₆alkyl, —O—C₁₋₆alkyl, —C₃₋₆cycloalkyl, and —C₂₋₆cycloheteroalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, —C₁₋₆alkyl, —O—C₁₋₆alkyl, —C₃₋₆cycloalkyl, and —C₂₋₆cycloheteroalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is selected from the group: CN, —OH, oxo, halogen, —C₁₋₆alkyl, —O—C₁₋₆alkyl, and —C₃₋₆cycloalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is selected from the group: CN, oxo, halogen, —C₁₋₆alkyl, —O—C₁₋₆alkyl, and —C₃₋₆cycloalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: CN, —OH, oxo, —C₁₋₆alkyl, —O—C₁₋₆alkyl, and —C₃₋₆cycloalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: CN, oxo, —C₁₋₆alkyl, —O—C₁₋₆alkyl, and —C₃₋₆cycloalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is selected from the group: —OH, halogen, —C₁₋₆alkyl, —O—C₁₋₆alkyl, and —C₃₋₆cycloalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is selected from the group: halogen, —C₁₋₆alkyl, —O—C₁₋₆alkyl, and —C₃₋₆cycloalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: —OH, —C₁₋₆alkyl, —O—C₁₋₆alkyl, and —C₃₋₆cycloalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In another embodiment of the present invention, each R^{b} is independently selected from the group: —C₁₋₆alkyl, —O—C₁₋₆alkyl, and —C₃₋₆cycloalkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl.

In another embodiment of the present invention, each R^{b} is independently selected from the group: —OH, —C₁₋₆alkyl, and —O—C₁₋₆alkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In a class of this embodiment, each R^{b} is independently selected from the group: —OH, —C₁₋₆alkyl, and —O—C₁₋₆alkyl, wherein each R^{b} is unsubstituted or substituted with one to six substituents selected from F, CF₃, CF₂H, OCF₃, CN, CH₂CF₃, CF₂CH₃, —C₁₋₆alkyl, and —OC₁₋₆alkyl. In another class of this embodiment, each R^{b} is independently OH, —CH₃, —CF₃, —CF₂H, or —OCF₂H. In another embodiment of the present invention, each R^{b} is independently selected from the group: —C₁₋₆alkyl, and —O—C₁₋₆alkyl, wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl. In a class of this embodiment, each R^{b} is independently selected from the group: —C₁₋₆alkyl, and —O—C₁₋₆alkyl, wherein each R^{b} is unsubstituted or substituted with one to six substituents selected from F, CF₃, CF₂H, OCF₃, CN, CH₂CF₃, CF₂CH₃, —C₁₋₆alkyl, and —OC₁₋₆alkyl. In another class of this embodiment, each R^{b} is independently —CH₃, —CF₃, —CF₂H, or —OCF₂H.

In another class of this embodiment, each R^{b} is independently selected from the group: —OH, —CH₃, —CF₃, and —OCF₂H. In another class of this embodiment, each R^{b} is independently selected from the group: —OH, —CF₃, and —OCF₂H. In another embodiment, each R^{b} is independently selected from the group: —OH, —CH₃, —CF₃, —OCF₂H, and cyclopropane. In another class of this embodiment, each R^{b} is independently selected from the group: —CH₃, —CF₃, and —OCF₂H. In another class of this embodiment, each R^{b} is independently selected from the group: —CF₃, and —OCF₂H. In another embodiment, each R^{b} is independently selected from the group: —OH, —CH₃, —CF₃, —OCF₂H, and cyclopropane.

In another embodiment, each R^{b} is independently selected from the group: —OH, and —CF₃. In another embodiment, each R^{b} is CF₃.

In another embodiment of the present invention, each R^{c} is independently selected from the group: hydrogen, halogen, and —C₁₋₆alkyl, wherein alkyl is unsubstituted or substituted with one to three substituents selected from: CF₃, halogen, OH and —OC₁₋₆alkyl.

In another embodiment, each R^{c} is independently selected from the group: hydrogen, and —C₁₋₆alkyl, wherein alkyl is unsubstituted or substituted with one to three substituents selected from: CF₃, halogen, OH and —OC₁₋₆alkyl. In a class of this embodiment, each R^{c} is —CH₃. In another embodiment, R^{c} is hydrogen.

In another embodiment, R^{c} is —C₁₋₆alkyl, wherein alkyl is unsubstituted or substituted with one to three substituents selected from: CF₃, halogen, OH and —OC₁₋₆alkyl. In a class of this embodiment, each R^{c} is —CH₃.

In another embodiment of the present invention, each R^{d} is independently selected from the group: CF₃, halogen, and —C₁₋₆alkyl, wherein alkyl is unsubstituted or substituted with one to three substituents selected from: CF₃, halogen, OH and —OC₁₋₆alkyl.

In another embodiment of the present invention, each R^{d} is independently selected from the group: halogen and —C₁₋₆alkyl, wherein alkyl is unsubstituted or substituted with one to three substituents selected from: CF₃, halogen, OH and —OC₁₋₆alkyl. In a class of this embodiment, each R^{d} is independently selected from the group: halogen and —CH₃. In another embodiment of the present invention, each R^{d} is halogen.

In another embodiment of the present invention, each R^{d} is —C₁₋₆alkyl, wherein alkyl is unsubstituted or substituted with one to three substituents selected from: CF₃, halogen, OH and —OC₁₋₆alkyl. In a class of this embodiment, each R^{d} is —CH₃.

In another embodiment of the present invention, each R^{e} is independently selected from the group: CF₃, halogen, and —C₁₋₆alkyl, wherein alkyl is unsubstituted or substituted with one to three substituents selected from: CF₃, halogen, OH and —OC₁₋₆alkyl.

In another embodiment of the present invention, each R^{e} is independently selected from the group: halogen and —C₁₋₆alkyl, wherein alkyl is unsubstituted or substituted with one to three substituents selected from: CF₃, halogen, OH and —OC₁₋₆alkyl. In a class of this embodiment, each R^{e} is independently selected from the group: halogen and —CH₃. In another embodiment of the present invention, each R^{e} is halogen.

In another embodiment of the present invention, each R^{e} is —C₁₋₆alkyl, wherein alkyl is unsubstituted or substituted with one to three substituents selected from: CF₃, halogen, OH and —OC₁₋₆alkyl. In a class of this embodiment, each R^{e} is —CH₃.

In another embodiment of the present invention, each R^{f} is independently selected from the group: CF₃, halogen, and —C₁₋₆alkyl, wherein alkyl is unsubstituted or substituted with one to three substituents selected from: CF₃, halogen, OH and —OC₁₋₆alkyl.

In another embodiment of the present invention, each R^{f} is independently selected from the group: halogen and —C₁₋₆alkyl, wherein alkyl is unsubstituted or substituted with one to three substituents selected from: CF₃, halogen, OH and —OC₁₋₆alkyl. In a class of this embodiment, each R^{f} is independently selected from the group: halogen and —CH₃. In another embodiment of the present invention, each R^{f} is halogen.

In another embodiment of the present invention, each R^{f} is —C₁₋₆alkyl, wherein alkyl is unsubstituted or substituted with one to three substituents selected from: CF₃, halogen, OH and —OC₁₋₆alkyl. In a class of this embodiment, each R^{f} is —CH₃.

In another embodiment of the present invention, each R^{g} is independently selected from the group: CF₃, halogen, and —C₁₋₆alkyl, wherein alkyl is unsubstituted or substituted with one to three substituents selected from: CF₃, halogen, OH and —OC₁₋₆alkyl.

In another embodiment of the present invention, each R^{g} is independently selected from the group: halogen and —C₁₋₆alkyl, wherein alkyl is unsubstituted or substituted with one to three substituents selected from: CF₃, halogen, OH and —OC₁₋₆alkyl. In a class of this embodiment, each R^{g} is independently selected from the group: halogen and —CH₃. In another embodiment of the present invention, each R^{g} is halogen.

In another embodiment of the present invention, each R^{g} is —C₁₋₆alkyl, wherein alkyl is unsubstituted or substituted with one to three substituents selected from: CF₃, halogen, OH and —OC₁₋₆alkyl. In a class of this embodiment, each R^{g} is —CH₃.

In another embodiment of the present invention, each R^{h} is independently selected from the group: hydrogen, —C₁₋₆alkyl, —C₃₋₆cycloalkyl, and —C₂₋₆cycloheteroalkyl, wherein alkyl, cycloalkyl and cycloheteroalkyl are unsubstituted or substituted with one to three substituents selected from: —CF₃, halogen, OH and —OC₁₋₆alkyl. In a class of this embodiment, R^{h} is —C₂₋₆cycloheteroalkyl, wherein cycloheteroalkyl is unsubstituted or substituted with one to three substituents selected from: —CF₃, halogen, OH and —OC₁₋₆alkyl.

In another embodiment of the present invention, each R^{h} is independently selected from the group: hydrogen, —C₁₋₆alkyl, and —C₃₋₆cycloalkyl, wherein alkyl and cycloalkyl are unsubstituted or substituted with one to three substituents selected from: —CF₃, halogen, OH and —OC₁₋₆alkyl. In a class of this embodiment, R^{h} is —C₃₋₆cycloalkyl, wherein cycloalkyl is unsubstituted or substituted with one to three substituents selected from: —CF₃, halogen, OH and —OC₁₋₆alkyl.

In another embodiment of the present invention, each R^{h} is independently selected from the group: hydrogen and —C₁₋₆alkyl, wherein alkyl is unsubstituted or substituted with one to three substituents selected from: —CF₃, halogen, OH and —OC₁₋₆alkyl. In another embodiment of the present invention, R^{h} is hydrogen. In another embodiment of the present invention, R^{h} is —C₁₋₆alkyl, wherein alkyl is unsubstituted or substituted with one to three substituents selected from: —CF₃, halogen, OH and —OC₁₋₆alkyl.

In another embodiment of the present invention, each Rⁱ is independently selected from the group: hydrogen, —C₁₋₆alkyl, —C₃₋₆cycloalkyl, and —C₂₋₆cycloheteroalkyl, wherein alkyl, cycloalkyl and cycloheteroalkyl are unsubstituted or substituted with one to three substituents selected from: CF₃, halogen, OH and —OC₁₋₆alkyl. In a class of this embodiment, Rⁱ is —C₂₋₆cycloheteroalkyl, wherein cycloheteroalkyl is unsubstituted or substituted with one to three substituents selected from: —CF₃, halogen, OH and —OC₁₋₆alkyl.

In another embodiment of the present invention, each Rⁱ is independently selected from the group: hydrogen, —C₁₋₆alkyl, and —C₃₋₆cycloalkyl, wherein alkyl and cycloalkyl are unsubstituted or substituted with one to three substituents selected from: —CF₃, halogen, OH and —OC₁₋₆alkyl. In a class of this embodiment, Rⁱ is —C₃₋₆cycloalkyl, wherein cycloalkyl is unsubstituted or substituted with one to three substituents selected from: —CF₃, halogen, OH and —OC₁₋₆alkyl.

In another embodiment of the present invention, each Rⁱ is independently selected from the group: hydrogen and —C₁₋₆alkyl, wherein alkyl is unsubstituted or substituted with one to three substituents selected from: —CF₃, halogen, OH and —OC₁₋₆alkyl. In another embodiment of the present invention, Rⁱ is hydrogen. In another embodiment of the present invention, Rⁱ is —C₁₋₆alkyl, wherein alkyl is unsubstituted or substituted with one to three substituents selected from: —CF₃, halogen, OH and —OC₁₋₆alkyl.

In another embodiment of the present invention, each R^{j} is independently selected from the group: hydrogen, —C₁₋₆alkyl, —C₃₋₆cycloalkyl, and —C₂₋₆cycloheteroalkyl, wherein alkyl, cycloalkyl and cycloheteroalkyl are unsubstituted or substituted with one to three substituents selected from: CF₃, halogen, OH and —OC₁₋₆alkyl. In a class of this embodiment, R^{j} is —C₂₋₆cycloheteroalkyl, wherein cycloheteroalkyl is unsubstituted or substituted with one to three substituents selected from: —CF₃, halogen, OH and —OC₁₋₆alkyl.

In another embodiment of the present invention, each R^{j} is independently selected from the group: hydrogen, —C₁₋₆alkyl, and —C₃₋₆cycloalkyl, wherein alkyl and cycloalkyl are unsubstituted or substituted with one to three substituents selected from: —CF₃, halogen, OH and —OC₁₋₆alkyl. In a class of this embodiment, R^{j} is —C₃₋₆cycloalkyl, wherein cycloalkyl is unsubstituted or substituted with one to three substituents selected from: —CF₃, halogen, OH and —OC₁₋₆alkyl.

In another embodiment of the present invention, each R^{j} is independently selected from the group: hydrogen and —C₁₋₆alkyl, wherein alkyl is unsubstituted or substituted with one to three substituents selected from: —CF₃, halogen, OH and —OC₁₋₆alkyl. In another embodiment of the present invention, R^{j} is hydrogen. In another embodiment of the present invention, R^{j} is —C₁₋₆alkyl, wherein alkyl is unsubstituted or substituted with one to three substituents selected from: —CF₃, halogen, OH and —OC₁₋₆alkyl.

In another embodiment of the present invention, each R^{k} is independently selected from the group: —C₁₋₆alkyl, —C₃₋₆cycloalkyl, and —C₂₋₆cycloheteroalkyl, wherein alkyl, cycloalkyl, and cycloheteroalkyl are unsubstituted or substituted with one to three substituents selected from: —CF₃, halogen, OH and —OC₁₋₆alkyl. In a class of this embodiment, R^{k} is —C₂₋₆cycloheteroalkyl, wherein cycloheteroalkyl is unsubstituted or substituted with one to three substituents selected from: —CF₃, halogen, OH and —OC₁₋₆alkyl.

In another embodiment of the present invention, each R^{k} is independently selected from the group: —C₁₋₆alkyl and —C₃₋₆cycloalkyl, wherein alkyl and cycloalkyl are unsubstituted or substituted with one to three substituents selected from: —CF₃, halogen, OH and —OC₁₋₆alkyl. In a class of this embodiment, R^{k} is —C₃₋₆cycloalkyl, wherein cycloalkyl is unsubstituted or substituted with one to three substituents selected from: —CF₃, halogen, OH and —OC₁₋₆alkyl.

In another embodiment of the present invention, R^{k} is —C₁₋₆alkyl, wherein alkyl is unsubstituted or substituted with one to three substituents selected from: —CF₃, halogen, OH and —OC₁₋₆alkyl.

In another embodiment of the present invention, each R^{L} is independently selected from the group: hydrogen, —C₁₋₆alkyl, —C₃₋₆cycloalkyl, and —C₂₋₆cycloheteroalkyl, wherein alkyl, cycloalkyl, and cycloheteroalkyl are unsubstituted or substituted with one to three substituents selected from: —CF₃, halogen, OH and —OC₁₋₆alkyl. In a class of this embodiment, R^{L} is —C₂₋₆cycloheteroalkyl, wherein cycloheteroalkyl is unsubstituted or substituted with one to three substituents selected from: —CF₃, halogen, OH and —OC₁₋₆alkyl.

In another embodiment of the present invention, each R^{L} is independently selected from the group: hydrogen, —C₁₋₆alkyl, and —C₃₋₆cycloalkyl, wherein alkyl and cycloalkyl are unsubstituted or substituted with one to three substituents selected from: —CF₃, halogen, OH and —OC₁₋₆alkyl. In a class of this embodiment, R^{L} is —C₃₋₆cycloalkyl, wherein cycloalkyl is unsubstituted or substituted with one to three substituents selected from: —CF₃, halogen, OH and —OC₁₋₆alkyl.

In another embodiment of the present invention, each R^{L} is independently selected from the group: hydrogen and —C₁₋₆alkyl, wherein alkyl is unsubstituted or substituted with one to three substituents selected from: —CF₃, halogen, OH and —OC₁₋₆alkyl. In another embodiment of the present invention, R^{L} is hydrogen. In another embodiment of the present invention, R^{L} is —C₁₋₆alkyl, wherein alkyl is unsubstituted or substituted with one to three substituents selected from: —CF₃, halogen, OH and —OC₁₋₆alkyl. In a class of this embodiment, R^{L} is —CH₃.

In another embodiment of the present invention, each R^{m} is independently selected from the group: —C₁₋₆alkyl, —C₃₋₆cycloalkyl, and —C₂₋₆cycloheteroalkyl, wherein alkyl, cycloalkyl and cycloheteroalkyl are unsubstituted or substituted with one to three substituents selected from: —CF₃, halogen, OH and —OC₁₋₆alkyl. In a class of this embodiment, R^{m} is —C₂₋₆cycloheteroalkyl, wherein cycloheteroalkyl is unsubstituted or substituted with one to three substituents selected from: —CF₃, halogen, OH and —OC₁₋₆alkyl.

In another embodiment of the present invention, each R^{m} is independently selected from the group: —C₁₋₆alkyl and —C₃₋₆cycloalkyl, wherein alkyl and cycloalkyl are unsubstituted or substituted with one to three substituents selected from: —CF₃, halogen, OH and —OC₁₋₆alkyl. In a class of this embodiment, R^{m} is —C₃₋₆cycloalkyl, wherein cycloalkyl is unsubstituted or substituted with one to three substituents selected from: —CF₃, halogen, OH and —OC₁₋₆alkyl.

In another embodiment of the present invention, R^{m} is —C₁₋₆alkyl, wherein alkyl, wherein alkyl is unsubstituted or substituted with one to three substituents selected from: —CF₃, halogen, OH and —OC₁₋₆alkyl.

In another embodiment of the present invention, each Rⁿ is independently selected from the group: hydrogen, —C₁₋₆alkyl, —C₃₋₆cycloalkyl, and —C₂₋₆cycloheteroalkyl, wherein alkyl, cycloalkyl, and cycloheteroalkyl is unsubstituted or substituted with one to three substituents selected from: —CF₃, halogen, OH and —OC₁₋₆alkyl. In a class of this embodiment, Rⁿ is —C₂₋₆cycloheteroalkyl, wherein cycloheteroalkyl is unsubstituted or substituted with one to three substituents selected from: —CF₃, halogen, OH and —OC₁₋₆alkyl.

In another embodiment of the present invention, each Rⁿ is independently selected from the group: hydrogen, —C₁₋₆alkyl, and —C₃₋₆cycloalkyl, wherein alkyl and cycloalkyl are unsubstituted or substituted with one to three substituents selected from: —CF₃, halogen, OH and —OC₁₋₆alkyl. In a class of this embodiment, Rⁿ is —C₃₋₆cycloalkyl, wherein cycloalkyl is unsubstituted or substituted with one to three substituents selected from: —CF₃, halogen, OH and —OC₁₋₆alkyl.

In another embodiment of the present invention, each Rⁿ is independently selected from the group: hydrogen and C₁₋₆alkyl, wherein alkyl is unsubstituted or substituted with one to three substituents selected from: —CF₃, halogen, OH and —OC₁₋₆alkyl. In another embodiment of the present invention, each Rⁿ is hydrogen. In another embodiment of the present invention, each Rⁿ is —C₁₋₆alkyl, wherein alkyl is unsubstituted or substituted with one to three substituents selected from: —CF₃, halogen, OH and —OC₁₋₆alkyl.

In another embodiment of the present invention, each R^{o} is independently selected from the group: OH, —C₁₋₆alkyl, —C₃₋₆cycloalkyl, and —C₂₋₆cycloheteroalkyl, wherein alkyl, cycloalkyl and cycloheteroalkyl is unsubstituted or substituted with one to three substituents selected from —CF₃, halogen, OH and —OC₁₋₆alkyl.

In another embodiment of the present invention, each R^{o} is independently selected from the group: OH and —C₁₋₆alkyl, wherein alkyl is unsubstituted or substituted with one to three substituents selected from —CF₃, halogen, OH and —OC₁₋₆alkyl. In another embodiment of the present invention, each R^{o} OH. In another embodiment of the present invention, each R^{o} is —C₁₋₆alkyl, wherein alkyl is unsubstituted or substituted with one to three substituents selected from —CF₃, halogen, OH and —OC₁₋₆alkyl.

In another embodiment of the present invention, r is 0, 1, 2, 3, 4, 5 or 6. In another embodiment, r is 0, 1, 2, 3, 4, or 5. In another embodiment, r is 1, 2, 3, 4, 5 or 6. In another embodiment, r is 1, 2, 3, 4 or 5. In another embodiment, r is 0, 1, 2, 3, or 4. In another embodiment, r is 1, 2, 3, or 4. In another embodiment, r is 0, 1, 2, or 3. In another embodiment, r is 1, 2, or 3. In another embodiment, r is 0, 1 or 2. In another embodiment, r is 1 or 2. In another embodiment, r is 0. In another embodiment, r is 1. In another embodiment, r is 2. In another embodiment, r is 3. In another embodiment, r is 4. In another embodiment, r is 5. In another embodiment, r is 6.

In another embodiment of the present invention, s is 0, 1, 2, 3, 4, 5 or 6. In another embodiment, s is 0, 1, 2, 3, 4, or 5. In another embodiment, s is 1, 2, 3, 4, 5 or 6. In another embodiment, s is 1, 2, 3, 4 or 5. In another embodiment, s is 0, 1, 2, 3, or 4. In another embodiment, s is 1, 2, 3, or 4. In another embodiment, s is 0, 1, 2, or 3. In another embodiment, s is 1, 2, or 3. In another embodiment, s is 0, 1 or 2. In another embodiment, s is 1 or 2. In another embodiment, s is 0. In another embodiment, s is 1. In another embodiment, s is 2. In another embodiment, s is 3. In another embodiment, s is 4. In another embodiment, s is 5. In another embodiment, s is 6.

In another embodiment of the present invention, t is 0, 1, 2, 3, 4, 5 or 6. In another embodiment, t is 0, 1, 2, 3, 4, or 5. In another embodiment, t is 1, 2, 3, 4, 5 or 6. In another embodiment, t is 1, 2, 3, 4 or 5. In another embodiment, t is 0, 1, 2, 3, or 4. In another embodiment, t is 1, 2, 3, or 4. In another embodiment, t is 0, 1, 2, or 3. In another embodiment, t is 1, 2, or 3. In another embodiment, t is 0, 1 or 2. In another embodiment, t is 1 or 2. In another embodiment, t is 0. In another embodiment, t is 1. In another embodiment, t is 2. In another embodiment, t is 3. In another embodiment, t is 4. In another embodiment, t is 5. In another embodiment, t is 6.

In another embodiment of the present invention, u is 0, 1, 2, 3, 4, 5 or 6. In another embodiment, u is 0, 1, 2, 3, 4, or 5. In another embodiment, u is 1, 2, 3, 4, 5 or 6. In another embodiment, u is 1, 2, 3, 4 or 5. In another embodiment, u is 0, 1, 2, 3, or 4. In another embodiment, u is 1, 2, 3, or 4. In another embodiment, u is 0, 1, 2, or 3. In another embodiment, u is 1, 2, or 3. In another embodiment, u is 0, 1 or 2. In another embodiment, u is 1 or 2. In another embodiment, u is 0. In another embodiment, u is 1. In another embodiment, u is 2. In another embodiment, u is 3. In another embodiment, u is 4. In another embodiment, u is 5. In another embodiment, u is 6.

In another embodiment of the present invention, p is 0, 1, 2, 3, 4, 5 or 6. In another embodiment, p is 0, 1, 2, 3, 4, or 5. In another embodiment, p is 1, 2, 3, 4, 5 or 6. In another embodiment, p is 1, 2, 3, 4 or 5. In another embodiment, p is 0, 1, 2, 3, or 4. In another embodiment, p is 1, 2, 3, or 4. In another embodiment, p is 0, 1, 2, or 3. In another embodiment, p is 1, 2, or 3. In another embodiment, p is 0, 1 or 2. In another embodiment, p is 1 or 2. In another embodiment, p is 0. In another embodiment, p is 1. In another embodiment, p is 2. In another embodiment, p is 3. In another embodiment, p is 4. In another embodiment, p is 5. In another embodiment, p is 6.

In another embodiment of the present invention, q is 0, 1, 2, 3, 4, 5 or 6. In another embodiment, q is 0, 1, 2, 3, 4, or 5. In another embodiment, q is 1, 2, 3, 4, 5 or 6. In another embodiment, q is 1, 2, 3, 4 or 5. In another embodiment, q is 0, 1, 2, 3, or 4. In another embodiment, q is 1, 2, 3, or 4. In another embodiment, q is 0, 1, 2, or 3. In another embodiment, q is 1, 2, or 3. In another embodiment, q is 0, 1 or 2. In another embodiment, q is 1 or 2. In another embodiment, q is 0. In another embodiment, q is 1. In another embodiment, q is 2. In another embodiment, q is 3. In another embodiment, q is 4. In another embodiment, q is 5. In another embodiment, q is 6.

In another embodiment of the present invention, the invention relates to compounds of structural formula Ia:

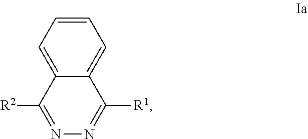

or a pharmaceutically acceptable salt thereof.

In another embodiment of the present invention, the invention relates to compounds of structural formula Ib:

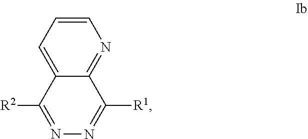

or a pharmaceutically acceptable salt thereof.

In another embodiment of the present invention, the invention relates to compounds of structural formula Ic:

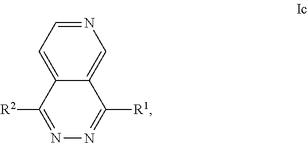

or a pharmaceutically acceptable salt thereof.

In another embodiment of the present invention, the invention relates to compounds of structural formula Id:

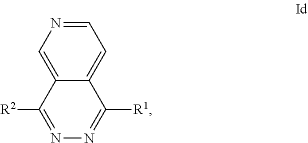

or a pharmaceutically acceptable salt thereof.

In another embodiment of the present invention, the invention relates to compounds of structural formula Ie:

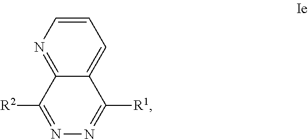

or a pharmaceutically acceptable salt thereof.

In another embodiment of the present invention, the invention relates to compounds of structural formula If:

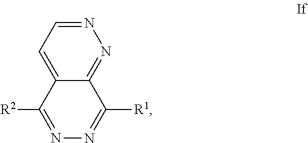

or a pharmaceutically acceptable salt thereof.

In another embodiment of the present invention, the invention relates to compounds of structural formula Ig:

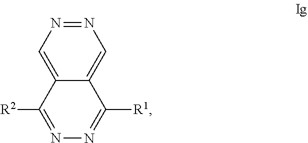

or a pharmaceutically acceptable salt thereof.

In another embodiment of the present invention, the invention relates to compounds of structural formula Ih:

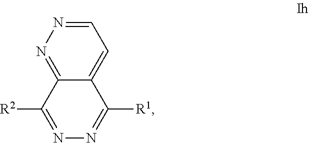

or a pharmaceutically acceptable salt thereof.

In another embodiment of the present invention, the invention relates to compounds of structural formula Ii:

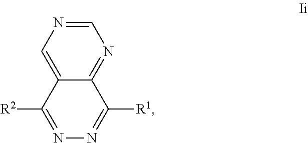

or a pharmaceutically acceptable salt thereof.

In another embodiment of the present invention, the invention relates to compounds of structural formula Ij:

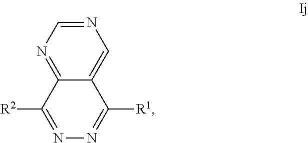

or a pharmaceutically acceptable salt thereof.

In another embodiment of the present invention, the invention relates to compounds of structural formula Ik:

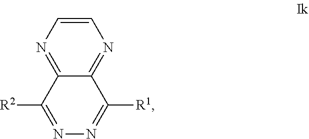

The compound of structural formula I, includes the compounds of structural formulas Ia, Ib, Ic, Id, Ie, If, Ig, Ih, Ii, Ij and Ik, and pharmaceutically acceptable salts, hydrates and solvates thereof.

Another embodiment of the present invention relates to compounds of structural formula I wherein:
R¹ is C₂₋₁₂cycloheteroalkyl, wherein R¹ is unsubstituted or substituted with one to six substituents selected from R^{a};
R² is heteroaryl, wherein R² is unsubstituted or substituted with one to five substituents selected from R^{b};
R³ is selected from the group:
   1) hydrogen, and
   2) —C₁₋₆alkyl,
wherein R³ is unsubstituted or substituted with one to five substituents selected from R^{d};
R⁴ is selected from the group:
   1) hydrogen,
   2) OH,
   3) CN,
   4) CF₃,
   5) CF₂H,
   6) —C₁₋₆alkyl,
   7) —O—C₁₋₆alkyl,
   8) halogen, and
   9) —C₁₋₆alkyl-O—C₁₋₆alkyl,
wherein R⁴ is unsubstituted or substituted with one to five substituents selected from R^{e};
R⁵ is selected from the group:
   1) hydrogen,
   2) OH,
   3) CN,
   4) CF₃,
   5) CF₂H,
   6) —C₁₋₆alkyl,
   7) —O—C₁₋₆alkyl,
   8) halogen, and
   9) —C₁₋₆alkyl-O—C₁₋₆alkyl,
wherein R⁵ is unsubstituted or substituted with one to five substituents selected from R^{f}; and
R⁶ is selected from the group:
   1) hydrogen,
   2) —C₁₋₆alkyl, and
   3) —O—C₁₋₆alkyl,
wherein R⁶ is unsubstituted or substituted with one to five substituents selected from R^{g};
and the other substituents are as defined above;
or a pharmaceutically acceptable salt thereof.

Another embodiment of the present invention relates to compounds of structural formula I wherein:
W is CR⁶;
R¹ is selected from the group:
   1) morpholine,
   2) thiomorpholine,
   3) piperidine,
   4) piperazine,
   5) pyrrolidine,
   6) tetrahydropyran,
   7) octahydro-1H-pyrrolo[2,3-c]pyridine,
   8) 3-azabicyclo[3.1.0]hexane,
   9) 5-azaspiro[2.4]heptane,
   10) 1-oxa-7-azaspiro[4.4]nonane,
   11) 1-oxa-8-azaspiro[4.5]decane,
   12) 3-oxa-1,8-diazaspiro[4.5]decane,
   13) 2,8-diazaspiro[4.5]decane,
   14) 1-oxa-3,8-diazaspiro[4.5]decane,
   15) 2-oxa-8-azaspiro[4.5]decane,
   16) 1,8-diazaspiro[4.5]decane, and
   17) 1-oxa-4,9-diazaspiro[5.5]undecane,
wherein R¹ is unsubstituted or substituted with one to six substituents selected from R^{a};
R² is aryl, wherein R² is unsubstituted or substituted with one to five substituents selected from R^{b};
R³ is hydrogen;
R⁴ is selected from the group:
   1) hydrogen, and
   2) —C₁₋₆alkyl,
wherein R⁴ is unsubstituted or substituted with one to five substituents selected from R^{e};
R⁵ is selected from the group:
   1) hydrogen,
   2) —C₁₋₆alkyl, and
   3) halogen,
wherein R⁵ is unsubstituted or substituted with one to five substituents selected from R^{f}; and R⁶ is hydrogen;
and the other substituents are as defined above;
or a pharmaceutically acceptable salt thereof.

Illustrative, but non-limiting, examples of compounds of the present invention that are useful as inhibitors of the NLRP3 are the following compounds:
1) (S)-1-(4-(2-hydroxy-4-(trifluoromethyl)phenyl)pyrido[3,4-d]pyridazin-1-yl)-3-methylpyrrolidin-3-ol;
2) (S)-1-(1-(2-hydroxy-4-(trifluoromethyl)phenyl)pyrido[3,4-d]-pyridazin-4-yl)-3-methylpyrrolidin-3-ol;
3) (cis)-4-(5-(2-(difluoromethoxy)-4-(trifluoromethyl)phenyl)pyrido[2,3-d]pyridazin-8-yl)-2,6-dimethylmorpholine;
4) 2-(4-((cis)-2,6-dimethylmorpholino)pyrido[3,4-d]pyridazin-1-yl)-5-(trifluoromethyl)phenol;
5) 2-(1-((cis)-2,6-dimethylmorpholino)pyrido[3,4-d]pyridazin-4-yl)-5-(trifluoromethyl)phenol;
6) 2-(5-((cis)-2,6-dimethylmorpholino)pyrido[2,3-d]pyridazin-8-yl)-5-(trifluoromethyl)phenol;
7) (cis)-4-(1-(2-(difluoromethoxy)-4-(trifluoromethyl)phenyl)pyrido[3,4-d]pyridazin-4-yl)-2,6-dimethylmorpholine;
8) 2-(8-((cis)-2,6-dimethylmorpholino)pyridazino[4,5-c]pyridazin-5-yl)-5-(trifluoromethyl)-phenol;
9) 2-(4-((cis)-2,6-dimethylmorpholino)pyridazino[4,5-d]pyridazin-1-yl)-5-(trifluoromethyl)-phenol;
10) (S)-1-(5-(2-hydroxy-4-(trifluoromethyl)phenyl)-2-methylpyrido[2,3-d]pyridazin-8-yl)-3-methylpyrrolidin-3-ol;
11) (cis)-4-(1-(benzofuran-5-yl)pyrido[3,4-d]pyridazin-4-yl)-2,6-dimethylmorpholine;
12) (S)-1-(5-(2-hydroxy-4-(trifluoromethyl)phenyl)-2-(trifluoromethyl)pyrido[2,3-d]pyridazin-8-yl)-3-methylpyrrolidin-3-ol;
13) (3S,4s,5R)-1-(4-(2-hydroxy-4-(trifluoromethyl)phenyl)pyridazino[4,5-d]pyridazin-1-yl)-3,4,5-trimethylpiperidin-4-ol;
14) 2-(4-((cis)-2,6-dimethylmorpholino)-5-methylphthalazin-1-yl)-5-(trifluoromethyl)phenol;
15) 2-(4-((cis)-2,6-dimethylmorpholino)-8-methylphthalazin-1-yl)-5-(trifluoromethyl)phenol;
16) (3S,4s,5R)-1-(1-(2-hydroxy-4-(trifluoromethyl)phenyl)pyrido[3,4-d]pyridazin-4-yl)-3,4,5-trimethylpiperidin-4-ol;
17) (3S,4s,5R)-1-(4-(2-hydroxy-4-(trifluoromethyl)phenyl)pyrido[3,4-d]pyridazin-1-yl)-3,4,5-trimethylpiperidin-4-ol;
18) (3S,4r,5R)-1-(1-(2-hydroxy-4-(trifluoromethyl)phenyl)pyrido[3,4-d]pyridazin-4-yl)-3,5-dimethylpiperidin-4-ol;
19) (3S,4r,5R)-1-(4-(2-hydroxy-4-(trifluoromethyl)phenyl)pyrido[3,4-d]pyridazin-1-yl)-3,5-dimethylpiperidin-4-ol;
20) 2-(4-((cis)-2,6-dimethylmorpholino)-6-methylphthalazin-1-yl)-5-(trifluoromethyl)phenol;
21) 2-(4-((cis)-2,6-dimethylmorpholino)-7-methylphthalazin-1-yl)-5-(trifluoromethyl)phenol;
22) (S)-1-(5-(2-hydroxy-4-(trifluoromethyl)phenyl)pyrido[2,3-d]-pyridazin-8-yl)-3-methylpyrrolidin-3-ol;
23) 2-(8-((3aS,7aR)-6-methyloctahydro-1H-pyrrolo[2,3-c]pyridin-1-yl)pyrido[2,3-d]pyridazin-5-yl)-5-(trifluoromethyl)phenol;
24) (3S,4s,5R)-1-(5-(2-hydroxy-4-(trifluoromethyl)phenyl)pyrido[2,3-d]pyridazin-8-yl)-3,4,5-trimethylpiperidin-4-ol;
25) 8-(5-(2-hydroxy-4-(trifluoromethyl)phenyl)pyrido[2,3-d]pyridazin-8-yl)-3-oxa-1,8-diazaspiro[4.5]decan-2-one;
26) (cis)-4-(5-(2-hydroxy-4-(trifluoromethyl)phenyl)pyrido[2,3-d]pyridazin-8-yl)-2,6-dimethylthiomorpholine 1,1-dioxide;
27) (R)-1-(5-(2-hydroxy-4-(trifluoromethyl)phenyl)pyrido[2,3-d]pyridazin-8-yl)-3-(trifluoromethyl)pyrrolidin-3-ol;
28) (S)-1-(5-(2-hydroxy-4-(trifluoromethyl)phenyl)pyrido[2,3-d]pyridazin-8-yl)-3-(trifluoromethyl)pyrrolidin-3-ol;
29) (R)-3-(difluoromethyl)-1-(5-(2-hydroxy-4-(trifluoromethyl)phenyl)pyrido[2,3-d]pyridazin-8-yl)pyrrolidin-3-ol;
30) (S)-3-(difluoromethyl)-1-(5-(2-hydroxy-4-(trifluoromethyl)phenyl)pyrido[2,3-d]pyridazin-8-yl)pyrrolidin-3-ol;
31) (R)-2-(8-(1-oxa-7-azaspiro[4.4]nonan-7-yl)pyrido[2,3-d]pyridazin-5-yl)-5-(trifluoromethyl)phenol;
32) (S)-2-(8-(1-oxa-7-azaspiro[4.4]nonan-7-yl)pyrido[2,3-d]pyridazin-5-yl)-5-(trifluoromethyl)phenol;
33) 2-(8-(1-oxa-8-azaspiro[4.5]decan-8-yl)pyrido[2,3-d]pyridazin-5-yl)-5-(trifluoromethyl)phenol;
34) 2-(8-(2-oxa-8-azaspiro[4.5]decan-8-yl)pyrido[2,3-d]pyridazin-5-yl)-5-(trifluoromethyl)phenol;
35) 9-(5-(2-hydroxy-4-(trifluoromethyl)phenyl)pyrido[2,3-d]pyridazin-8-yl)-1-oxa-4,9-diazaspiro[5.5]undecan-3-one;
36) 2-(8-((cis)-4,4-difluoro-3,5-dimethylpiperidin-1-yl)pyrido[2,3-d]pyridazin-5-yl)-5-(trifluoromethyl)phenol;
37) 1-(8-(5-(2-hydroxy-4-(trifluoromethyl)phenyl)pyrido[2,3-d]pyridazin-8-yl)-1,8-diazaspiro[4.5]decan-1-yl)ethan-1-one;
38) 1-(8-(5-(2-hydroxy-4-(trifluoromethyl)phenyl)pyrido[2,3-d]pyridazin-8-yl)-2,8-diazaspiro[4.5]decan-2-yl)ethan-1-one;
39) 8-(5-(2-hydroxy-4-(trifluoro-methyl)phenyl)pyrido[2,3-d]-pyridazin-8-yl)-3-methyl-1-oxa-3,8-diazaspiro[4.5]decan-2-one;
40) 2-(8-(4,4-dimethyl-1-oxa-8-azaspiro[4.5]decan-8-yl)pyrido[2,3-d]pyridazin-5-yl)-5-(trifluoromethyl)phenol;
41) (R)-1-(5-(2-hydroxy-4-(trifluoromethyl)phenyl)pyrido[2,3-d]pyridazin-8-yl)-3-phenylpyrrolidin-3-ol;
42) (S)-1-(5-(2-hydroxy-4-(trifluoromethyl)phenyl)pyrido[2,3-d]pyridazin-8-yl)-3-phenylpyrrolidin-3-ol;
43) 8-(5-(2-hydroxy-4-(trifluoromethyl)phenyl)pyrido[2,3-d]pyridazin-8-yl)-1-methyl-1,8-diazaspiro[4.5]decan-2-one;
44) 8-(5-(2-hydroxy-4-(trifluoromethyl)phenyl)pyrido[2,3-d]pyridazin-8-yl)-1-oxa-3,8-diazaspiro[4.5]decan-2-one;
45) 2-(8-(6,6-difluoro-3-azabicyclo[3.1.0]hexan-3-yl)pyrido[2,3-d]pyridazin-5-yl)-5-(trifluoromethyl)phenol;
46) 8-(5-(2-hydroxy-4-(trifluoromethyl)phenyl)pyrido[2,3-d]pyridazin-8-yl)-1-oxa-8-azaspiro[4.5]decan-2-one;
47) (R)-4,4-difluoro-1-(5-(2-hydroxy-4-(trifluoromethyl)phenyl)pyrido[2,3-d]pyridazin-8-yl)pyrrolidin-3-ol;
48) (S)-4,4-difluoro-1-(5-(2-hydroxy-4-(trifluoromethyl)phenyl)pyrido[2,3-d]pyridazin-8-yl)pyrrolidin-3-ol;
49) (R)-5-(5-(2-hydroxy-4-(trifluoromethyl)phenyl)pyrido[2,3-d]pyridazin-8-yl)-5-azaspiro[2.4]heptan-7-ol;
50) (S)-5-(5-(2-hydroxy-4-(trifluoromethyl)phenyl)pyrido[2,3-d]pyridazin-8-yl)-5-azaspiro[2.4]heptan-7-ol;
51) (R)-3-cyclopropyl-1-(5-(2-hydroxy-4-(trifluoromethyl)phenyl)pyrido[2,3-d]pyridazin-8-yl)pyrrolidin-3-ol;
52) (S)-3-cyclopropyl-1-(5-(2-hydroxy-4-(trifluoromethyl)phenyl)pyrido[2,3-d]pyridazin-8-yl)pyrrolidin-3-ol;
53) (R)-3-((dimethylamino)methyl)-1-(5-(2-hydroxy-4-(trifluoromethyl)phenyl)pyrido-[2,3-d]pyridazin-8-yl)pyrrolidin-3-ol;
54) (S)-3-((dimethylamino)methyl)-1-(5-(2-hydroxy-4-(trifluoromethyl)phenyl)pyrido-[2,3-d]pyridazin-8-yl)pyrrolidin-3-ol;
55) (R)-3-ethyl-1-(5-(2-hydroxy-4-(trifluoromethyl)phenyl)pyrido[2,3-d]pyridazin-8-yl)-pyrrolidin-3-ol;
56) (S)-3-ethyl-1-(5-(2-hydroxy-4-(trifluoromethyl)phenyl)pyrido[2,3-d]pyridazin-8-yl)-pyrrolidin-3-ol;
57) (R)-2-(8-(3-(hydroxymethyl)-3-methylpyrrolidin-1-yl)pyrido[2,3-d]pyridazin-5-yl)-5-(trifluoromethyl)phenol;
58) (S)-2-(8-(3-(hydroxymethyl)-3-methylpyrrolidin-1-yl)pyrido[2,3-d]pyridazin-5-yl)-5-(trifluoromethyl)phenol;
59) (R)-2-(8-(3-(2-hydroxypropan-2-yl)pyrrolidin-1-yl)pyrido[2,3-d]pyridazin-5-yl)-5-(trifluoromethyl)phenol;
60) (S)-2-(8-(3-(2-hydroxypropan-2-yl)pyrrolidin-1-yl)pyrido[2,3-d]pyridazin-5-yl)-5-(trifluoromethyl)phenol;
61) 2-(8-((2R,6R)-2-(hydroxymethyl)-6-methylmorpholino)pyrido[2,3-d]pyridazin-5-yl)-5-(trifluoromethyl)phenol;
62) 2-(8-((2S,6S)-2-(hydroxymethyl)-6-methylmorpholino)pyrido[2,3-d]pyridazin-5-yl)-5-(trifluoromethyl)phenol;
63) 2-(8-((2S,6S)-2-(hydroxymethyl)-6-methylmorpholino)pyrido[2,3-d]pyridazin-5-yl)-5-(trifluoromethyl)phenol;
64) 2-(8-((2R,6R)-2-(hydroxymethyl)-6-methylmorpholino)pyrido[2,3-d]pyridazin-5-yl)-5-(trifluoromethyl)phenol;
65) 1-((2R,4r,6S)-2,6-dimethyltetrahydro-2H-pyran-4-yl)-4-(2-methyl-4(trifluoromethyl)-phenyl)phthalazine;
66) (2R,4s,6S)-4-(5-(2-hydroxy-4-(trifluoromethyl)phenyl)pyrido[2,3-d]-pyridazin-8-yl)-2,6-dimethyltetrahydro-2H-pyran-4-ol;
67) (2R,4r,6S)-4-(5-(2-hydroxy-4-(trifluoromethyl)phenyl)pyrido[2,3-d]-pyridazin-8-yl)-2,6-dimethyltetrahydro-2H-pyran-4-ol;
68) (S)-1-(2-(difluoromethyl)-5-(2-hydroxy-4-(trifluoromethyl)phenyl)pyrido[2,3-d]pyridazin-8-yl)-3-methylpyrrolidin-3-ol; and
69) (S)-1-(3-(difluoromethyl)-5-(2-hydroxy-4-(trifluoromethyl)phenyl)pyrido[2,3-d]pyridazin-8-yl)-3-methylpyrrolidin-3-ol;
   or a pharmaceutically acceptable salt thereof.

Additional illustrative, but non-limiting, examples of compounds of the present invention that are useful as inhibitors of the NLRP3 are the following compounds:
1) (S)-1-(4-(2-hydroxy-4-(trifluoromethyl)phenyl)pyrido[3,4-d]pyridazin-1-yl)-3-methylpyrrolidin-3-ol;
2) (S)-1-(1-(2-hydroxy-4-(trifluoromethyl)phenyl)pyrido[3,4-d]-pyridazin-4-yl)-3-methylpyrrolidin-3-ol;
3) 2-(4-((cis)-2,6-dimethylmorpholino)pyridazino[4,5-d]pyridazin-1-yl)-5-(trifluoromethyl)phenol;
4) (S)-1-(5-(2-hydroxy-4-(trifluoromethyl)phenyl)pyrido[2,3-d]-pyridazin-8-yl)-3-methylpyrrolidin-3-ol;
5) 2-(8-((3aS,7aR)-6-methyloctahydro-1H-pyrrolo[2,3-c]pyridin-1-yl)pyrido[2,3-d]pyridazin-5-yl)-5-(trifluoromethyl)phenol;
6) (2R,4s,6S)-4-(5-(2-hydroxy-4-(trifluoromethyl)phenyl)pyrido[2,3-d]-pyridazin-8-yl)-2,6-dimethyltetrahydro-2H-pyran-4-ol; and
7) (2R,4r,6S)-4-(5-(2-hydroxy-4-(trifluoromethyl)phenyl)pyrido[2,3-d]-pyridazin-8-yl)-2,6-dimethyltetrahydro-2H-pyran-4-ol;
   or pharmaceutically acceptable salts thereof.

Although the specific stereochemistries described above are preferred, other stereoisomers, including diastereoisomers, enantiomers, epimers, and mixtures of these may also have utility in treating NLRP3 mediated diseases.

Synthetic methods for making the compounds are disclosed in the Examples shown below. Where synthetic details are not provided in the examples, the compounds are readily made by a person of ordinary skill in the art of medicinal chemistry or synthetic organic chemistry by applying the synthetic information provided herein. Where a stereochemical center is not defined, the structure represents a mixture of stereoisomers at that center. For such compounds, the individual stereoisomers, including enantiomers, diastereoisomers, and mixtures of these are also compounds of the invention.

### Definitions

“Ac” is acetyl, which is CH₃C(═O)—.

“Alkyl” means saturated carbon chains which may be linear or branched or combinations thereof, unless the carbon chain is defined otherwise. Other groups having the prefix “alk”, such as alkoxy and alkanoyl, also may be linear or branched, or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched, or combinations thereof, unless otherwise defined. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched, or combinations thereof, unless otherwise defined. Examples of alkynyl include ethynyl, propargyl, 3-methyl-1-pentynyl, 2-heptynyl and the like.

“Cycloalkyl” means a saturated monocyclic, bicyclic, spirocyclic, fused or bridged carbocyclic ring, having a specified number of carbon atoms. Examples of cycloalkyl include cyclopropyl, cyclobutyl, cyclopentyl, cyclohexyl, cycloheptyl, and the like. In one embodiment, cycloalkyl is-C₃₋₁₂cycloalkyl. In another embodiment of the present invention, cycloalkyl is selected from: cyclopropyl, cyclobutyl, cyclopentyl, and cyclohexyl. In another embodiment, cycloalkyl is cyclopropyl.

“Cycloalkenyl” means a monocyclic, bicyclic, spirocyclic, fused or bridged carbocyclic ring, having a specified number of carbon atoms with at least one double bond. Examples of cycloalkenyl include cyclopropene, cyclobutene, cyclopentene, cyclohexene, cycloheptene, and the like. In one embodiment, cycloalkenyl is —C₃₋₁₂cycloalkenyl.

“Cycloheteroalkyl” means a saturated monocyclic, bicyclic, spirocyclic, fused or bridged ring or ring system having a specified number of carbon atoms and containing at least one ring heteroatom selected from N, NH, S (including SO and SO₂) and O. The cycloheteroalkyl ring may be substituted on the ring carbons and/or the ring nitrogen or sulfur. Examples of cycloheteroalkyl include tetrahydrofuranyl, pyrrolidinyl, tetrahydrothiophenyl, azetidinyl, piperazinyl, piperidinyl, morpholinyl, oxetanyl and tetrahydropyranyl. In one embodiment of the present invention, cycloheteroalkyl is: C₂₋₁₂cycloheteroalkyl. In another embodiment cycloheteroalkyl is selected from: morpholine, thiomorpholine, piperidine, piperazine, pyrrolidine, tetrahydropyran, octahydro-1H-pyrrolo[2,3-c]pyridine, 3-azabicyclo[3.1.0]hexane, 5-azaspiro[2.4]heptane, 1-oxa-7-azaspiro[4.4]nonane, 1-oxa-8-azaspiro[4.5]decane, 3-oxa-1,8-diazaspiro[4.5]decane, 2,8-diazaspiro[4.5]decane, 1-oxa-3,8-diazaspiro[4.5]decane, 2-oxa-8-azaspiro[4.5]decane, 1,8-diazaspiro[4.5]decane, and 1-oxa-4,9-diazaspiro[5.5]undecane,

“Cycloheteroalkenyl” means a monocyclic, bicyclic, spirocyclic, fused, or bridged ring or ring system having a specified number of carbon atoms and containing at least one double bond and at least one heteroatom selected from N, NH, S (including SO and SO₂) and O. Examples of cycloheteroalkenyl include dihydropyran and dihydrofuran, and the like.

“Aryl” means a monocyclic, bicyclic or tricyclic carbocyclic aromatic ring or ring system containing 6-14 carbon atoms, wherein at least one of the rings is aromatic. Examples of aryl include phenyl and naphthyl. In one embodiment, aryl is phenyl.

“Heteroaryl” means a monocyclic, bicyclic or tricyclic ring or ring system containing 5-14 ring atoms and containing at least one ring heteroatom selected from N, NH, S (including SO and SO₂) and O, wherein at least one of the heteroatom containing rings is aromatic. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzofuranyl, benzothiophenyl, quinolyl, indolyl, isoquinolyl, quinazolinyl, dibenzofuranyl, and the like. In one embodiment, heteroaryl is pyridine or benzofuran. In another embodiment, heteroaryl is pyridine. In another embodiment, heteroaryl is benzofuran.

“Halogen” includes fluorine, chlorine, bromine and iodine. In one embodiment, halogen is fluorine, chorine or bromine. In another embodiment, halogen is fluorine or chlorine. In another embodiment, halogen is chlorine or bromine. In another embodiment, halogen is fluorine or bromine. In another embodiment, halogen is fluorine. In another embodiment, halogen is chlorine. In another embodiment, halogen is bromine.

“Me” represents methyl.

“Oxo” represents ═O.

“Saturated” means containing only single bonds.

“Unsaturated” means containing at least one double or triple bond. In one embodiment, unsaturated means containing at least one double bond. In another embodiment, unsaturated means containing at least one triple bond.

When any variable (e.g., R¹, R^{a}, etc.) occurs more than one time in any constituent or in formula I, its definition on each occurrence is independent of its definition at every other occurrence. Also, combinations of substituents and/or variables are permissible only if such combinations result in stable compounds. A squiggly line across a bond in a substituent variable represents the point of attachment.

Undernomenclature used throughout this disclosure, the point of attachment is described first, followed by the terminal portion of the designated side chain. For example, a C₁₋₅ alkylcarbonylamino C₁₋₆ alkyl substituent is equivalent to:

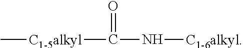

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The phrase “pharmaceutically acceptable” is employed herein to refer to those compounds, materials, compositions, salts and/or dosage forms which are, using sound medical judgment, and following all applicable government regulations, safe and suitable for administration to a human being or an animal.

Compounds of Formula I may contain one or more asymmetric centers and can thus occur as racemates and racemic mixtures, single enantiomers, diastereomeric mixtures and individual diastereomers. The present invention is meant to encompass all such isomeric forms of the compounds of Formula I.

The independent syntheses of optical isomers and diastereoisomers or their chromatographic separations may be achieved as known in the art by appropriate modification of the methodology disclosed herein. Their absolute stereochemistry may be determined by the X-ray crystallography of crystalline products or crystalline intermediates which are derivatized, if necessary, with a reagent containing an asymmetric center of known absolute configuration or sufficient heavy atoms to make an absolute assignment.

If desired, racemic mixtures of the compounds may be separated so that the individual enantiomers are isolated. The separation can be carried out by methods well-known in the art, such as the coupling of a racemic mixture of compounds to an enantiomerically pure compound to form a diastereoisomeric mixture, followed by separation of the individual diastereoisomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula I.

In the compounds of general formula I, the atoms may exhibit their natural isotopic abundances, or one or more of the atoms may be artificially enriched in a particular isotope having the same atomic number, but an atomic mass or mass number different from the atomic mass or mass number predominately found in nature. The present invention is meant to include all suitable isotopic variations of the compounds of structural formula I. For example, different isotopic forms of hydrogen (H) include protium (¹H), deuterium (²H), and tritium (³H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Tritium is radioactive and may therefore provide for a radiolabeled compound, useful as a tracer in metabolic or kinetic studies. Isotopically-enriched compounds within structural formula I, can be prepared without undue experimentation by conventional techniques well known to those skilled in the art or by processes analogous to those described in the Schemes and Examples herein using appropriate isotopically-enriched reagents and/or intermediates.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

### Salts

It will be understood that, as used herein, references to the compounds of the present invention are meant to also include the pharmaceutically acceptable salts, and also salts that are not pharmaceutically acceptable when they are used as precursors to the free compounds or their pharmaceutically acceptable salts or in other synthetic manipulations.

The compounds of the present invention may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the compounds of this invention which are generally prepared by reacting the free base with a suitable organic or inorganic acid. Representative salts of basic compounds of the present invention include, but are not limited to, the following: acetate, benzenesulfonate, benzoate, bicarbonate, bisulfate, bitartrate, borate, bromide, camsylate, carbonate, chloride, clavulanate, citrate, dihydrochloride, edetate, edisylate, estolate, esylate, formic, fumarate, gluceptate, gluconate, glutamate, glycollylars-anilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, oxalate, pamoate (embonate), palmitate, pantothenate, phosphate, diphosphate, polygalacturonate, salicylate, stearate, sulfate, subacetate, succinate, tannate, tartrate, teoclate, tosylate, triethiodide, trifluoroacetate and valerate. Where the compounds of the invention carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, cyclic amines, and basic ion-exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

Also, in the case of a carboxylic acid (—COOH) or alcohol group being present in the compounds of the present invention, pharmaceutically acceptable esters of carboxylic acid derivatives, such as methyl, ethyl, or pivaloyloxymethyl, or acyl derivatives of alcohols, such as O-acetyl, O-pivaloyl, O-benzoyl, and O-aminoacyl, can be employed. Included are those esters and acyl groups known in the art for modifying the solubility or hydrolysis characteristics for use as sustained-release or prodrug formulations.

The term “prodrug” means compounds that are rapidly transformed, for example, by hydrolysis in blood, in vivo to the parent compound, e.g., conversion of a prodrug of Formula I to a compound of Formula I, or to a salt thereof; a thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference. This invention includes prodrugs of the novel compounds of this invention. Solvates, and in particular, the hydrates of the compounds of the present invention are included in the present invention as well.

### Utilities

The compounds of the present invention are potent inhibitors of Nod-Like Receptor Protein 3 (NLPR3). The compounds, and pharmaceutically acceptable salts thereof, may be efficacious in the treatment of diseases, disorders and conditions that are mediated by the inhibition of Nod-Like Receptor Protein 3 (NLPR3).

The present invention relates to the treatment or prevention of a disease, disorder or condition mediated by NLRP3 such as inflammation, an auto-immune disease, a cancer, an infection, a disease or disorder of the central nervous system, a metabolic disease, a cardiovascular disease, a fibrotic disease or fibrosis, a respiratory disease, a kidney disease, a liver disease, an ophthalmic or ocular disease, a skin disease, a lymphatic disease, a rheumatic disease, graft versus host disease, allodynia, or an NLRP3-related disease in a subject that has been determined to carry a germline or somatic non-silent mutation in NLRP3.

The disease, disorder or condition mediated by NLRP3 includes but is not limited to: gout, pseudogout, osteoarthritis, familial cold autoinflammatory syndrome, Muckle-Wells syndrome, neonatal onset multisystem inflammatory disease, diabetes, NASH, sepsis, age related macular degeneration, diabetic retinopathy, liver fibrosis, kidney fibrosis, atherosclerosis, heart failure, peripheral artery disease, myeloproliferative neoplasm, leukemia, myelodysplastic syndrome, myelofibrosis, lung cancer, colon cancer, Parkinson's disease, Alzheimer's disease, traumatic brain injury, spinal cord injury, amyotrophic lateral sclerosis, multiple sclerosis, atopic dermatitis, hidradenitis suppurativa, pericarditis, myocarditis, preeclampsia, dermatomyositis. Still's disease, juvenile idiopathic arthritis, age related macular degeneration, diabetic retinopathy, acute kidney disease, a chronic kidney disease, or a rare kidney disease. Diseases, disorders or conditions mediated by Nod-Like Receptor Protein 3 (NLPR3)), also include, but are not limited to, gout, pseudogout, CAPS, NASH, fibrosis, osteoarthritis, atherosclerosis, heart failure, idiopathic pericarditis, myocarditis, atopic dermatitis, hidradenitis suppurativa, inflammatory bowel disease, cancer, Alzheimer's Disease, Parkinson's Disease and traumatic brain injury.

In one embodiment of the present invention, the condition, disease or disorder is an inflammatory joint disease such as gout, pseudogout, or osteoarthritis.

In another embodiment, the cryopyrin-associated autoinflammatory syndrome is familial cold autoinflammatory syndrome, Muckle-Wells syndrome, or neonatal onset multisystem inflammatory disease.

In another embodiment, the metabolic disease is diabetes.

In another embodiment, the liver disease is NASH.

In another embodiment, the infection is sepsis.

In another embodiment, the ophthalmic or ocular disease is age related macular degeneration or diabetic retinopathy.

In another embodiment, the fibrotic disease is liver fibrosis or kidney fibrosis.

In some embodiments, the cardiovascular disease is atherosclerosis, heart failure, heart failure with preserved ejection fraction or peripheral artery disease.

In another embodiment, the cancer is myeloproliferative neoplasm, leukemia, myelodysplastic syndrome, myelofibrosis, lung cancer or colon cancer.

In another embodiment of the present invention, the condition, disease or disorder of the central nervous system is Parkinson's disease, Alzheimer's disease, traumatic brain injury, spinal cord injury, amyotrophic lateral sclerosis, or multiple sclerosis.

In another embodiment, the skin disease is atopic dermatitis or hidradenitis suppurativa (HS).

In another embodiment, the inflammatory disease is pericarditis or myocarditis.

In another embodiment, the inflammatory disease is preeclampsia.

In another embodiment, the rheumatic disease is dermatomyositis. Still's disease, or juvenile idiopathic arthritis.

In another embodiment, the ocular disease is age related macular degeneration, or diabetic retinopathy.

In another embodiment, the kidney disease is an acute kidney disease, a chronic kidney disease, or a rare kidney disease.

One or more of these conditions or diseases may be treated, managed, prevented, reduced, alleviated, ameliorated or controlled by the administration of a therapeutically effective amount of a compound of the present invention, or a pharmaceutically acceptable salt thereof, to a patient in need of treatment.

The compounds of the present invention may also be used for the manufacture of a medicament which may be useful for treating, preventing, managing, alleviating, ameliorating or controlling one or more of these conditions, diseases or disorders, including but not limited to: gout, pseudogout, osteoarthritis, familial cold autoinflammatory syndrome, Muckle-Wells syndrome, neonatal onset multisystem inflammatory disease, diabetes, NASH, sepsis, age related macular degeneration, diabetic retinopathy, liver fibrosis, kidney fibrosis, atherosclerosis, heart failure, peripheral artery disease, myeloproliferative neoplasm, leukemia, myelodysplastic syndrome, myelofibrosis, lung cancer, colon cancer, Parkinson's disease, Alzheimer's disease, traumatic brain injury, spinal cord injury, amyotrophic lateral sclerosis, multiple sclerosis, atopic dermatitis, hidradenitis suppurativa, pericarditis, myocarditis, preeclampsia, dermatomyositis. Still's disease, juvenile idiopathic arthritis, age related macular degeneration, diabetic retinopathy, acute kidney disease, a chronic kidney disease, or a rare kidney disease. The compounds of the present invention may also be used for the manufacture of a medicament which may be useful for treating, preventing, managing, alleviating, ameliorating or controlling one or more of these conditions, diseases or disorders, including but not limited to: gout, pseudogout, CAPS, NASH, fibrosis, osteoarthritis, atherosclerosis, heart failure, idiopathic pericarditis, myocarditis, atopic dermatitis, hidradenitis suppurativa, inflammatory bowel disease, cancer, Alzheimer's Disease, Parkinson's Disease and traumatic brain injury.

Preferred uses of the compounds may be for the treatment of one or more of the following diseases by administering a therapeutically effective amount to a patient in need of treatment.

The compounds may be used for manufacturing a medicament for the treatment of one or more of these diseases:
1) gout,
2) pseudogout,
3) cryopyrin-associated periodic syndromes,
4) non-alcoholic steatohepatitis,
5) fibrosis,
6) osteoarthritis,
7) atherosclerosis,
8) atopic dermatitis,
9) hidradenitis suppurativa,
10) Alzheimer's Disease, and
11) Parkinson's Disease.

Treatment of a disease, disorder or condition mediated by NLPR3 or the NLPR3 inflammasome pathway refers to the administration of the compounds of the present invention to a subject with the disease, disorder or condition.

One outcome of treatment may be reducing the disease, disorder or condition mediated by NLPR3 or the NLPR3 inflammasome pathway. Another outcome of treatment may be alleviating the disease, disorder or condition mediated by NLPR3 or the NLPR3 inflammasome pathway. Another outcome of treatment may be ameliorating the disease, disorder or condition mediated by NLPR3 or the NLPR3 inflammasome pathway. Another outcome of treatment may be suppressing the disease, disorder or condition mediated by mediated by NLPR3 or the NLPR3 inflammasome pathway. Another outcome of treatment may be managing the disease, disorder or condition mediated by NLPR3 or the NLPR3 inflammasome pathway. Another outcome of treatment may be preventing the disease, disorder or condition mediated by NLPR3 or the NLPR3 inflammasome pathway.

Prevention of the disease, disorder or condition mediated by NLPR3 or the NLPR3 inflammasome pathway refers to the administration of the compounds of the present invention to a subject at risk of the disease, disorder or condition. One outcome of prevention may be reducing the disease, disorder or condition mediated by NLPR3 or the NLPR3 inflammasome pathway in a subject at risk of the disease, disorder or condition. Another outcome of prevention may be suppressing the disease, disorder or condition mediated by NLPR3 or the NLPR3 inflammasome pathway in a subject at risk of the disease, disorder or condition. Another outcome of prevention may be ameliorating the disease, disorder or condition mediated by NLPR3 or the NLPR3 inflammasome pathway in a subject at risk of the disease, disorder or condition. Another outcome of prevention may be alleviating the disease, disorder or condition mediated by NLPR3 or the NLPR3 inflammasome pathway in a subject at risk of the disease, disorder or condition. Another outcome of prevention may be managing the disease, disorder or condition mediated by NLPR3 or the NLPR3 inflammasome pathway in a subject at risk of the disease, disorder or condition.

The terms “administration of” and or “administering a” compound should be understood to mean providing a compound of the invention or a prodrug of a compound of the invention to the individual or mammal in need of treatment.

The administration of the compound of structural formula I in order to practice the present methods of therapy is carried out by administering an effective amount of the compound of structural formula I to the mammal in need of such treatment or prophylaxis. The need for a prophylactic administration according to the methods of the present invention is determined via the use of well known risk factors. The effective amount of an individual compound is determined, in the final analysis, by the physician or veterinarian in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The usefulness of the present compounds in these diseases or disorders may be demonstrated in animal disease models that have been reported in the literature.

### Administration and Dose Ranges

Any suitable route of administration may be employed for providing a mammal, especially a human, with an effective dose of a compound of the present invention. For example, oral, intravenous, infusion, subcutaneous, transcutaneous, intramuscular, intradermal, transmucosal, intramucosal, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of the present invention are administered orally.

In the treatment or prevention of disorders, diseases and/or conditions which require inhibition of NLRP3 a suitable dosage level will generally be about 0.0001 to 500 mg per kg patient body weight per day which can be administered in single or multiple doses. In one embodiment, a suitable dosage level may be about 0.001 to 500 mg per kg patient body weight per day. In another embodiment, a suitable dosage level may be about 0.001 to about 250 mg/kg per day. In another embodiment, a suitable dosage level may be about 0.01 to about 250 mg/kg per day. In another embodiment, a suitable dosage level may be about 0.1 to about 100 mg/kg per day. In another embodiment, a suitable dosage level may be about 0.05 to 100 mg/kg per day. In another embodiment, a suitable dosage level may be about 0.1 to 50 mg/kg per day. In another embodiment, a suitable dosage level may be about 0.05 to 0.5 mg/kg per day. In another embodiment, a suitable dosage level may be about 0.5 to 5 mg/kg per day. In another embodiment, a suitable dosage level may be about 5 to 50 mg/kg per day. For oral administration, the compositions are preferably provided in the form of tablets containing 0.01 to 1000 mg of the active ingredient, particularly 0.01, 0.025, 0.05, 0.075, 0.1, 0.25, 0.5, 0.75, 1.0, 2.5, 5.0, 7.5, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 mg of the active ingredient for the symptomatic adjustment of the dosage to the patient to be treated. The compounds may be administered on a regimen of 1 to 8 times per day; preferably, 1 to 4 times a day; more preferably once or twice per day, even more preferably once a day. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The compounds of this invention may be used in pharmaceutical compositions comprising (a) the compound(s) or pharmaceutically acceptable salts thereof, and (b) a pharmaceutically acceptable carrier. The compounds of this invention may be used in pharmaceutical compositions in which the compound of the present invention or a pharmaceutically acceptable salt thereof is the only active ingredient. The compounds of this invention may also be used in pharmaceutical compositions that include one or more other active pharmaceutical ingredients.

The term “composition,” as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

Compounds of the present invention may be used in combination with other drugs that may also be useful in the treatment or amelioration of the diseases or conditions for which compounds of the present invention are useful. Such other drugs may be administered, by a route and in an amount commonly used therefor, contemporaneously or sequentially with a compound of the present invention. In the treatment of patients who suffer from chronic inflammatory conditions, more than one drug may be administered. The compounds of this invention may generally be administered to a patient who is already taking one or more other drugs for these conditions. Often the compounds will be administered to a patient who is already being treated with one or more anti-pain compounds when the patient's pain is not adequately responding to treatment.

The combination therapy also includes therapies in which the compound of the present invention and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of the present invention.

Examples of other active ingredients that may be administered in combination with a compound of the present invention, and either administered separately or in the same pharmaceutical composition, include but are not limited to:
(i) anti-steatotic agents;
(ii) anti-inflammatory agents;
(iii) immunooncology agent;
(iv) lipid-lowering agents;
(v) cholesterol lowering agents;
(vi) glucose-lowering agents, including SGLT2 inhibitors;
(vii) anti-neovascular agents;
(viii) nonsteroidal anti-inflammatory drugs (“NSAIDs”);
(ix) acetyl-salicylic acid drugs (ASA) including aspirin; paracetamol;
(x) regenerative therapy treatments;
(xi) checkpoint inhibitors including anti-PD1 and anti-PDL1 inhibitors;
(xii) chemotherapy procedures;
(xiii) radiation therapy;
(xiv) surgical procedures;
(xv) urate-lowering therapy;
(xvi) anabolics and cartilage regenerative therapy;
(xvii) anti-fibrotics;
(xviii) JAK inhibitors;
(xix) TNF-alpha inhibitors;
(xx) anti-hypertensive agents; and
(xxi) STING/cGAS antagonists
   pharmaceutically acceptable salts thereof.

In another embodiment of the present invention, the pharmaceutical composition comprises:
1) a compound of Claim 1, or a pharmaceutically acceptable salt thereof;
2) one or more compounds, or pharmaceutically acceptable salts thereof, selected from the group:
   (i) anti-steatotic agents;
   (ii) anti-inflammatory agents;
   (iii) immunooncology agent;
   (iv) lipid-lowering agents;
   (v) cholesterol lowering agents;
   (vi) glucose-lowering agents, including SGLT2 inhibitors;
   (vii) anti-neovascular agents;
   (viii) nonsteroidal anti-inflammatory drugs (“NSAIDs”);
   (ix) acetyl-salicylic acid drugs (ASA) including aspirin; paracetamol;
   (x) regenerative therapy treatments;
   (xi) checkpoint inhibitors including anti-PD1 and anti-PDL1 inhibitors;
   (xii) chemotherapy procedures;
   (xiii) radiation therapy;
   (xiv) surgical procedures;
   (xv) urate-lowering therapy;
   (xvi) anabolics and cartilage regenerative therapy;
   (xvii) anti-fibrotics;
   (xviii) JAK inhibitors;
   (xix) TNF-alpha inhibitors;
   (xx) anti-hypertensive agents; and
   (xxi) STING/cGAS antagonists; and
      pharmaceutically acceptable salts thereof; and
3) a pharmaceutically acceptable carrier.

Specific compounds of use in combination with a compound of the present invention include: anti-steatotic agents, including but not limited to, DGAT2 inhibitors.

Suitable anti-inflammatory agents include, but are not limited to, TNFα inhibitors, JAK inhibitors and NSAIDs.

Suitable lipid-lowering agents include, but are not limited to statins and PCSK9.

Suitable immunooncology agents include, but are not limited to, PD-L1 inhibitors and PD-1 inhibitors and STING antagonists.

Suitable glucose-lowering agents include, but are not limited to, insulin, SGLT2 inhibitors, metformin, GLP1-agonists.

Suitable anti-neovascular agents include, but are not limited to, anti-VEG-F treatment.

Suitable NSAIDs or non-steroidal anti-inflammatory drugs include, but are not limited to, aspirin, diclofenac, diflunisal, etodolac, fenoprofin, flurbiprofen, ibuprofen, indomethacin, ketoprofen, meclofenamic acid, mefenamic acid, meloxicam, naproxen, naproxen sodium, oxaprozin, piroxicam, sulindac, and tolmetin.

Suitable analgesics include, but are not limited to, acetaminophen and duloxetine.

The above combinations include combinations of a compound of the present invention not only with one other active compound, but also with two or more other active compounds. Non-limiting examples include combinations of compounds with two or more active compounds selected from: anti-steatotic agents, anti-inflammatory agents, lipid-lowering agents, anti-fibrosis, immunooncology agents, glucose-lowering agents and anti-neovascular agents, NSAIDs (non-steroidal anti-inflammatory drugs), and an analgesics.

The present invention also provides a method for the treatment or prevention of a NLRP3 mediated disease, disorder or condition, which method comprises administration to a patient in need of such treatment or at risk of developing a NLRP3 mediated disease with a therapeutically effective amount of a NLRP3 inhibitor and an amount of one or more active ingredients, such that together they give effective relief.

In a further aspect of the present invention, there is provided a pharmaceutical composition comprising a NLRP3 inhibitor and one or more active ingredients, together with at least one pharmaceutically acceptable carrier or excipient.

Thus, according to a further aspect of the present invention there is provided the use of a NLRP3 inhibitor and one or more active ingredients for the manufacture of a medicament for the treatment or prevention of an NLRP3-mediated disease, disorder or condition. In a further or alternative aspect of the present invention, there is therefore provided a product comprising a NLRP3 inhibitor and one or more active ingredients as a combined preparation for simultaneous, separate or sequential use in the treatment or prevention of an NLRP3-mediated disease, disorder or condition. Such a combined preparation may be, for example, in the form of a twin pack.

It will be appreciated that for the treatment or prevention of cardiometabolic disease, neurodegenerative disease and inflammatory joint diseases, fibrosis, cancer, a compound of the present invention may be used in conjunction with another pharmaceutical agent effective to treat that disease, disorder or condition.

The present invention also provides a method for the treatment or prevention of chronic inflammatory conditions, which method comprises administration to a patient in need of such treatment an amount of a compound of the present invention and an amount of another pharmaceutical agent effective to threat that disorder, disease or condition, such that together they give effective relief.

The present invention also provides a method for the treatment or prevention of chronic inflammatory conditions, which method comprises administration to a patient in need of such treatment an amount of a compound of the present invention and an amount of another pharmaceutical agent useful in treating that particular condition, disorder or disease, such that together they give effective relief.

The term “therapeutically effective amount” means the amount the compound of structural formula I that will elicit the biological or medical response of a cell, tissue, system, animal or human that is being sought by the researcher, veterinarian, medical doctor or other clinician, which includes alleviation of the symptoms of the disorder being treated. The novel methods of treatment of this invention are for disorders known to those skilled in the art. The term “mammal” includes humans, and companion animals such as dogs and cats.

The weight ratio of the compound of the Formula I to the second active ingredient may be varied and will depend upon the effective dose of each ingredient. Generally, an effective dose of each will be used. Thus, for example, when a compound of the Formula I is combined with an anti-steatotic agent, the weight ratio of the compound of the Formula I generally range from about 1000:1 to about 1:1000, preferably about 200:1 to about 1:200. Combinations of a compound of the Formula I and other active ingredients will generally also be within the aforementioned range, but in each case, an effective dose of each active ingredient should be used.

### Methods of Synthesis

The following reaction schemes and Examples illustrate methods which may be employed for the synthesis of the compounds of structural formula I described in this invention. These reaction schemes and Examples are provided to illustrate the invention and are not to be construed as limiting the invention in any manner. All substituents are as defined above unless indicated otherwise. Several strategies based upon synthetic transformations known in the literature of organic synthesis may be employed for the preparation of the compounds of structural formula I. The scope of the invention is defined by the appended claims. Compound names were generated in Chemdraw Version 21.0.0.28.

### Instrumentation

Reverse phase chromatography was carried out on a Waters 150 equipped with a column selected from the following: Phenomenex Synergi C18 (250 mm×30 mm×4 micron), Phenomenex Luna C18 (250 mm×21 mm×5 micron), Agilent Zorbax Bonus-RP (150 mm×21 mm×5 micron), Waters X-Select CSH C18 (150 mm×19 mm×5 micron). Conditions included either high pH (0-100% acetonitrile/water eluent comprising 0.1% v/v NH4OH) or low pH (0-100% acetonitrile/water eluent comprising 0.1% v/v TFA or Formic Acid) and are noted for some examples. SFC chiral resolution was carried out on Waters Thar 80 SFC or Berger MG II preparative SFC systems using the following conditions: Chiral Method A: ColumnTek Enantiocel A5-5 column, 20% EtOH/CO₂; Chiral Method B: ChiralPak ID column, 35% MeOH/CO₂; Chiral Method C: OJ-H column, 20% iPrOH/CO₂; Chiral Method D: ChiralPak ID column, 5-60% MeOH/CO₂; Chiral Method E: Daicel Chiralpak AD column, 25% iPrOH/CO₂; Chiral Method F: ChiralPak ID column, 30% MeOH/CO₂; Chiral Method G: ChiralPak IB-N column, 15% MeOH/CO₂

LC/MS determinations were carried out on Waters ACQUITY UPLC equipped with a DAD and QDa MS detectors using the following conditions: Waters ACQUITY UPLC BEH C18 1.7 mm 2.1×50 mm column using mobile phase containing A: 0.1% TFA in water and B: 0.1% TFA in acetonitrile with a gradient from 10% B to 90% B over 2.0 min and hold at 90% B for 0.4 min at a flow rate of 0.5 mL/min. Proton or ¹H NMR was acquired using a Bruker 500 MHz NEO NMR spectrometer equipped with a 5 mm iProbe in accordance with standard analytical techniques, unless specified otherwise, and results of spectral analysis are reported. Chemical shift (δ) values are reported in delta (δ) units, parts per million (ppm). Chemical shifts for ¹H NMR spectra are given relative to signals for residual non-deuterated solvent (CDCl₃ referenced at δ 7.26 ppm; DMSO d-6 referenced at δ 2.50 ppm and CD₃OD referenced at δ 3.31 ppm). Multiples are reported by the following abbreviations: s=singlet, d=doublet, t=triplet, q=quartet, dd=doublet of doublets, m=multiplet or overlap of nonequivalent resonances. Coupling constants (J) are reported in Hertz (Hz).

The general preparative conditions of separating diastereomeric or enantiomeric mixtures of compounds using chiral SFC are as follows:

| Chiral | | |
|---|---|---|
| Column | Stationary Phase | Method |
| ColumnTek Enantiocel A5-5 | 20% EtOH/CO₂ | A |
| ChiralPak ID | 35% MeOH/CO₂ | B |
| OJ-H | 20% iPrOH/CO₂ | C |
| ChiralPak ID | 5-60% MeOH/CO₂ | D |
| Daicel Chiralpak AD | 25% iPrOH/CO₂ | E |
| ChiralPak ID | 30% MeOH/CO₂ | F |
| ChiralPak IB-N | 15% MeOH/CO₂ | G |

### Abbreviations

“*” in a molecule designates a stereocenter; Ac is acetyl; OAc is acetate; AcOH is acetic acid; aq. is aqueous; B₂pin₂ is bis(pinacolato)diboron; BPin ester is boronic acid pinacol ester; Boc or boc is tert-butoxycarbonyl; br is broad; Bu or nBu is n-butyl; Bz is benzoyl; ^{□}C is degrees Celsius; Calc'd is calculated; CDI is 1,1′-carbonyl-diimidazole, □ is chemical shift; d is doublet; conc. is concentrated; DFMS is Bis(((difluoromethyl)sulfinyl)oxy)zinc; DIC is N,N′-diisopropyl-carbodiimide; DAST is diethylaminosulfur trifluoride; DCE is dichloroethane; DCM is dichloromethane; dd is doublet of doublets; dqd is doublet of a quartet of doublets; DEA is diethanolamine; DIEA is N,N-diisopropylethylamine; DMA is dimethylacetamide; DME is dimethoxyethane; DMF is dimethylformamide; DMSO is dimethylsulfoxide; dtbbpy is 4,4′-di-tert-butyl-2,2′-dipyridyl; dppf is 1,1′-bis(diphenylphosphino)-ferrocene; EDC is 1-ethyl-3-(3-dimethylaminopropyl)-carbodiimide; ESI is electrospray ionization; Et is ethyl; Et₂O is diethyl ether; EtOAc is ethyl acetate; EtOH is ethanol; equiv is equivalent(s); g is grams; h or hr(s) is hour(s); HOAt is 1-Hydroxy-7-azabenzotriazole; HPLC is high-performance liquid chromatography; Hz is hertz; ⁱPr is isopropyl; ⁱPrOH or IPA is isopropyl alcohol; iPrMgCl is isopropylmagnesium chloride; J is coupling constant; L is liter; LAH is lithium aluminum hydride; LC is liquid chromatography; LCMS is liquid chromatography/mass spectrometry; LRMS is low resolution mass spectrometry; m is multiplet; M is molar; Me is methyl; MeOH is methanol; MeCN is acetonitrile; mg is milligrams; min is minutes; mL is milliliter; mM is millimolar; mmol is millimole(s); MHz is megahertz; Q L is microliter; MS is mass spectrometry; nM is nanomolar; NHPI is N-hydroxyphthalimide; NaHMDS is Sodium bis(trimethylsilyl)amide; NH₄OAc is ammonium acetate, NMO is 4-Methylmorpholine N-oxide; NMP is N-methylpyrrolidone; PCC is pyridinium chlorochromate; Pd/C is palladium on carbon; Pd(dppf)Cl₂ is [1,1′-bis-(diphenylphosphino)-ferrocene]dichloropalladium(II); Pd(PPh₃)₄ is tetrakis(triphenyl-phosphine)palladium(0); Pd(tBu₃P)₂ is Bis(tri-tert-butylphosphine)-palladium(0); PE is petroleum ether; PG is protecting group; ph is phenyl; pr is propyl; prep is preparative; q is quartet; rac is racemic mixture; rt or RT is room temperature; s is singlet; sat. or satd is saturated; SFC is Supercritical Fluid Chromatography; SnAr is nucleophilic aromatic substitution; t is triplet; TBHP is tert-butyl hydroperoxide; ^{t}Bu is tert-butyl; ^{t}BuOH is tert-butyl alcohol; tert is tertiary; TBAF is tetrabutylammonium fluoride; TEA is triethylamine; TFA is trifluoroacetic acid; THF is tetrahydrofuran; Ti(OEt)₄ is titanium (IV) ethoxide; Ti(OiPr)₄ is titanium (IV) isopropoxide; TLC is thin layer chromatography; TMHD is 2,2,6,6-tetramethyl-3,5-heptanedione; TMS-Diazomethane is trimethylsilyl-diazomethane; tt is triplet of triplets; XPhos Pd G3 is (2-Dicyclohexylphosphino-2′,4′,6′-triisopropyl-1,1′-biphenyl)[2-(2′-amino-1,1′-biphenyl)]palladium(II) methanesulfonate; and UV is ultraviolet.

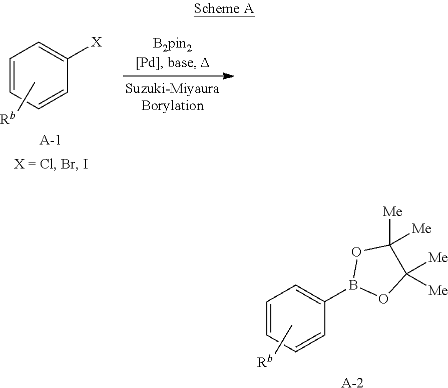

Scheme A illustrates the synthesis of aryl and heteroaryl boronates such as A-2 from aryl halides (Cl, Br or I) such as A-1. Aryl boronates A-2 are prepared via palladium-catalyzed Suzuki-Miyaura borylation of aryl halides such as A-1 with B₂pin₂ in the presence of suitable base.

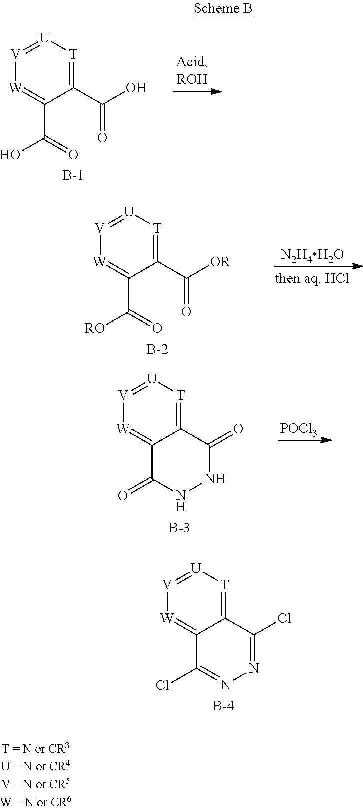

Scheme B illustrates the synthetic sequence for the preparation of phthalazine derivatives of the formula B-4. Heteroaryl diacids of the formula B-1 are treated with acid, such as H₂SO₄, in a suitable alcoholic solvent (ROH), such as MeOH, to afford diesters of the formula B-2. Diesters of the formula B-2 are condensed with hydrazine followed by treatment with aq. HCl to give pyridazine-1,4-diones of the formula B-3. The reaction of pyridazine-1,4-diones B-3 with a suitable deoxychlorination reagent (such as POCl₃) gives dichlorophthalazine derivatives of the formula B-4.

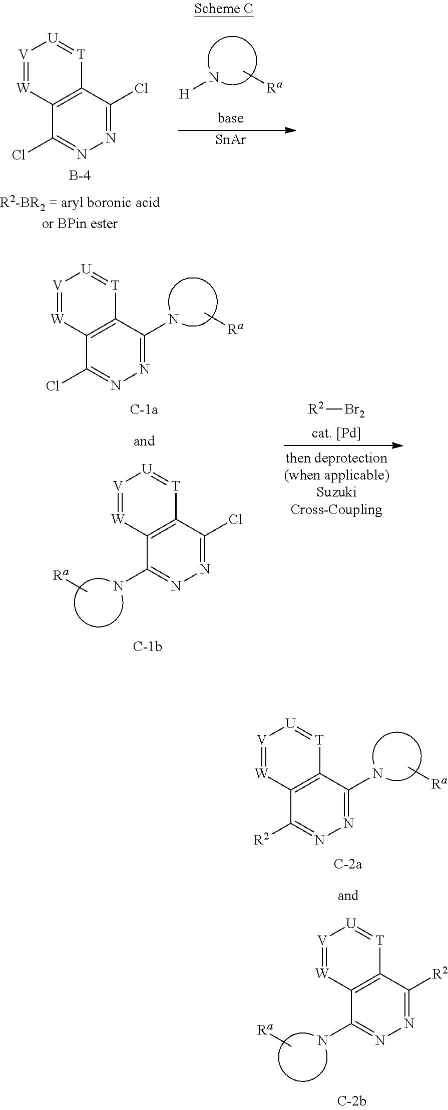

Scheme C illustrates a synthetic sequence for preparation of phthalazine derivatives such as C-2a and C-2b from precursors of the formula B-4. Nucleophilic aromatic substitution (SnAr) proceeds using various secondary amines in the presence of base, such as K₂CO₃, to afford tertiary amines of the formulas C-1a and C-1b. Cross coupling using an appropriate aryl nucleophile, such as aryl boronic acid, and palladium catalyst gives biaryl products which are deprotected in situ, when applicable, to afford compounds of the formula C-2a and C-2b. Regioisomers, if present, are separated chromatographically, preferably using reverse phase HPLC or SFC.

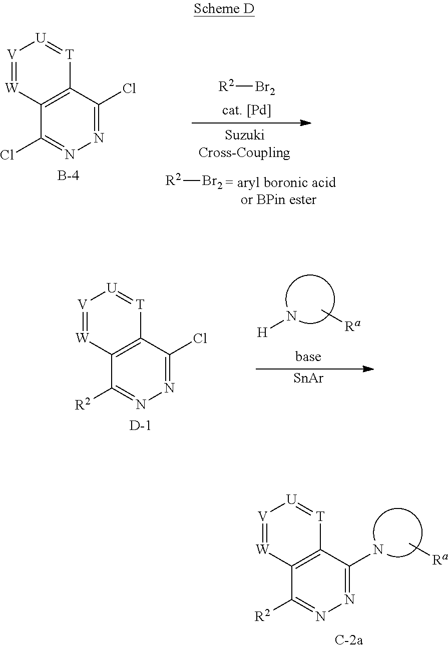

Scheme D illustrates a synthetic sequence or preparation of phthalazine derivatives such as C-2a from precursors of the formula B-4. Suzuki cross coupling using an appropriate aryl nucleophile, such as aryl boronic acid, and palladium catalyst gives biaryl products of the formula D-1; regioisomers, if present, are separated chromatographically, preferably using silica gel chromatography. Subsequent nucleophilic aromatic substitution (SnAr) using various secondary amines in the presence of base, such as K₂CO₃, delivers tertiary amines of the formula C-2a.

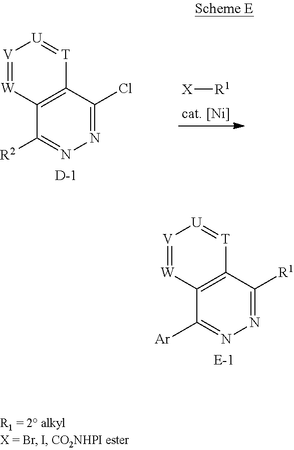

Scheme E illustrates the method to prepare alkylated compounds of the formula E-1 from the corresponding biaryl chloride D-1 and appropriate alkyl electrophile, such as alkyl halide or N-hydroxyphthalimide (NHPI) ester. Cross-electrophile coupling proceeds under nickel catalysis, to afford alkylated compounds of the formula E-1.

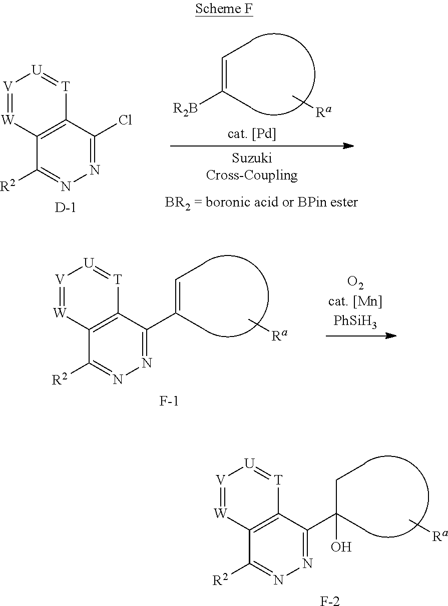

Scheme F illustrates the method to prepare tertiary alcohol compounds of the formula F-2 from the corresponding biaryl chloride D-1 and an appropriate alkenyl nucleophile, such as pinacol boronate ester. Cross coupling proceeds under palladium catalysis to afford biaryl alkenes of the formula F-1. Subsequent alkene hydration using Mukaiyama-Magnus conditions proceeds to deliver tertiary alcohols of the formula F-2.

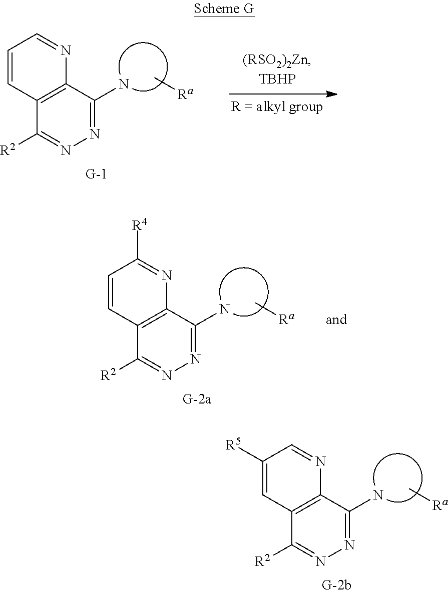

Scheme G illustrates the method to prepare alkylated pyridopyridazines of the formula G-2a and G-2b via Minisci-type radical addition to G-1 using suitable radical precursors, such as zinc sulfinate reagents. The reaction proceeds in the presence of an oxidant, such as TBHP, to afford regioisomeric mixtures of substituted pyridopyridazines of the formula G-2a and G-2b, which are subsequently separated by SFC.

### Intermediate 1

### 2-(2-(difluoromethoxy)-4-(trifluoromethyl)phenyl)-4,4,5,5-tetramethyl-1,3,2-dioxaborolane

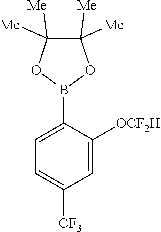

To a solution of 1-bromo-2-(difluoromethoxy)-4-(trifluoromethyl)benzene (Enamine, 60 mg, 0.2 mmol) in 1,4-dioxane (1.0 mL) were added B₂pin₂ (56 mg, 0.22 mmol), KOAc (60 mg, 0.6 mmol) and PdCl₂(dppf) (15 mg, 0.2 mmol). The mixture was degassed with N₂ and then stirred at 105° C. for 12 h. The reaction mixture was then cooled to room temperature, filtered, and the solvents removed under vacuum. The resulting crude mixture was carried forward without further purification. ¹H NMR (500 MHz, CDCl₃) δ 7.88 (br d, J=7.6 Hz, 1H), 7.51 (br d, J=7.7 Hz, 1H), 7.41 (s, 1H), 6.26-6.82 (t, J=74.4 Hz, 1H), 1.37 ppm (s, 12H).

### Intermediate 2

### 1,4-dichloropyridazino[4,5-d]pyridazine

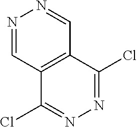 [SMILES: Clc1nnc(Cl)c2cnncc12]

Step 1: dimethyl pyridazine-4,5-dicarboxylate: A solution of pyridazine-4,5-dicarboxylic acid (Combi-Blocks, 2.0 g, 11.9 mmol) in MeOH (40 mL) was treated with concentrated H₂SO₄ (0.64 mL, 11.9 mmol). The mixture was heated to reflux for 48 h, then cooled to room temperature and partitioned between H₂O and EtOAc. The aqueous layer was extracted with EtOAc, and the combined organic layers were washed with brine, dried with Na₂SO₄, filtered, and concentrated under reduced pressure. The resulting crude residue was purified by silica gel chromatography (EtOAc:hexanes) to give the title compound. LCMS [M+H]⁺=197.1 (calcd. 197.1).

Step 2: 2,3-dihydropyridazino[4,5-d]pyridazine-1,4-dione: A solution of dimethyl pyridazine-4,5-dicarboxylate (578 mg, 2.95 mmol) in MeOH (7.3 mL) was treated with hydrazine hydrate (0.44 mL, 8.84 mmol) at room temperature, and then heated to reflux for 1 h with vigorous stirring. The mixture was cooled to room temperature and filtered. The filtered solid was transferred to a vial containing H₂O (9 mL) and heated to 85° C.; then the mixture was acidified to pH 3 with concentrated aqueous HCl, and stirred for 20 min. The mixture was cooled to room temperature, filtered, and the resulting solid was dried under reduced pressure to afford the title compound. LCMS [M+H]⁺=165.0 (calcd. 165.0).

Step 3: 1,4-dichloropyridazino[4,5-d]pyridazine: A solution of 2,3-dihydropyridazino[4,5-d]pyridazine-1,4-dione (100 mg, 0.61 mmol) and PCl₅ (Sigma Aldrich, 254 mg, 1.22 mmol) in POCl₃ (Sigma Aldrich, 1.4 mL, 15.2 mmol) was heated to 110° C. overnight. The mixture was cooled to room temperature and poured onto ice. The suspension was adjusted to pH 7 with saturated aq. NaHCO₃, and then diluted with EtOAc. The layers were separated, and the aqueous layer was extracted with EtOAc (×2). The combined organic layers were dried over Na₂SO₄, filtered, and concentrated under reduced pressure. The crude residue was purified by silica gel chromatography (EtOAc:hexanes) to afford the title compound. LCMS [M+H]⁺=201.0 (calcd. 201.0).

TABLE 1
The following compounds were prepared using a procedure
similar to that described for Intermediate 2 using the
appropriate starting materials.

| | | | LCMS |
|---|---|---|---|
| Intermediate | Structure | Name | [M + H]⁺ |
| 3 | 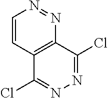 [SMILES: Clc1nnc(Cl)c2nnccc12] | 5,8-dichloropyridazino [4,5-c]pyridazine | Calcd. 201.0, found 201.0 |
| 4 | 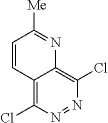 [SMILES: Cc1ccc2c(Cl)nnc(Cl)c2n1] | 5,8-dichloro-2- methylpyrido[2,3-d] pyridazine | Calcd. 214.0, found 213.9 |
| 5 | 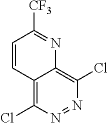 [SMILES: FC(F)(F)c1ccc2c(Cl)nnc(Cl)c2n1] | 5,8-dichloro-2- (trifluoromethyl)pyrido [2,3-d]pyridazine | Calcd. 268.0, found 267.9 |

### Intermediate 6

### (3aR,7aR)-6-methyloctahydro-1H-pyrrolo[2,3-c]pyridine

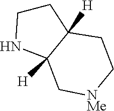

Step 1: tert-butyl (3aS,7aR)-octahydro-1H-pyrrolo[2,3-c]pyridine-1-carboxylate: Racemic tert-butyl (cis)-octahydro-1H-pyrrolo[2,3-c]pyridine-1-carboxylate (Synthonix, 15 g, 66.3 mmol) was separated by chiral SFC (Method A) to afford the corresponding enantiomers. Peak 1: tert-butyl (3aR,7aS)-octahydro-1H-pyrrolo[2,3-c]pyridine-1-carboxylate (6.8 g, 30.0 mmol), and peak 2: tert-butyl (3aS,7aR)-octahydro-1H-pyrrolo[2,3-c]pyridine-1-carboxylate (5.7 g, 25.2 mmol).

Step 2: tert-butyl (3aS,7aR)-6-methyloctahydro-1H-pyrrolo[2,3-c]pyridine-1-carboxylate: To a solution of tert-butyl (3aS,7aR)-octahydro-1H-pyrrolo[2,3-c]pyridine-1-carboxylate (1 g, 4.42 mmol) in MeOH (8.8 mL) at 0° C. were added 37% aq. formaldehyde (1.65 mL, 22.1 mmol) and NaBH₃CN (0.56 g, 8.84 mmol). The mixture was allowed to warm to room temperature and stirred overnight. The solvents were removed under reduced pressure and the crude residue containing the title compound was used directly in the next step. LCMS [M+H]⁺=241.1 (calcd. 241.2).

Step 3: (3aR,7aR)-6-methyloctahydro-1H-pyrrolo[2,3-c]pyridine: HCl (4 M in 1,4-dioxane, 5.5 ml, 22.0 mmol) was added to a solution of crude tert-butyl (3aS,7aR)-6-methyl-octahydro-1H-pyrrolo[2,3-c]pyridine-1-carboxylate (1.06 g, 4.4 mmol) in 1,4-dioxane (6 mL) and MeOH (3 mL). The mixture was allowed to stir at room temperature overnight. The solvents were removed under reduced pressure and the crude residue containing the title compound was used directly in the next step. LCMS [M+H]⁺=141.1 (calcd. 141.1).

### Intermediate 7

### Cis-1,3-dioxoisoindolin-2-yl 2,6-dimethyltetrahydro-2H-pyran-4-carboxylate

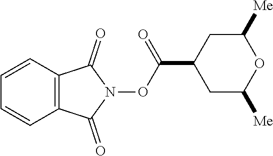 [SMILES: C[C@@H]1C[C@H](C(=O)ON2C(=O)c3ccccc3C2=O)C[C@H](C)O1]

Cis-2,6-dimethyltetrahydro-2H-pyran-4-carboxylic acid (Enamine, 100 mg, 0.63 mmol), N-hydroxyphthalimide (113 mg, 0.7 mmol), and DMAP (3.9 mg, 0.03 mmol) was suspended in DCM (3.1 mL). A stock solution of DIC (1 M in DCM, 0.7 mL, 0.7 mmol) was added, and the mixture was stirred at room temperature overnight. The reaction was filtered, solvents removed under reduced pressure, and the crude residue purified by silica gel chromatography (EtOAc:hexanes) to afford the title compound.

### Examples 1 and 2

### (S)-1-(4-(2-hydroxy-4-(trifluoromethyl)phenyl)pyrido[3,4-d]pyridazin-1-yl)-3-methylpyrrolidin-3-ol (Example 1); and (S)-1-(1-(2-hydroxy-4-(trifluoromethyl)phenyl)pyrido[3,4-d]-pyridazin-4-yl)-3-methylpyrrolidin-3-ol (Example 2)

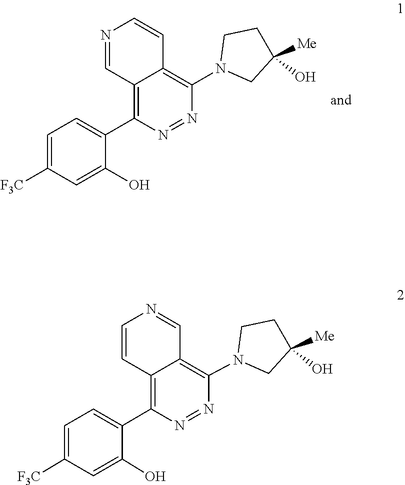 [SMILES: C[C@]1(O)CCN(c2nnc(-c3ccc(C(F)(F)F)cc3O)c3cnccc23)C1 ; "and" ; C[C@]1(O)CCN(c2nnc(-c3ccc(C(F)(F)F)cc3O)c3ccncc23)C1]

Step 1: (S)-1-(4-chloropyrido[3,4-d]pyridazin-1-yl)-3-methylpyrrolidin-3-ol and (S)-1-(1-chloropyrido[3,4-d]pyridazin-4-yl)-3-methylpyrrolidin-3-ol: 1,4-dichloropyrido[3,4-d]pyridazine (Ambeed, 300 mg, 1.5 mmol), K₂CO₃ (415 mg, 3.0 mmol), and (S)-3-methylpyrrolidin-3-ol hydrochloride (PharmaBlock, 206 mg, 1.5 mmol) were suspended in NMP (6 mL). The mixture was heated to 80° C. overnight. Then the reaction was cooled to room temperature, diluted with EtOAc and washed with water. The aqueous layer was extracted with EtOAc, and the combined organic layers were dried with Na₂SO₄, filtered, and concentrated under reduced pressure. The crude residue containing the title compounds as a mixture of regioisomers was used directly in the next step. LCMS [M+H]⁺=265.0 (calcd. 265.1).

Step 2: (S)-1-(4-(2-hydroxy-4-(trifluoromethyl)phenyl)pyrido[3,4-d]pyridazin-1-yl)-3-methylpyrrolidin-3-ol and (S)-1-(1-(2-hydroxy-4-(trifluoromethyl)phenyl)pyrido[3,4-d]pyridazin-4-yl)-3-methylpyrrolidin-3-ol: A mixture of regioisomers (S)-1-(4-chloropyrido[3,4-d]pyridazin-1-yl)-3-methylpyrrolidin-3-ol and (S)-1-(1-chloropyrido[3,4-d]pyridazin-4-yl)-3-methylpyrrolidin-3-ol (555 mg, 2.10 mmol), (2-hydroxy-4-(trifluoromethyl)phenyl)boronic acid (PharmaBlock, 734 mg, 3.56 mmol), and XPhos Pd G3 (Sigma, 177 mg, 0.21 mmol) was suspended in 1,4-dioxane (10 mL) and a stock solution of K₃PO₄ (3 M aqueous solution, 1.54 mL, 4.61 mmol). The mixture was degassed by sparging with Ar for 10 min, then sealed and heated to 100° C. for 3 h with stirring. After cooling to room temperature, the crude mixture was dry loaded on silica gel and purified by silica gel chromatography (MeOH:DCM) to afford the title compounds as mixture of regioisomers. The regioisomers were separated by chiral SFC (Method B). The faster eluting regioisomer was obtained (Example 1). ¹H NMR (500 MHz, DMSO-d₆) δ 8.87 (d, J=5.9 Hz, 1H), 8.83 (s, 1H), 8.18 (d, J=5.9 Hz, 1H), 7.63 (d, J=7.8 Hz, 1H), 7.35 (d, J=7.9 Hz, 1H), 7.30 (s, 1H), 4.94 (s, 1H), 4.20-4.11 (m, 1H), 3.96-3.85 (m, 2H), 3.72 (d, J=10.8 Hz, 1H), 2.04-1.91 (m, 2H), 1.42 (s, 3H). LCMS [M+H]⁺=391.0 (calcd. 391.1). The slower eluting regioisomer was obtained (Example 2). ¹H NMR (500 MHz, DMSO-d₆) δ 10.52 (s, 1H), 9.65 (s, 1H), 8.82 (d, J=5.6 Hz, 1H), 7.58 (d, J=7.7 Hz, 1H), 7.33-7.27 (m, 3H), 4.92 (s, 1H), 4.19 (td, J=10.1, 7.1 Hz, 1H), 3.98 (d, J=11.1 Hz, 1H), 3.97-3.91 (m, 1H), 3.76 (d, J=11.2 Hz, 1H), 2.06-1.92 (m, 2H), 1.43 (s, 3H). LCMS [M+H]⁺=391.0 (calcd. 391.1).

TABLE 2
The following compounds were prepared using a procedure similar to that described for
Examples 1 and 2 using the appropriate starting materials. Regioisomers (if present) were
separated by silica gel chromatography after step 1 or reverse phase HPLC after step 2.

| | | | LCMS |
|---|---|---|---|
| Example | Structure | Name | [M + H]⁺ |
| 3 | 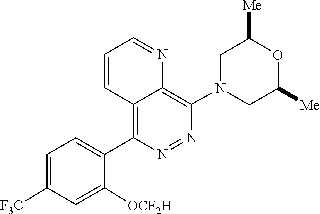 | (cis)-4-(5-(2-(difluoromethoxy)-4- (trifluoromethyl)phenyl)pyrido[2,3- d]pyridazin-8-yl)-2,6- dimethylmorpholine | Calcd. 455.2, found 455.2 |
| 4 | 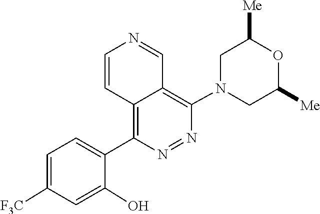 [SMILES: C[C@@H]1CN(c2nnc(-c3ccc(C(F)(F)F)cc3O)c3ccncc23)C[C@H](C)O1] | 2-(4-((cis)-2,6- dimethylmorpholino)pyrido[3,4- d]pyridazin-1-yl)-5- (trifluoromethyl)phenol | Calcd. 405.2, found 405.3 |
| 5 | 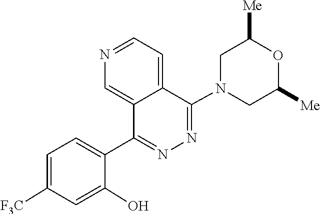 [SMILES: C[C@@H]1CN(c2nnc(-c3ccc(C(F)(F)F)cc3O)c3cnccc23)C[C@H](C)O1] | 2-(1-((cis)-2,6- dimethylmorpholino)pyrido[3,4- d]pyridazin-4-yl)-5- (trifluoromethyl)phenol | Calcd. 405.2, found 405.3 |
| 6 | 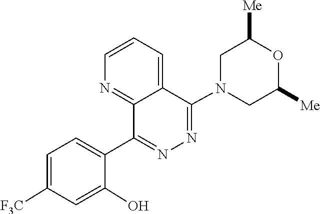 [SMILES: C[C@@H]1CN(c2nnc(-c3ccc(C(F)(F)F)cc3O)c3ncccc23)C[C@H](C)O1] | 2-(5-((cis)-2,6- dimethylmorpholino)pyrido[2,3- d]pyridazin-8-yl)-5- (trifluoromethyl)phenol | Calcd. 405.2, found 405.3 |
| 7 | 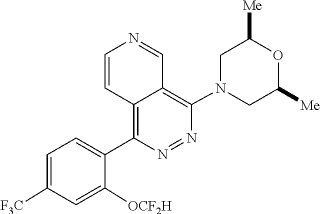 | (cis)-4-(1-(2-(difluoromethoxy)-4- (trifluoromethyl)phenyl)pyrido[3,4- d]pyridazin-4-yl)-2,6- dimethylmorpholine | Calcd. 455.2, found 455.2 |
| 8 | 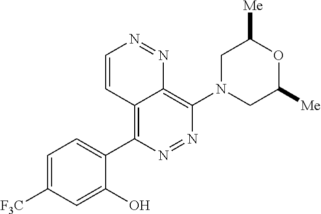 [SMILES: C[C@@H]1CN(c2nnc(-c3ccc(C(F)(F)F)cc3O)c3ccnnc23)C[C@H](C)O1] | 2-(8-((cis)-2,6- dimethylmorpholino)pyridazino[4,5- c]pyridazin-5-yl)-5- (trifluoromethyl)phenol | Calcd. 406.2, found 406.0 |
| 9 | 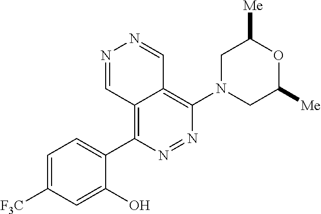 [SMILES: C[C@@H]1CN(c2nnc(-c3ccc(C(F)(F)F)cc3O)c3cnncc23)C[C@H](C)O1] | 2-(4-((cis)-2,6- dimethylmorpholino)pyridazino[4, 5-d]pyridazin-1-yl)-5- (trifluoromethyl)phenol | Calcd. 406.2, found 406.6 |
| 10 | 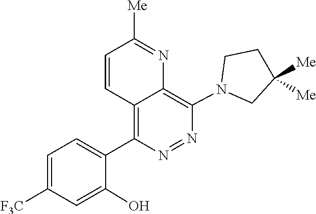 | (S)-1-(5-(2-hydroxy-4- (trifluoromethyl)phenyl)-2- methylpyrido[2,3-d]pyridazin-8- yl)-3-methylpyrrolidin-3-ol | Calcd. 405.2, found 405.2 |
| 11 | 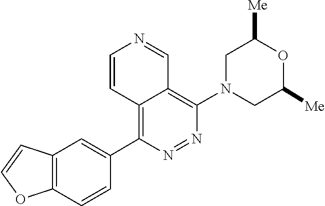 [SMILES: C[C@@H]1CN(c2nnc(-c3ccc4occc4c3)c3ccncc23)C[C@H](C)O1] | (cis)-4-(1-(benzofuran-5- yl)pyrido[3,4-d]pyridazin-4-yl)- 2,6-dimethylmorpholine | Calcd. 361.2, found 361.2 |
| 12 | 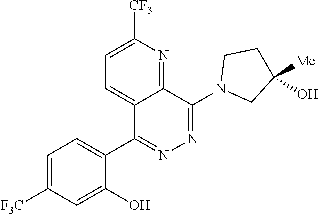 [SMILES: C[C@]1(O)CCN(c2nnc(-c3ccc(C(F)(F)F)cc3O)c3ccc(C(F)(F)F)nc23)C1] | (S)-1-(5-(2-hydroxy-4- (trifluoromethyl)phenyl)-2- (trifluoromethyl)pyrido[2,3- d]pyridazin-8-yl)-3- methylpyrrolidin-3-ol | Calcd. 459.1, found 459.0 |
| 13 | 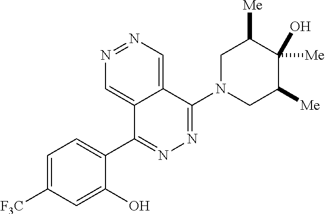 [SMILES: C[C@@H]1CN(c2nnc(-c3ccc(C(F)(F)F)cc3O)c3cnncc23)C[C@H](C)[C@]1(C)O] | (3S,4s,5R)-1-(4-(2-hydroxy-4- (trifluoromethyl)phenyl)pyridazino [4,5-d]pyridazin-1-yl)-3,4,5- trimethylpiperidin-4-ol | Calcd. 434.2, found 434.1 |
| 14 | 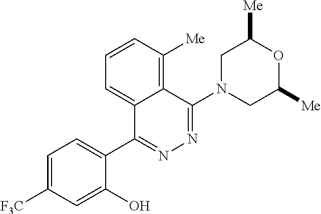 [SMILES: Cc1cccc2c(-c3ccc(C(F)(F)F)cc3O)nnc(N3C[C@@H](C)O[C@@H](C)C3)c12] | 2-(4-((cis)-2,6- dimethylmorpholino)-5- methylphthalazin-1-yl)-5- (trifluoromethyl)phenol | Calcd. 418.2, found 418.2 |
| 15 | 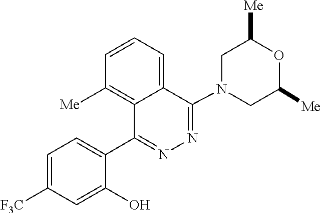 [SMILES: Cc1cccc2c(N3C[C@@H](C)O[C@@H](C)C3)nnc(-c3ccc(C(F)(F)F)cc3O)c12] | 2-(4-((cis)-2,6- dimethylmorpholino)-8- methylphthalazin-1-yl)-5- (trifluoromethyl)phenol | Calcd. 418.2, found 418.1 |

TABLE 3
The following compounds were prepared using procedures similar to those described for
Examples 1 and 2 using the appropriate starting materials. Regioisomeric products were
separated after step 2 using chiral SFC methods specified in the table. For pairs of
regioisomers, the faster-eluting isomer is listed first.

| | | | LCMS | Chiral |
|---|---|---|---|---|
| Example | Structure | Name | [M + H]⁺ | Method |
| 16 | 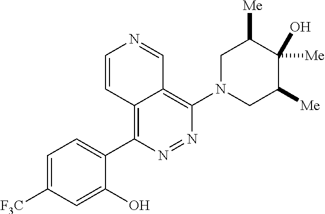 [SMILES: C[C@@H]1CN(c2nnc(-c3ccc(C(F)(F)F)cc3O)c3ccncc23)C[C@H](C)[C@]1(C)O] | (3S,4s,5R)-1-(1-(2- hydroxy-4- (trifluoromethyl)phenyl) pyrido[3,4- d]pyridazin-4-yl)- 3,4,5- trimethylpiperidin-4- ol | Calcd. 433.2, found 433.1 | C |
| 17 | 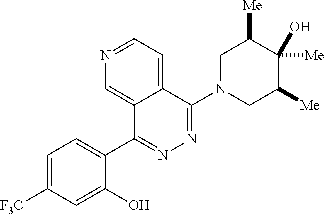 [SMILES: C[C@@H]1CN(c2nnc(-c3ccc(C(F)(F)F)cc3O)c3cnccc23)C[C@H](C)[C@]1(C)O] | (3S,4s,5R)-1-(4-(2- hydroxy-4- (trifluoromethyl)phenyl) pyrido[3,4- d]pyridazin-1-yl)- 3,4,5- trimethylpiperidin-4- ol | Calcd. 433.2, found 433.1 | C |
| 18 | 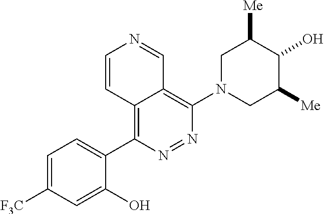 [SMILES: C[C@@H]1CN(c2nnc(-c3ccc(C(F)(F)F)cc3O)c3ccncc23)C[C@H](C)[C@H]1O] | (3S,4r,5R)-1-(1-(2- hydroxy-4- (trifluoromethyl)phenyl) pyrido[3,4- d]pyridazin-4-yl)- 3,5- dimethylpiperidin-4- ol | Calcd. 419.2 found 419.1 | D |
| 19 | 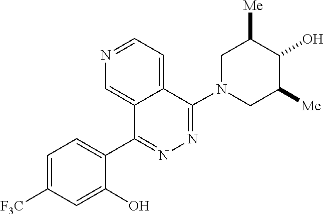 [SMILES: C[C@@H]1CN(c2nnc(-c3ccc(C(F)(F)F)cc3O)c3cnccc23)C[C@H](C)[C@H]1O] | (3S,4r,5R)-1-(4-(2- hydroxy-4- (trifluoromethyl)phenyl) pyrido[3,4- d]pyridazin-1-yl)- 3,5- dimethylpiperidin-4- ol | Calcd. 419.2 found 419.1 | D |
| 20 | 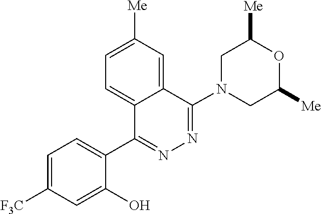 [SMILES: Cc1ccc2c(-c3ccc(C(F)(F)F)cc3O)nnc(N3C[C@@H](C)O[C@@H](C)C3)c2c1] | 2-(4-((cis)-2,6- dimethylmorpholino)- 6-methylphthalazin- 1-yl)-5- (trifluoromethyl)phenol | Calcd. 418.2 found 418.2 | E |
| 21 | 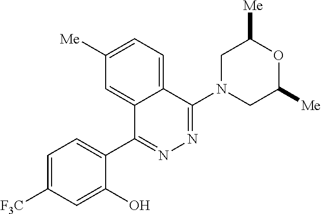 [SMILES: Cc1ccc2c(N3C[C@@H](C)O[C@@H](C)C3)nnc(-c3ccc(C(F)(F)F)cc3O)c2c1] | 2-(4-((cis)-2,6- dimethylmorpholino)- 7-methylphthalazin- 1-yl)-5- (trifluoromethyl)phenol | Calcd. 418.2 found 418.2 | E |

### Example 22

### (S)-1-(5-(2-hydroxy-4-(trifluoromethyl)phenyl)pyrido[2,3-d]-pyridazin-8-yl)-3-methylpyrrolidin-3-ol

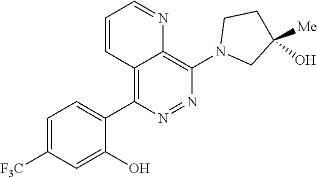 [SMILES: C[C@]1(O)CCN(c2nnc(-c3ccc(C(F)(F)F)cc3O)c3cccnc23)C1]

Step 1: 2-(8-chloropyrido[2,3-d]pyridazin-5-yl)-5-(trifluoromethyl)phenol: A solution of (2-hydroxy-4-(trifluoromethyl)phenyl)boronic acid (PharmaBlock, 2.47 g, 12.0 mmol), 5,8-dichloropyrido[2,3-d]pyridazine (Ambeed, 2 g, 10.0 mmol), K₂CO₃ (2.07 g, 15.0 mmol), and Pd(dppf)Cl₂ (0.48 g, 0.65 mmol) in 1,4-dioxane (30 mL) and H₂O (10 mL) was degassed by sparging with Ar for 10 min. The vial was heated to 85° C. and stirred for 2 h. The reaction was then cooled to room temperature, diluted with EtOAc and dry loaded on silica gel. Purification by silica gel chromatography (EtOAc:hexanes) afforded the title compound. LCMS [M+H]⁺=326.0 (calcd. 326.0).

Step 2: (S)-1-(5-(2-hydroxy-4-(trifluoromethyl)phenyl)pyrido[2,3-d]pyridazin-8-yl)-3-methylpyrrolidin-3-ol: K₂CO₃ (151 mg, 1.09 mmol) and (S)-3-methylpyrrolidin-3-ol hydrochloride (PharmaBlock, 88 mg, 0.64 mmol) were added to a solution of 2-(8-chloropyrido[2,3-d]pyridazin-5-yl)-5-(trifluoromethyl)phenol (148 mg, 0.45 mmol) in NMP (1.5 ml). The mixture was heated to 80° C. and stirred for 2 h. The reaction was then cooled to room temperature and purified directly via preparative reverse phase HPLC (C18 stationary phase, MeCN/H₂O+0.05% FA) to afford the title compound. ¹H NMR (500 MHz, DMSO-d₆) δ 10.51 (s, 1H), 9.09 (dd, J=4.2, 1.5 Hz, 1H), 7.85 (dd, J=8.4, 1.5 Hz, 1H), 7.78 (dd, J=8.4, 4.2 Hz, 1H), 7.58 (d, J=7.8 Hz, 1H), 7.32 (d, J=8.0 Hz, 1H), 7.29 (s, 1H), 4.82 (s, 1H), 4.11 (s, 3H), 3.95 (s, 1H), 2.02-1.96 (m, 1H), 1.96-1.87 (m, 1H), 1.42 (s, 3H). LCMS [M+H]⁺=391.2 (calcd. 391.1).

TABLE 4
The following compounds were prepared using a procedure similar to that described for
Example 22 using the appropriate starting materials.

| | | | LCMS |
|---|---|---|---|
| Example | Structure | Name | [M + H]⁺ |
| 23 | 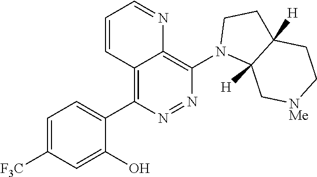 [SMILES: CN1CC[C@H]2CCN(c3nnc(-c4ccc(C(F)(F)F)cc4O)c4cccnc34)[C@H]2C1] | 2-(8-((3aS,7aR)-6- methyloctahydro-1H- pyrrolo[2,3-c]pyridin-1- yl)pyrido[2,3-d]pyridazin-5- yl)-5-(trifluoromethyl)phenol | Calcd. 430.2, found 430.2 |
| 24 | 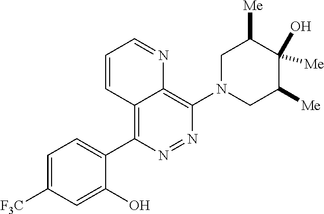 [SMILES: C[C@@H]1CN(c2nnc(-c3ccc(C(F)(F)F)cc3O)c3cccnc23)C[C@H](C)[C@]1(C)O] | (3S,4s,5R)-1-(5-(2-hydroxy-4- (trifluoromethyl)phenyl)pyrido [2,3-d]pyridazin-8-yl)-3,4,5- trimethylpiperidin-4-ol | Calcd. 433.2, found 433.2 |
| 25 | 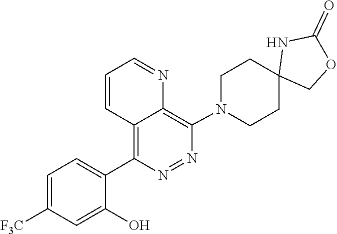 [SMILES: O=C1NC2(CCN(c3nnc(-c4ccc(C(F)(F)F)cc4O)c4cccnc34)CC2)CO1] | 8-(5-(2-hydroxy-4- (trifluoromethyl)phenyl)pyrido [2,3-d]pyridazin-8-yl)-3-oxa- 1,8-diazaspiro[4.5]decan-2-one | Calcd. 446.1, found 446.6 |
| 26 | 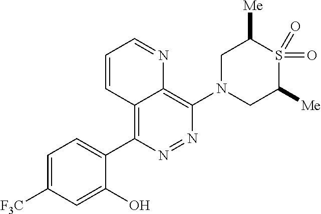 [SMILES: C[C@@H]1CN(c2nnc(-c3ccc(C(F)(F)F)cc3O)c3cccnc23)C[C@H](C)S1(=O)=O] | (cis)-4-(5-(2-hydroxy-4- (trifluoromethyl)phenyl)pyrido [2,3-d]pyridazin-8-yl)-2,6- dimethylthiomorpholine 1,1- dioxide | Calcd. 453.1, found 453.1 |
| 27 | 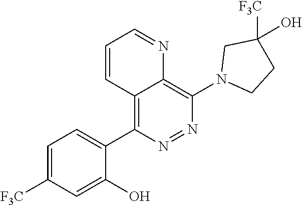 [SMILES: Oc1cc(C(F)(F)F)ccc1-c1nnc(N2CCC(O)(C(F)(F)F)C2)c2ncccc12] | (R and S)-1-(5-(2-hydroxy-4- (trifluoromethyl)phenyl)pyrido [2,3-d]pyridazin-8-yl)-3- (trifluoromethyl)pyrrolidin-3- ol | Calcd. 445.1, found 445.2 |
| 28 | 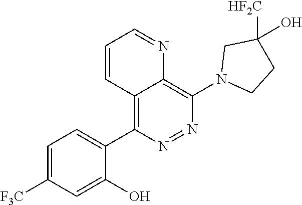 | (R and S)-3-(difluoromethyl)- 1-(5-(2-hydroxy-4- (trifluoromethyl)phenyl)pyrido [2,3-d]pyridazin-8- yl)pyrrolidin-3-ol | Calcd. 427.1, found 427.1 |
| 29 | 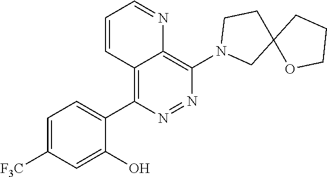 [SMILES: Oc1cc(C(F)(F)F)ccc1-c1nnc(N2CCC3(CCCO3)C2)c2ncccc12] | (R and S)-2-(8-(1-oxa-7- azaspiro[4.4]nonan-7- yl)pyrido[2,3-d]pyridazin-5- yl)-5-(trifluoromethyl)phenol | Calcd. 417.2, found 417.2 |
| 30 | 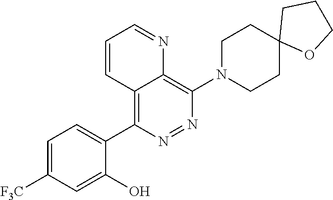 [SMILES: Oc1cc(C(F)(F)F)ccc1-c1nnc(N2CCC3(CCCO3)CC2)c2ncccc12] | 2-(8-(1-oxa-8- azaspiro[4.5]decan-8- yl)pyrido[2,3-d]pyridazin-5- yl)-5-(trifluoromethyl)phenol | Calcd. 431.2, found 431.3 |
| 31 | 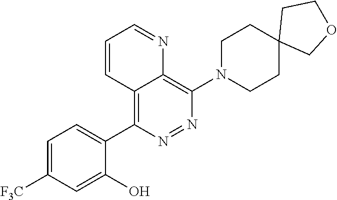 [SMILES: Oc1cc(C(F)(F)F)ccc1-c1nnc(N2CCC3(CCOC3)CC2)c2ncccc12] | 2-(8-(2-oxa-8- azaspiro[4.5]decan-8- yl)pyrido[2,3-d]pyridazin-5- yl)-5-(trifluoromethyl)phenol | Calcd. 431.2, found 431.3 |
| 32 | 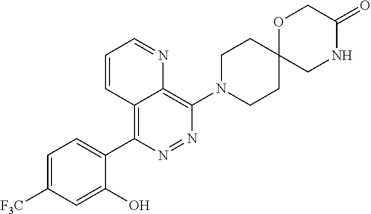 [SMILES: O=C1COC2(CCN(c3nnc(-c4ccc(C(F)(F)F)cc4O)c4cccnc34)CC2)CN1] | 9-(5-(2-hydroxy-4- (trifluoromethyl)phenyl)pyrido [2,3-d]pyridazin-8-yl)-1-oxa- 4,9-diazaspiro[5.5]undecan-3- one | Calcd. 460.2, found 460.3 |
| 33 | 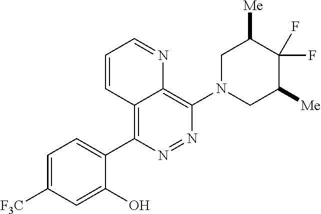 [SMILES: C[C@@H]1CN(c2nnc(-c3ccc(C(F)(F)F)cc3O)c3cccnc23)C[C@H](C)C1(F)F] | 2-(8-((cis)-4,4-difluoro-3,5- dimethylpiperidin-1- yl)pyrido[2,3-d]pyridazin-5- yl)-5-(trifluoromethyl)phenol | Calcd. 439.2, found 439.1 |
| 34 | 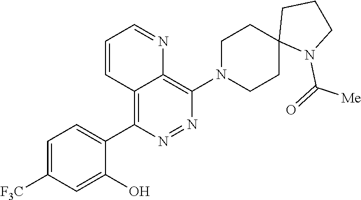 [SMILES: CC(=O)N1CCCC12CCN(c1nnc(-c3ccc(C(F)(F)F)cc3O)c3cccnc13)CC2] | 1-(8-(5-(2-hydroxy-4- (trifluoromethyl)phenyl)pyrido [2,3-d]pyridazin-8-yl)-1,8- diazaspiro[4.5]decan-1- yl)ethan-1-one | Calcd. 472.2, found 472.1 |
| 35 | 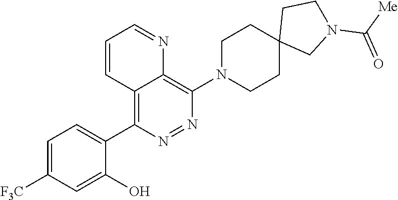 [SMILES: CC(=O)N1CCC2(CCN(c3nnc(-c4ccc(C(F)(F)F)cc4O)c4cccnc34)CC2)C1] | 1-(8-(5-(2-hydroxy-4- (trifluoromethyl)phenyl)pyrido [2,3-d]pyridazin-8-yl)-2,8- diazaspiro[4.5]decan-2- yl)ethan-1-one | Calcd. 472.2, found 472.1 |
| 36 | 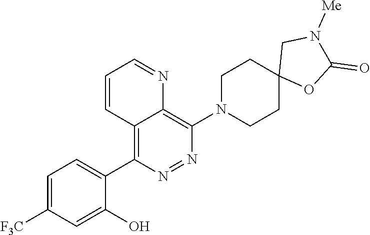 [SMILES: CN1CC2(CCN(c3nnc(-c4ccc(C(F)(F)F)cc4O)c4cccnc34)CC2)OC1=O] | 8-(5-(2-hydroxy-4-(trifluoro- methyl)phenyl)pyrido[2,3-d]- pyridazin-8-yl)-3-methyl-1- oxa-3,8-diazaspiro[4.5]decan- 2-one | Calcd. 460.2, found 460.1 |
| 37 | 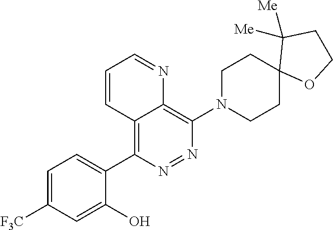 [SMILES: CC1(C)CCOC12CCN(c1nnc(-c3ccc(C(F)(F)F)cc3O)c3cccnc13)CC2] | 2-(8-(4,4-dimethyl-1-oxa-8- azaspiro[4.5]decan-8- yl)pyrido[2,3-d]pyridazin-5- yl)-5-(trifluoromethyl)phenol | Calcd. 459.2, found 459.1 |
| 38 | 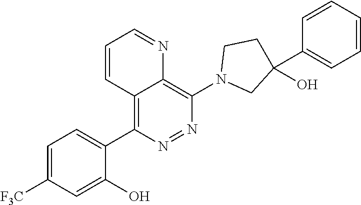 [SMILES: Oc1cc(C(F)(F)F)ccc1-c1nnc(N2CCC(O)(c3ccccc3)C2)c2ncccc12] | (R and S)-1-(5-(2-hydroxy-4- (trifluoromethyl)phenyl)pyrido [2,3-d]pyridazin-8-yl)-3- phenylpyrrolidin-3-ol | Calcd. 453.2, found 453.1 |
| 39 | 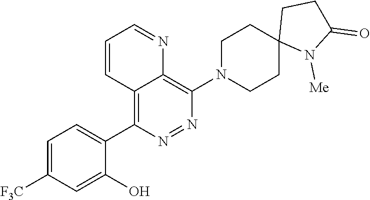 [SMILES: CN1C(=O)CCC12CCN(c1nnc(-c3ccc(C(F)(F)F)cc3O)c3cccnc13)CC2] | 8-(5-(2-hydroxy-4- (trifluoromethyl)phenyl)pyrido [2,3-d]pyridazin-8-yl)-1- methyl-1,8- diazaspiro[4.5]decan-2-one | Calcd. 458.2, found 458.1 |
| 40 | 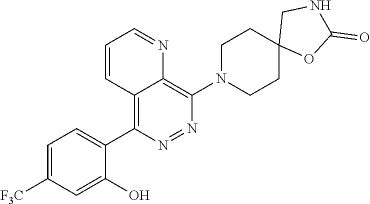 [SMILES: O=C1NCC2(CCN(c3nnc(-c4ccc(C(F)(F)F)cc4O)c4cccnc34)CC2)O1] | 8-(5-(2-hydroxy-4- (trifluoromethyl)phenyl)pyrido [2,3-d]pyridazin-8-yl)-1-oxa- 3,8-diazaspiro[4.5]decan-2-one | Calcd. 446.1, found 446.0 |
| 41 | 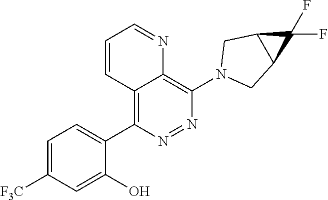 [SMILES: Oc1cc(C(F)(F)F)ccc1-c1nnc(N2C[C@@H]3[C@H](C2)C3(F)F)c2ncccc12] | 2-(8-(6,6-difluoro-3- azabicyclo[3.1.0]hexan-3- yl)pyrido[2,3-d]pyridazin-5- yl)-5-(trifluoromethyl)phenol | Calcd. 409.1, found 409.1 |
| 42 | 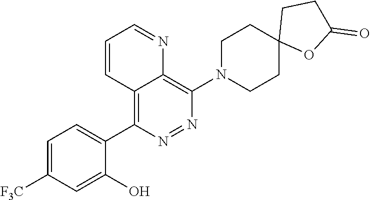 [SMILES: O=C1CCC2(CCN(c3nnc(-c4ccc(C(F)(F)F)cc4O)c4cccnc34)CC2)O1] | 8-(5-(2-hydroxy-4- (trifluoromethyl)phenyl)pyrido [2,3-d]pyridazin-8-yl)-1-oxa-8- azaspiro[4.5]decan-2-one | Calcd. 445.2, found 445.3 |
| 43 | 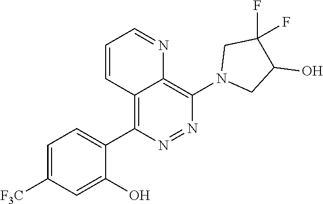 [SMILES: Oc1cc(C(F)(F)F)ccc1-c1nnc(N2CC(O)C(F)(F)C2)c2ncccc12] | (R and S)-4,4-difluoro-1-(5-(2- hydroxy-4- (trifluoromethyl)phenyl)pyrido [2,3-d]pyridazin-8- yl)pyrrolidin-3-ol | Calcd. 413.1, found 413.3 |
| 44 | 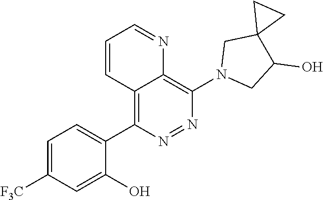 [SMILES: Oc1cc(C(F)(F)F)ccc1-c1nnc(N2CC(O)C3(CC3)C2)c2ncccc12] | (R and S)-5-(5-(2-hydroxy-4- (trifluoromethyl)phenyl)pyrido [2,3-d]pyridazin-8-yl)-5- azaspiro[2.4]heptan-7-ol | Calcd. 403.1, found 403.2 |
| 45 | 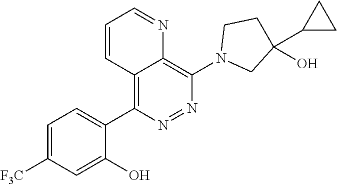 [SMILES: Oc1cc(C(F)(F)F)ccc1-c1nnc(N2CCC(O)(C3CC3)C2)c2ncccc12] | (R and S)-3-cyclopropyl-1-(5- (2-hydroxy-4- (trifluoromethyl)phenyl)pyrido [2,3-d]pyridazin-8- yl)pyrrolidin-3-ol | Calcd. 417.2, found 417.2 |
| 46 | 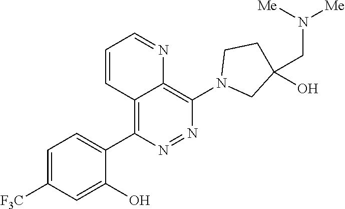 [SMILES: CN(C)CC1(O)CCN(c2nnc(-c3ccc(C(F)(F)F)cc3O)c3cccnc23)C1] | (R and S)-3- ((dimethylamino)methyl)-1-(5- (2-hydroxy-4- (trifluoromethyl)phenyl)pyrido [2,3-d]pyridazin-8- yl)pyrrolidin-3-ol | Calcd. 434.2, found 434.3 |
| 47 | 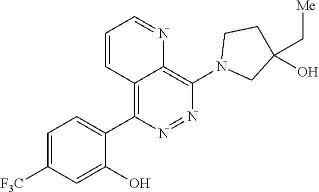 [SMILES: CCC1(O)CCN(c2nnc(-c3ccc(C(F)(F)F)cc3O)c3cccnc23)C1] | (R and S)-3-ethyl-1-(5-(2- hydroxy-4- (trifluoromethyl)phenyl)pyrido [2,3-d]pyridazin-8- yl)pyrrolidin-3-ol | Calcd. 405.2, found 405.0 |
| 48 | 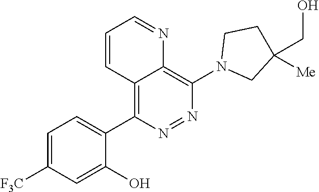 [SMILES: CC1(CO)CCN(c2nnc(-c3ccc(C(F)(F)F)cc3O)c3cccnc23)C1] | (R and S)-2-(8-(3- (hydroxymethyl)-3- methylpyrrolidin-1- yl)pyrido[2,3-d]pyridazin-5- yl)-5-(trifluoromethyl)phenol | Calcd. 405.2, found 405.0 |
| 49 | 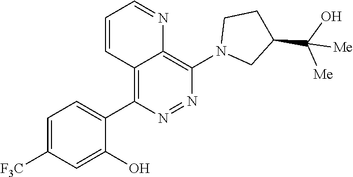 [SMILES: CC(C)(O)[C@@H]1CCN(c2nnc(-c3ccc(C(F)(F)F)cc3O)c3cccnc23)C1] | (R)-2-(8-(3-(2-hydroxypropan- 2-yl)pyrrolidin-1- yl)pyrido[2,3-d]pyridazin-5- yl)-5-(trifluoromethyl)phenol | Calcd. 419.2, found 419.1 |
| 50 | 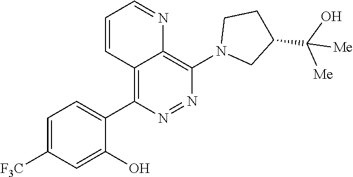 [SMILES: CC(C)(O)[C@H]1CCN(c2nnc(-c3ccc(C(F)(F)F)cc3O)c3cccnc23)C1] | (S)-2-(8-(3-(2-hydroxypropan- 2-yl)pyrrolidin-1- yl)pyrido[2,3-d]pyridazin-5- yl)-5-(trifluoromethyl)phenol | Calcd. 419.2, found 419.1 |

TABLE 5
The following compounds were prepared using procedures similar to those described for
Example 22 using the appropriate starting materials. Enantiomeric products were separated using
chiral SFC methods specified in the table. For pairs of enantiomers, the fast-eluting isomer is
listed first.

| | | | LCMS | Chiral |
|---|---|---|---|---|
| Example | Structure | Name | [M + H]⁺ | Method |
| 51 | 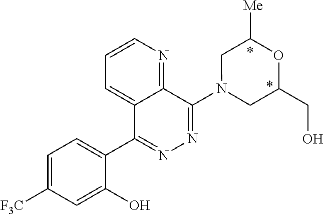 [SMILES: CC1CN(c2nnc(-c3ccc(C(F)(F)F)cc3O)c3cccnc23)CC(CO)O1] | 2-(8-((2R,6R or 2S,6S)-2- (hydroxymethyl)-6- methylmorpholino) pyrido[2,3- d]pyridazin-5-yl)-5- (trifluoromethyl) phenol | Calcd. 421.2, found 421.2 | F |
| 52 | 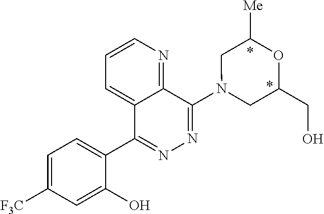 [SMILES: CC1CN(c2nnc(-c3ccc(C(F)(F)F)cc3O)c3cccnc23)CC(CO)O1] | 2-(8-((2S,6S or 2R,6R)-2-(hydroxy- methyl)-6-methyl- morpholino)pyrido[2, 3-d]pyridazin-5-yl)- 5-(trifluoro- methyl)phenol | Calcd. 421.2, found 421.2 | F |

### Example 53

### 1-((2R,4r,6S)-2,6-dimethyltetrahydro-2H-pyran-4-yl)-4-(2-methyl-4-(trifluoromethyl)phenyl)phthalazine

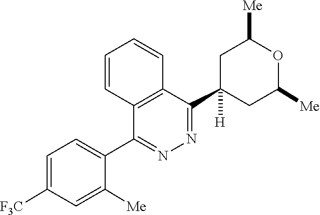 [SMILES: Cc1cc(C(F)(F)F)ccc1-c1nnc([C@H]2C[C@@H](C)O[C@@H](C)C2)c2ccccc12]

Step 1: 1-chloro-4-(2-methyl-4-(trifluoromethyl)phenyl)phthalazine: To a solution of (2-methyl-4-(trifluoromethyl)phenyl)boronic acid (Combi-Blocks, 510 mg, 2.5 mmol) in 1,4-dioxane (16.7 mL) and H₂O (8.3 mL) were added 1,4-dichlorophthalazine (Combi-Blocks, 498 mg, 2.5 mmol), K₂CO₃ (1.38 g, 10.0 mmol) and PdCl₂(dppf) (183 mg, 0.25 mmol). The mixture was degassed with N₂ and then stirred at 100° C. for 2 h. After cooling to room temperature, the mixture was quenched with saturated aqueous NaHCO₃ and partitioned between H₂O and EtOAc. The layers were separated and the aqueous phase was extracted with EtOAc. The combined organic layers were dried over MgSO₄, filtered and concentrated under vacuum. The resulting crude residue was purified by silica gel chromatography (EtOAc:hexanes), followed by reverse phase HPLC (C18 stationary phase, MeCN/H₂O+0.1% TFA). The combined product fractions were quenched with saturated aqueous NaHCO₃ and diluted with EtOAc. The layers were separated, and the aqueous phase was extracted with EtOAc (×3). The combined organic layers were dried over MgSO₄, filtered and concentrated under reduced pressure. Additional purification by silica gel chromatography (EtOAc:hexanes) afforded the title compound. LCMS [M+H]⁺=323.1 (calcd. 323.1).

Step 2: 1-((2R,4r,6S)-2,6-dimethyltetrahydro-2H-pyran-4-yl)-4-(2-methyl-4-(trifluoro-methyl)phenyl)phthalazine: A catalyst solution consisting of dtbbpy (14 mg, 0.05 mmol) and Ni(DME)Br₂ (16 mg, 0.05 mmol) in DMA (0.7 mL) was degassed with Ar for 10 min. In a separate vial cis-1,3-dioxoisoindolin-2-yl 2,6-dimethyltetrahydro-2H-pyran-4-carboxylate (78 mg, 0.26 mmol), 1-chloro-4-(2-methyl-4-(trifluoromethyl)phenyl)-phthalazine (55 mg, 0.17 mmol), and Zn powder (Strem, 22 mg, 0.34 mmol) were placed under Ar, and the catalyst solution (0.7 mL) was added. The vial was sealed and heated to 50° C. with vigorous stirring (>1000 rpm) overnight. The reaction mixture was cooled to room temperature, filtered and purified directly via preparative reverse phase HPLC (C18 stationary phase, MeCN/H₂O+0.05% FA) to afford the title compound. ¹H NMR (500 MHz, CD₃CN) δ 8.40 (d, J=8.4 Hz, 1H), 7.99 (ddd, J=8.3, 7.1, 1.2 Hz, 1H), 7.86 (td, J=7.6, 7.1, 1.0 Hz, 1H), 7.77 (s, 1H), 7.70 (d, J=7.9 Hz, 1H), 7.53 (dd, J=8.0, 4.5 Hz, 2H), 4.00 (tt, J=12.0, 3.6 Hz, 1H), 3.82 (dqd, J=12.4, 6.2, 1.8 Hz, 2H), 2.11 (s, 3H), 2.02 (d, J=11.5 Hz, 2H), 1.83-1.71 (m, 2H), 1.24 (d, J=6.2 Hz, 6H). LCMS [M+H]⁺=401.2 (calcd. 401.2).

### Examples 54 and 55

### (2R,4s,6S)-4-(5-(2-hydroxy-4-(trifluoromethyl)phenyl)pyrido[2,3-d]-pyridazin-8-yl)-2,6-dimethyltetrahydro-2H-pyran-4-ol (Example 54) and (2R,4r,6S)-4-(5-(2-hydroxy-4-(trifluoromethyl)phenyl)pyrido[2,3-d]-pyridazin-8-yl)-2,6-dimethyltetrahydro-2H-pyran-4-ol (Example 55)

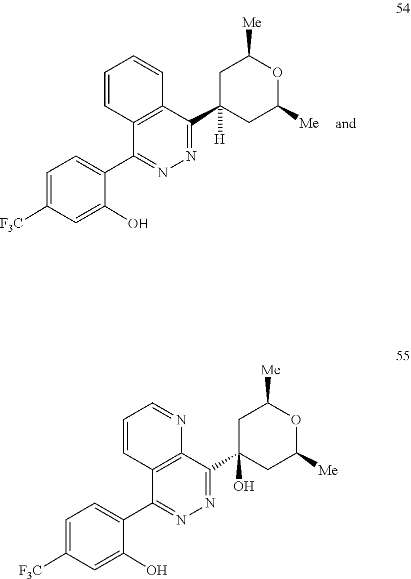 [SMILES: C[C@@H]1C[C@H](c2nnc(-c3ccc(C(F)(F)F)cc3O)c3ccccc23)C[C@H](C)O1 ; "and" ; C[C@@H]1C[C@@](O)(c2nnc(-c3ccc(C(F)(F)F)cc3O)c3cccnc23)C[C@H](C)O1]

Step 1: 2-(8-((2R,6S and 2S,6R)-2,6-dimethyl-3,6-dihydro-2H-pyran-4-yl)pyrido[2,3-d]pyridazin-5-yl)-5-(trifluoromethyl)phenol: 2-(8-chloropyrido[2,3-d]pyridazin-5-yl)-5-(trifluoromethyl)phenol (300 mg, 0.92 mmol) and XPhos Pd G3 (78 mg, 0.09 mmol) were added to a solution of 2-((2R,6S and 2S,6R)-2,6-dimethyl-3,6-dihydro-2H-pyran-4-yl)-4,4,5,5-tetramethyl-1,3,2-dioxaborolane (J&W Pharmlab, 373 mg, 1.57 mmol) in 1,4-dioxane (7.4 mL). The reaction mixture was degassed for 10 min by sparging with Ar, followed by the addition of 1 M aq. K₃PO₄ (2.0 mL, 2.03 mmol). The reaction mixture was heated to 100° C. and stirred for 2 h. Then the reaction mixture was cooled to room temperature, diluted with EtOAc, dry loaded onto silica gel, and purified by silica gel chromatography (EtOAc:hexanes) to afford the title compound. LCMS [M+H]⁺=402.1 (calcd. 401.1).

Step 2: (2R,4s,6S)-4-(5-(2-hydroxy-4-(trifluoromethyl)phenyl)pyrido[2,3-d]pyridazin-8-yl)-2,6-dimethyltetrahydro-2H-pyran-4-ol and (2R,4r,6S)-4-(5-(2-hydroxy-4-(trifluoromethyl)-phenyl)pyrido[2,3-d]pyridazin-8-yl)-2,6-dimethyltetrahydro-2H-pyran-4-ol: 2-(8-((2R,6S and 2S,6R)-2,6-dimethyl-3,6-dihydro-2H-pyran-4-yl)pyrido[2,3-d]pyridazin-5-yl)-5-(trifluoromethyl)phenol (54 mg, 0.135 mmol) and Mn(TMHD)₃ (98 mg, 0.16 mmol) were suspended in ⁱPrOH (1.4 mL). The mixture was sparged with O₂ for 5 min, followed by addition of PhSiH₃ (33 μL, 0.27 mmol). The mixture was sealed under an O₂ atmosphere, heated to 60° C., and stirred for 2 h. Then the reaction mixture was cooled to room temperature, quenched with excess P(OMe)₃ and stirred for 5 min. The mixture was diluted with H₂O, followed by 1M aq. HCl, and EtOAc. The layers were separated, and the aqueous layer was neutralized to pH 7 and back extracted with EtOAc. The combined organic layers were dried with Na₂SO₄, filtered, and concentrated under reduced pressure. The resulting residue was purified via silica gel chromatography (EtOAc:hexanes) to afford the title compounds as two separated diastereomers. The faster eluting diastereomer was obtained (Example 54). ¹H NMR (500 MHz, CD₃CN) δ 9.23 (dd, J=4.2, 1.4 Hz, 1H), 8.38 (dd, J=8.6, 1.4 Hz, 1H), 7.91 (dd, J=8.5, 4.3 Hz, 1H), 7.71 (d, J=7.7 Hz, 1H), 7.43-7.34 (m, 2H), 6.59 (s, 1H), 4.17 (dq, J=11.0, 6.3 Hz, 2H), 2.08 (d, J=13.0 Hz, 2H), 1.82-1.79 (m, 2H), 1.21 (d, J=6.2 Hz, 6H). LCMS [M+H]⁺=420.1 (calcd. 420.2). The slower eluting isomer of the title compound was obtained (Example 55). ¹H NMR (500 MHz, CD₃CN) δ 9.25-9.20 (m, 1H), 8.40 (dd, J=8.6, 1.1 Hz, 1H), 7.91 (dd, J=8.5, 4.3 Hz, 1H), 7.72 (d, J=7.8 Hz, 1H), 7.42-7.39 (m, 2H), 6.36 (s, 1H), 3.86 (dq, J=11.4, 6.3 Hz, 2H), 2.75 (d, J=13.1 Hz, 2H), 1.71-1.62 (m, 2H), 1.14 (d, J=6.2 Hz, 6H). LCMS [M+H]⁺=420.1 (calcd. 420.2).

### Examples 56 and 57

### (S)-1-(2-(difluoromethyl)-5-(2-hydroxy-4-(trifluoromethyl)phenyl)pyrido[2,3-d]pyridazin-8-yl-3-methylpyrrolidin-3-ol (Example 56); and (S)-1-(3-(difluoromethyl)-5-(2-hydroxy-4-(trifluoromethyl)phenyl)pyrido[2,3-d]pyridazin-8-yl-3-methylpyrrolidin-3-ol (Example 57)

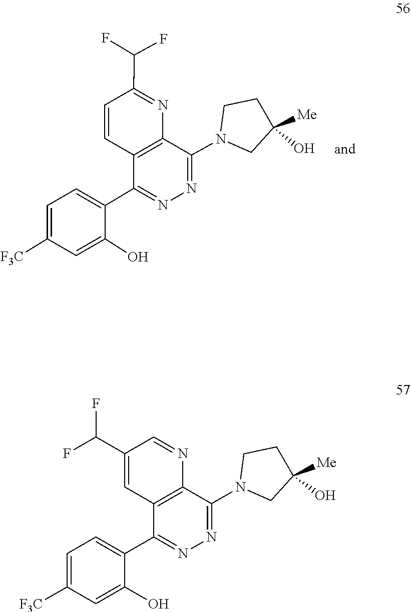 [SMILES: C[C@]1(O)CCN(c2nnc(-c3ccc(C(F)(F)F)cc3O)c3ccc(C(F)F)nc23)C1 ; "and" ; C[C@]1(O)CCN(c2nnc(-c3ccc(C(F)(F)F)cc3O)c3cc(C(F)F)cnc23)C1]

(S)-1-(5-(2-hydroxy-4-(trifluoromethyl)phenyl)pyrido[2,3-d]pyridazin-8-yl)-3-methylpyrrolidin-3-ol (Example 22, 60 mg, 0.15 mmol) and DFMS (136 mg, 0.46 mmol) was suspended in a mixture of CHCl₃ (0.53 mL) and H₂O (0.23 mL). Then TBHP (70 wt. % in H₂O, 85 μL, 0.62 mmol) was added dropwise with stirring, and the mixture was heated to 50° C. for 3 h. Additional DFMS (91 mg, 0.307 mmol) and TBHP (70 wt. % in H₂O, 50 μL, 0.38 mmol) were added, and the mixture was allowed to stir at 50° C. overnight. The reaction mixture was cooled to room temperature, and the solvents removed under reduced pressure. The resulting crude residue was purified by reverse phase HPLC (C18 stationary phase, MeCN/H₂O+0.05% FA) to give the title compounds as a mixture of regioisomers, which were subsequently resolved by chiral SFC (Method G). The faster eluting isomer was obtained (Example 56). ¹H NMR (500 MHz, CD₃CN) δ 8.35 (d, J=8.6 Hz, 1H), 7.94 (d, J=8.7 Hz, 1H), 7.63 (d, J=7.9 Hz, 1H), 7.25-7.19 (m, 2H), 6.91 (t, J=54.9 Hz, 1H), 4.33-4.08 (m, 3H), 3.95 (s, 1H), 2.06-1.97 (m, 2H), 1.48 (s, 3H). LCMS [M+H]⁺=441.0 (calcd. 441.1). The slower eluting diastereomer was obtained (Example 57). ¹H NMR (500 MHz, CD₃CN) δ 9.18 (s, 1H), 8.31 (s, 1H), 7.63 (d, J=7.8 Hz, 1H), 7.22 (d, J=8.0 Hz, 2H), 7.00 (t, J=55.0 Hz, 1H), 4.28-4.09 (m, 3H), 3.96 (d, J=11.1 Hz, 1H), 2.05-1.99 (m, 2H), 1.48 (s, 3H). LCMS [M+H]⁺=441.0 (calcd. 441.1).

Example of a Pharmaceutical Composition

As a specific embodiment of an oral pharmaceutical composition, a 100 mg potency tablet is composed of 100 mg of any one of the Examples, 268 mg microcrystalline cellulose, 20 mg of croscarmellose sodium, and 4 mg of magnesium stearate. The active, microcrystalline cellulose, and croscarmellose are blended first. The mixture is then lubricated by magnesium stearate and pressed into tablets.

Biological Assay

Activation of the canonical NLRP3 inflammasome requires two steps, priming and activation. A priming signal such as a pathogen activated molecular patterns (PAMPs) or danger-activated molecular patterns (DAMPs) are recognized by Toll-like receptors leads to nuclear factor kappa B (NF-KB)-mediated signaling. This in turn, up-regulates transcription of inflammasome-related components, including inactive NLRP3 and proIL-1β (Bauernfeind et al., J. Immunol. 2009, 183, 787-791: Franchi et al. Nat. Immunol. 2012, 13, 325-332; Franchi et al., J. Immunol. 2014, 193, 4214-4222). The second step is activation which induces oligomerization of NLRP3 and subsequent assembly of NLRP3, apoptosis-associated speck-like protein containing a CARD (ASC), and procaspase-1 into an inflammasome complex. This triggers the transformation of procaspase-1 to caspase-1, and the production and secretion of mature IL-1β and IL-18 (Kim et al., J. Inflamm. 2015, 12, 41; Ozaki et al. Inflamm. Res. 2015, 8, 15-27; Rabeony et al., Eur. J. Immunol. 2015, 45, 2847). During inflammasome complex assembly, the oligomerization of NLRP3 triggers the nucleation of ASC and an event commonly referred to as “ASC SPECK” formation as it is identified in the cell as a discrete puncta within the cell after staining and visualization of ASC using common immunocytochemical methods.

The ability of compounds to inhibit NLRP3 inflammasome activation was determined in vitro by monitoring formation of the ASC-SPECK in human monocytic THP-1 cells after stimulation. THP-1 cells (ATCC catalog #TIB-202) were maintained in complete growth media containing Roswell Park Memorial Institute RPMI (ATCC catalog #30-2001), 10% heat inactivated fetal bovine serum, 1× penicillin/streptomycin and 0.05 mM 2-mercaptoethanol. At the start of the assay, undifferentiated THP-1 cells were plated at a density of 20,000 cells per well in a 384-well plate (Poly-D-lysine coated Cell Carrier Ultra microplate, Perkin Elmer catalog #6057500) in complete growth media supplemented with 10 ng/ml phorbol 12-myristate 13-acetate (PMA; Sigma catalog #P8139), and then incubated overnight. The next day, media was replaced with assay media [RPMI (Gibco catalog #11875-093), 0.01% bovine serum albumin (BSA)]. Compounds were serially diluted in DMSO and then added to wells one hour prior to the addition of 12.5 ug/ml Gramicidin (Enzo Lifescience, catalog #ALX-350-233-M005). All incubations were carried out at 37° C. (5% CO₂/95% air). Following a 3-hour treatment with gramicidin, cells are fixed with 4% paraformaldehyde and stored at 4° C. until immunofluorescence staining.

Immunofluorescence staining: Anti-ASC antibody (MBL catalog #D086-3) was desalted and labeled with Alexa 488 antibody labeling kit (Thermo catalog #A20181) prior to use as described below. After fixation, the following steps were carried out at room temperature. Cells were first permeabilized with 0.3% Triton X-100 in phosphate-buffered saline (PBS) for 15 minutes and then incubated in blocking buffer containing 5% goat serum, 0.3% tween-20 and 0.03% sodium azide in PBS for 1 hour. Cells were stained with a mixture of ASC-Alexa 488 antibody (diluted 1:200 in blocking buffer) and nuclear stain DRAQ5 (1:5000 in blocking buffer, Thermo catalog #62251) in blocking buffer for 1 hour. Following a wash with 0.3% Tween-20 in PBS, plates were imaged with an Opera Phenix High Content Screening System. The number of DRAQ5 positive cells containing ASC SPECKS were quantified in each well.

Data analysis: EC₅₀ values were calculated by standard curve-fitting analysis using an internally developed program in TIBCO Spotfire software.

The compounds of the present invention inhibit NLRP3 inflammasome activation in the above Biological Assay and have EC₅₀ values of less than 5 micromolar. Specific EC₅₀ values of the compounds of Examples 1-57 in the above Biological Assay are listed in Table I.

TABLE I
EC₅₀ values (nM) for Examples that inhibit NLRP3 inflammasome
activation in the above Biological Assay

| Example | EC₅₀ (nM) |
|---|---|
| 1 | 356 |
| 2 | 49 |
| 3 | 891 |
| 4 | 82 |
| 5 | 556 |
| 6 | 1851 |
| 7 | 570 |
| 8 | 515 |
| 9 | 80 |
| 10 | 60 |
| 11 | 750 |
| 12 | 621 |
| 13 | 264 |
| 14 | 734 |
| 15 | 3679 |
| 16 | 70 |
| 17 | 201 |
| 18 | 23 |
| 19 | 68 |
| 20 | 33 |
| 21 | 815 |
| 22 | 124 |
| 23 | 5 |
| 24 | 52 |
| 25 | 148 |
| 26 | 96 |
| 27 | 1229 |
| 28 | 447 |
| 29 | 353 |
| 30 | 1270 |
| 31 | 915 |
| 32 | 330 |
| 33 | 221 |
| 34 | 1509 |
| 35 | 420 |
| 36 | 742 |
| 37 | 1131 |
| 38 | 1601 |
| 39 | 563 |
| 40 | 304 |
| 41 | 999 |
| 42 | 952 |
| 43 | 482 |
| 44 | 591 |
| 45 | 1184 |
| 46 | 831 |
| 47 | 494 |
| 48 | 411 |
| 49 | 1790 |
| 50 | 1331 |
| 51 | 65 |
| 52 | 74 |
| 53 | 3966 |
| 54 | 1103 |
| 55 | 1341 |
| 56 | 90 |
| 57 | 1568 |

The scope of the claims should not be limited by the preferred embodiments set forth in the examples, but should be given the broadest interpretation consistent with the description as a whole.

While the invention has been described and illustrated with reference to certain particular embodiments thereof, those skilled in the art will appreciate that various adaptations, changes, modifications, substitutions, deletions, or additions of procedures and protocols may be made without departing from the scope of the invention. For example, effective dosages other than the particular dosages as set forth herein above may be applicable as a consequence of variations in responsiveness of the mammal being treated for any of the indications with the compounds of the invention indicated above. The specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention.

1. A compound of structural Formula I: 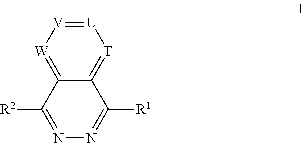
or a pharmaceutically acceptable salt thereof, wherein
T is independently selected from the group:
1) CR³, and
2) N,
provided that zero, one or two of T, U, V and W are N;
U is independently selected from the group:
1) CR⁴, and
2) N;
V is independently selected from the group:
1) CR⁵, and
2) N;
W is independently selected from the group:
1) CR⁶, and
2) N;
R¹ is selected from the group:
1) —C₃₋₁₂cycloalkyl,
2) —C₃₋₁₂cycloalkenyl, and
3) —C₂₋₁₂cycloheteroalkyl,
wherein R¹ is unsubstituted or substituted with one to six substituents selected from R^{a};
R² is selected from the group:
1) aryl, and
2) heteroaryl,
wherein R² is unsubstituted or substituted with one to five substituents selected from R^{b};
R³ is selected from the group:
1) hydrogen,
2) OH,
3) CN,
4) CF₃,
5) CF₂H,
6) —C₁₋₆alkyl,
7) —O—C₁₋₆alkyl,
8) halogen,
9) —C₃₋₆cycloalkyl,
10) —C₂₋₆cycloheteroalkyl,
11) —C₁₋₆alkyl-O—C₁₋₆alkyl,
12) —(CH₂)ᵣC(O)R^{h},
13) —(CH₂)ᵣC(O)N(Rⁱ)₂,
14) —(CH₂)ᵣN(Rⁱ)C(O)R^{h},
15) —(CH₂)ᵣN(Rⁱ)C(O)OR^{h},
16) —(CH₂)ᵣN(Rⁱ)C(O)N(Rⁱ)₂,
17) —(CH₂)ᵣN(R^{j})C(O)N(Rⁱ)₂,
18) —(CH₂)ᵣN(Rⁱ)S(O)ₘR^{h},
19) —(CH₂)ᵣN(Rⁱ)S(O)ₘN(Rⁱ)₂,
20) —(CH₂)ᵣN(Rⁱ)S(O)ₘN(Rⁱ)₂, and
21) —(CH₂)ᵣN(Rⁱ)₂,
wherein R³ is unsubstituted or substituted with one to five substituents selected from R^{d};
R⁴ is selected from the group:
1) hydrogen,
2) OH,
3) CN,
4) CF₃,
5) CF₂H,
6) —C₁₋₆alkyl,
7) —O—C₁₋₆alkyl,
8) halogen,
9) —C₃₋₆cycloalkyl,
10) —C₂₋₆cycloheteroalkyl,
11) —C₁₋₆alkyl-O—C₁₋₆alkyl,
12) —(CH₂)ₛC(O)R^{h},
13) —(CH₂)ₛC(O)N(Rⁱ)₂,
14) —(CH₂)ₛN(Rⁱ)C(O)R^{h},
15) —(CH₂)ₛN(Rⁱ)C(O)OR^{h},
16) —(CH₂)ₛN(Rⁱ)C(O)N(Rⁱ)₂,
17) —(CH₂)ₛN(R^{j})C(O)N(Rⁱ)₂,
18) —(CH₂)ₛN(Rⁱ)S(O)ₘR^{h},
19) —(CH₂)ₛN(Rⁱ)S(O)ₘN(Rⁱ)₂,
20) —(CH₂)ₛN(Rⁱ)S(O)ₘN(Rⁱ)₂, and
21) —(CH₂)ₛN(Rⁱ)₂,
wherein R⁴ is unsubstituted or substituted with one to five substituents selected from R^{e};
R⁵ is selected from the group:
1) hydrogen,
2) OH,
3) CN,
4) CF₃,
5) CF₂H,
6) —C₁₋₆alkyl,
7) —O—C₁₋₆alkyl,
8) halogen,
9) —C₃₋₆cycloalkyl,
10) —C₂₋₆cycloheteroalkyl,
11) —C₁₋₆alkyl-O—C₁₋₆alkyl,
12) —(CH₂)ₜC(O)R^{h},
13) —(CH₂)ₜC(O)N(Rⁱ)₂,
14) —(CH₂)ₜN(R^{j})C(O)R^{h},
15) —(CH₂)ₜN(Rⁱ)C(O)OR^{h},
16) —(CH₂)ₜN(Rⁱ)C(O)N(Rⁱ)₂,
17) —(CH₂)ₜN(R^{j})C(O)N(Rⁱ)₂,
18) —(CH₂)ₜN(Rⁱ)S(O)ₘR^{h},
19) —(CH₂)ₜN(Rⁱ)S(O)ₘN(Rⁱ)₂,
20) —(CH₂)ₜN(Rⁱ)S(O)ₘN(Rⁱ)₂, and
21) —(CH₂)ₜN(Rⁱ)₂,
wherein R⁵ is unsubstituted or substituted with one to five substituents selected from R^{f};
R⁶ is selected from the group:
1) hydrogen,
2) OH,
3) CN,
4) CF₃,
5) CF₂H,
6) —C₁₋₆alkyl,
7) —O—C₁₋₆alkyl,
8) halogen,
9) —C₃₋₆cycloalkyl,
10) —C₂₋₆cycloheteroalkyl,
11) —C₁₋₆alkyl-O—C₁₋₆alkyl,
12) —(CH₂)ᵤC(O)R^{h},
13) —(CH₂)ᵤC(O)N(Rⁱ)₂,
14) —(CH₂)ᵤN(R^{j})C(O)R^{h},
15) —(CH₂)ᵤN(R^{j})C(O)OR^{h},
16) —(CH₂)ᵤN(R^{j})C(O)N(Rⁱ)₂,
17) —(CH₂)ᵤN(R^{j})C(O)N(Rⁱ)₂,
18) —(CH₂)ᵤN(R^{j})S(O)ₘR^{h},
19) —(CH₂)ᵤN(R^{j})S(O)ₘN(Rⁱ)₂,
20) —(CH₂)ᵤN(R^{j})S(O)ₘN(Rⁱ)₂, and
21) —(CH₂)ᵤN(Rⁱ)₂,
wherein R⁶ is unsubstituted or substituted with one to five substituents selected from R^{g};
each R^{a} is independently selected from the group:
1) CN,
2) oxo,
3) —OH,
4) halogen,
5) —C₁₋₆alkyl,
6) —O—C₁₋₆alkyl,
7) —C₂₋₆alkenyl,
8) —C₂₋₆alkynyl,
9) —C₃₋₆cycloalkyl,
10) —C₂₋₆cycloheteroalkyl,
11) aryl,
12) heteroaryl,
13) —C(O)C₁₋₆alkyl,
14) —C₁₋₆alkyl-aryl,
15) —C₁₋₆alkyl-heteroaryl,
16) —C₁₋₆alkyl-C₃₋₆cycloalkyl,
17) —C₁₋₆alkyl-C₂₋₆cycloheteroalkyl,
18) —C₂₋₆alkenyl-C₃₋₆cycloalkyl,
19) —C₂₋₆alkenyl-C₂₋₆cycloheteroalkyl,
20) —C₂₋₆alkenyl-aryl,
21) —C₂₋₆alkenyl-heteroaryl,
22) —C₂₋₆alkynyl-C₃₋₆cycloalkyl,
23) —C₂₋₆alkynylC₂₋₆cycloheteroalkyl,
24) —C₂₋₆alkynyl-aryl,
25) —C₂₋₆alkynyl-heteroaryl,
26) —(CH₂)ₚ—O—C₁₋₆alkyl,
27) —(CH₂)ₚ—O—C₂₋₆alkenyl,
28) —(CH₂)ₚ—O—C₂₋₆alkynyl,
29) —(CH₂)ₚ—O—C₃₋₆cycloalkyl,
30) —(CH₂)ₚ—O—C₂₋₆cycloheteroalkyl,
31) —(CH₂)ₚ—O-aryl,
32) —(CH₂)ₚ—O-heteroaryl,
33) —OC₁₋₆alkyl-C₃₋₆cycloalkyl,
34) —OC₁₋₆alkyl-C₂₋₆cycloheteroalkyl,
35) —OC₁₋₆alkyl-aryl,
36) —OC₁₋₆alkyl-heteroaryl,
37) —(CH₂)ₚ—S(O)ᵣR^{k},
38) —(CH₂)ₚ—S(O)N(R^{L})₂, and
39) —(CH₂)ₚ—N(R^{L})₂,
wherein each R^{a} is unsubstituted or substituted with one to six substituents selected from halogen, CF₃, OH, C₁₋₆alkyl, and —OC₁₋₆alkyl;
each R^{b} is independently selected from the group:
1) CN,
2) —OH,
3) oxo,
4) halogen,
5) —C₁₋₆alkyl,
6) —O—C₁₋₆alkyl,
7) —C₃₋₆cycloalkyl,
8) —C₂₋₆cycloheteroalkyl,
9) aryl,
10) heteroaryl,
11) —C₁₋₆alkyl-aryl,
12) —C₁₋₆alkyl-heteroaryl,
13) —C₁₋₆alkyl-C₃₋₆cycloalkyl,
14) —C₁₋₆alkyl-C₂₋₆cycloheteroalkyl,
15) —(CH₂)_{q}—O—C₁₋₆alkyl,
16) —(CH₂)_{q}—O—C₃₋₆cycloalkyl,
17) —(CH₂)_{q}—O—C₂₋₆cycloheteroalkyl,
18) —(CH₂)_{q}—O-aryl,
19) —(CH₂)_{q}—O-heteroaryl,
20) —OC₁₋₆alkyl-C₃₋₆cycloalkyl,
21) —OC₁₋₆alkyl-C₂₋₆cycloheteroalkyl,
22) —OC₁₋₆alkyl-aryl,
23) —OC₁₋₆alkyl-heteroaryl,
24) —(CH₂)_{q}—S(O)ᵣR^{m},
25) —(CH₂)_{q}N(Rⁿ)₂,
26) —C(O)R^{o}, and
27) —C(O)NRⁿ,
wherein each R^{b} is unsubstituted or substituted with one to six substituents selected from halogen, CF₃, CF₂H, OCF₃, CN, CH₂CF₃, CF₂CH₃, —C₁₋₆alkyl, and —OC₁₋₆alkyl;
each R^{d} is independently selected from the group:
1) CF₃,
2) halogen, and
3) —C₁₋₆alkyl,
wherein alkyl is unsubstituted or substituted with one to three substituents selected from: CF₃, halogen, OH and —OC₁₋₆alkyl;
each R^{e} is independently selected from the group:
1) CF₃,
2) halogen, and
3) —C₁₋₆alkyl,
wherein alkyl is unsubstituted or substituted with one to three substituents selected from: CF₃, halogen, OH and —OC₁₋₆alkyl;
each R^{f} is independently selected from the group:
1) CF₃,
2) halogen, and
3) —C₁₋₆alkyl,
wherein alkyl is unsubstituted or substituted with one to three substituents selected from: CF₃, halogen, OH and —OC₁₋₆alkyl;
each R^{g} is independently selected from the group:
1) CF₃,
2) halogen, and
3) —C₁₋₆alkyl,
wherein alkyl is unsubstituted or substituted with one to three substituents selected from: CF₃, halogen, OH and —OC₁₋₆alkyl;
each R^{h} is independently selected from the group:
1) hydrogen,
2) —C₁₋₆alkyl,
3) —C₃₋₆cycloalkyl, and
4) —C₂₋₆cycloheteroalkyl,
wherein alkyl, cycloalkyl and cycloheteroalkyl is unsubstituted or substituted with one to three substituents selected from: —CF₃, halogen, OH and —OC₁₋₆alkyl;
each Rⁱ is independently selected from the group:
1) hydrogen,
2) —C₁₋₆alkyl,
3) —C₃₋₆cycloalkyl, and
4) —C₂₋₆cycloheteroalkyl,
wherein alkyl, cycloalkyl and cycloheteroalkyl is unsubstituted or substituted with one to three substituents selected from: CF₃, halogen, OH and —OC₁₋₆alkyl;
each R^{j} is independently selected from the group:
1) hydrogen,
2) —C₁₋₆alkyl,
3) —C₃₋₆cycloalkyl, and
4) —C₂₋₆cycloheteroalkyl,
wherein alkyl, cycloalkyl and cycloheteroalkyl is unsubstituted or substituted with one to three substituents selected from: CF₃, halogen, OH and —OC₁₋₆alkyl;
each R^{k} is independently selected from the group:
1) —C₁₋₆alkyl,
2) —C₃₋₆cycloalkyl, and
3) —C₂₋₆cycloheteroalkyl,
wherein alkyl, cycloalkyl, and cycloheteroalkyl is unsubstituted or substituted with one to three substituents selected from: —CF₃, halogen, OH and —OC₁₋₆alkyl;
each R^{L} is independently selected from the group:
1) hydrogen,
2) —C₁₋₆alkyl,
3) —C₃₋₆cycloalkyl, and
4) —C₂₋₆cycloheteroalkyl,
wherein alkyl, cycloalkyl, and cycloheteroalkyl is unsubstituted or substituted with one to three substituents selected from: —CF₃, halogen, OH and —OC₁₋₆alkyl;
each R^{m} is independently:
1) —C₁₋₆alkyl,
2) —C₃₋₆cycloalkyl, and
3) —C₂₋₆cycloheteroalkyl,
wherein alkyl, cycloalkyl and cycloheteroalkyl is unsubstituted or substituted with one to three substituents selected from: —CF₃, halogen, OH and —OC₁₋₆alkyl;
each Rⁿ is independently selected from the group:
1) hydrogen,
2) C₁₋₆alkyl,
3) —C₃₋₆cycloalkyl, and
4) —C₂₋₆cycloheteroalkyl,
wherein alkyl, cycloalkyl, and cycloheteroalkyl is unsubstituted or substituted with one to three substituents selected from: —CF₃, halogen, OH and —OC₁₋₆alkyl;
each R^{o} is independently selected from the group:
1) OH,
2) —C₁₋₆alkyl,
3) —C₃₋₆cycloalkyl, and
4) —C₂₋₆cycloheteroalkyl,
wherein alkyl, cycloalkyl and cycloheteroalkyl is unsubstituted or substituted with one to three substituents selected from —CF₃, halogen, OH and —OC₁₋₆alkyl;
r is 0, 1, 2, 3, 4, 5, or 6;
s is 0, 1, 2, 3, 4, 5, or 6;
t is 0, 1, 2, 3, 4, 5, or 6;
u is 0, 1, 2, 3, 4, 5, or 6;
p is 0, 1, 2, 3, 4, 5, or 6; and
q is 0, 1, 2, 3, 4, 5, or 6.

2. The compound according to claim 1 wherein W is CR⁶; or a pharmaceutically acceptable salt thereof.

3. The compound according to claim 1 wherein R¹ is C₂₋₁₂cycloheteroalkyl, wherein R¹ is unsubstituted or substituted with one to six substituents selected from R^{a}; or a pharmaceutically acceptable salt thereof.

4. The compound according to claim 1 wherein R¹ is selected from the group:
1) morpholine,
2) thiomorpholine,
3) piperidine,
4) piperazine,
5) pyrrolidine,
6) tetrahydropyran,
7) octahydro-1H-pyrrolo[2,3-c]pyridine,
8) 3-azabicyclo[3.1.0]hexane,
9) 5-azaspiro[2.4]heptane,
10) 1-oxa-7-azaspiro[4.4]nonane,
11) 1-oxa-8-azaspiro[4.5]decane,
12) 3-oxa-1,8-diazaspiro[4.5]decane,
13) 2,8-diazaspiro[4.5]decane,
14) 1-oxa-3,8-diazaspiro[4.5]decane,
15) 2-oxa-8-azaspiro[4.5]decane,
16) 1,8-diazaspiro[4.5]decane, and
17) 1-oxa-4,9-diazaspiro[5.5]undecane,
wherein R¹ is unsubstituted or substituted with one to six substituents selected from R^{a};
or a pharmaceutically acceptable salt thereof.

5. The compound according to claim 1 wherein R² is heteroaryl, wherein heteroaryl is unsubstituted or substituted with one to five substituents selected from R^{b}; or a pharmaceutically acceptable salt thereof.

6. The compound according to claim 1 wherein R² is aryl, wherein aryl is unsubstituted or substituted with one to five substituents selected from R^{b};
or a pharmaceutically acceptable salt thereof.

7. The compound according to claim 1 wherein R³ is selected from the group:
1) hydrogen, and
2) —C₁₋₆alkyl,
wherein R³ is unsubstituted or substituted with one to five substituents selected from R^{d};
or a pharmaceutically acceptable salt thereof.

8. The compound according to claim 1 wherein R³ is hydrogen; or a pharmaceutically acceptable salt thereof.

9. The compound according to claim 1 wherein R⁴ is selected from the group:
1) hydrogen,
2) OH,
3) CN,
4) CF₃,
5) CF₂H,
6) —C₁₋₆alkyl,
7) —O—C₁₋₆alkyl,
8) halogen, and
9) —C₁₋₆alkyl-O—C₁₋₆alkyl,
wherein R⁴ is unsubstituted or substituted with one to five substituents selected from R^{e};
or a pharmaceutically acceptable salt thereof.

10. The compound according to claim 1 wherein R⁴ is selected from the group:
1) hydrogen, and
2) —C₁₋₆alkyl,
wherein R⁴ is unsubstituted or substituted with one to five substituents selected from R^{e};
or a pharmaceutically acceptable salt thereof.

11. The compound according to claim 1 wherein R⁵ is selected from the group:
1) hydrogen,
2) OH,
3) CN,
4) CF₃,
5) CF₂H,
6) —C₁₋₆alkyl,
7) —O—C₁₋₆alkyl,
8) halogen, and
9) —C₁₋₆alkyl-O—C₁₋₆alkyl,
wherein R⁵ is unsubstituted or substituted with one to five substituents selected from R^{f};
or a pharmaceutically acceptable salt thereof.

12. The compound according to claim 1 wherein R⁵ is selected from the group:
1) hydrogen,
2) —C₁₋₆alkyl, and
3) halogen,
wherein R⁵ is unsubstituted or substituted with one to five substituents selected from R^{f};
or a pharmaceutically acceptable salt thereof.

13. The compound according to claim 1 wherein R⁶ is selected from the group:
1) hydrogen,
2) —C₁₋₆alkyl, and
3) —O—C₁₋₆alkyl,
wherein R⁶ is unsubstituted or substituted with one to five substituents selected from R^{g};
or a pharmaceutically acceptable salt thereof.

14. The compound according to claim 1 wherein R⁶ is hydrogen; or a pharmaceutically acceptable salt thereof.

15. The compound according to claim 1 wherein
R¹ is C₂₋₁₂cycloheteroalkyl, wherein R¹ is unsubstituted or substituted with one to six substituents selected from R^{a};
R² is heteroaryl, wherein R² is unsubstituted or substituted with one to five substituents selected from R^{b};
R³ is selected from the group:
1) hydrogen, and
2) —C₁₋₆alkyl,
wherein R³ is unsubstituted or substituted with one to five substituents selected from R^{d};
R⁴ is selected from the group:
1) hydrogen,
2) OH,
3) CN,
4) CF₃,
5) CF₂H,
6) —C₁₋₆alkyl,
7) —O—C₁₋₆alkyl,
8) halogen, and
9) —C₁₋₆alkyl-O—C₁₋₆alkyl,
wherein R⁴ is unsubstituted or substituted with one to five substituents selected from R^{e};
R⁵ is selected from the group:
1) hydrogen,
2) OH,
3) CN,
4) CF₃,
5) CF₂H,
6) —C₁₋₆alkyl,
7) —O—C₁₋₆alkyl,
8) halogen, and
9) —C₁₋₆alkyl-O—C₁₋₆alkyl,
wherein R⁵ is unsubstituted or substituted with one to five substituents selected from R^{f}; and
R⁶ is selected from the group:
1) hydrogen,
2) —C₁₋₆alkyl, and
3) —O—C₁₋₆alkyl,
wherein R⁶ is unsubstituted or substituted with one to five substituents selected from R^{g};
or a pharmaceutically acceptable salt thereof.

16. The compound according to claim 1 wherein
W is CR⁶;
R¹ is selected from the group:
1) morpholine,
2) thiomorpholine,
3) piperidine,
4) piperazine,
5) pyrrolidine,
6) tetrahydropyran,
7) octahydro-1H-pyrrolo[2,3-c]pyridine,
8) 3-azabicyclo[3.1.0]hexane,
9) 5-azaspiro[2.4]heptane,
10) 1-oxa-7-azaspiro[4.4]nonane,
11) 1-oxa-8-azaspiro[4.5]decane,
12) 3-oxa-1,8-diazaspiro[4.5]decane,
13) 2,8-diazaspiro[4.5]decane,
14) 1-oxa-3,8-diazaspiro[4.5]decane,
15) 2-oxa-8-azaspiro[4.5]decane,
16) 1,8-diazaspiro[4.5]decane, and
17) 1-oxa-4,9-diazaspiro[5.5]undecane,
wherein R¹ is unsubstituted or substituted with one to six substituents selected from R^{a};
R² is aryl, wherein aryl is unsubstituted or substituted with one to five substituents selected from R^{b};
R³ is hydrogen;
R⁴ is selected from the group:
1) hydrogen, and
2) —C₁₋₆alkyl,
wherein R⁴ is unsubstituted or substituted with one to five substituents selected from R^{e};
R⁵ is selected from the group:
1) hydrogen,
2) —C₁₋₆alkyl, and
3) halogen,
wherein R⁵ is unsubstituted or substituted with one to five substituents selected from R^{f}; and
R⁶ is hydrogen;
or a pharmaceutically acceptable salt thereof.

17. The compound according to claim 1 selected from:
1) (S)-1-(4-(2-hydroxy-4-(trifluoromethyl)phenyl)pyrido[3,4-d]pyridazin-1-yl)-3-methylpyrrolidin-3-ol;
2) (S)-1-(1-(2-hydroxy-4-(trifluoromethyl)phenyl)pyrido[3,4-d]-pyridazin-4-yl)-3-methylpyrrolidin-3-ol;
3) (cis)-4-(5-(2-(difluoromethoxy)-4-(trifluoromethyl)phenyl)pyrido[2,3-d]pyridazin-8-yl)-2,6-dimethylmorpholine;
4) 2-(4-((cis)-2,6-dimethylmorpholino)pyrido[3,4-d]pyridazin-1-yl)-5-(trifluoromethyl)phenol;
5) 2-(1-((cis)-2,6-dimethylmorpholino)pyrido[3,4-d]pyridazin-4-yl)-5-(trifluoromethyl)phenol;
6) 2-(5-((cis)-2,6-dimethylmorpholino)pyrido[2,3-d]pyridazin-8-yl)-5-(trifluoromethyl)phenol;
7) (cis)-4-(1-(2-(difluoromethoxy)-4-(trifluoromethyl)phenyl)pyrido[3,4-d]pyridazin-4-yl)-2,6-dimethylmorpholine;
8) 2-(8-((cis)-2,6-dimethylmorpholino)pyridazino[4,5-c]pyridazin-5-yl)-5-(trifluoromethyl)-phenol;
9) 2-(4-((cis)-2,6-dimethylmorpholino)pyridazino[4,5-d]pyridazin-1-yl)-5-(trifluoromethyl)-phenol;
10) (S)-1-(5-(2-hydroxy-4-(trifluoromethyl)phenyl)-2-methylpyrido[2,3-d]pyridazin-8-yl)-3-methylpyrrolidin-3-ol;
11) (cis)-4-(1-(benzofuran-5-yl)pyrido[3,4-d]pyridazin-4-yl)-2,6-dimethylmorpholine;
12) (S)-1-(5-(2-hydroxy-4-(trifluoromethyl)phenyl)-2-(trifluoromethyl)pyrido[2,3-d]pyridazin-8-yl)-3-methylpyrrolidin-3-ol;
13) (3S,4s,5R)-1-(4-(2-hydroxy-4-(trifluoromethyl)phenyl)pyridazino[4,5-d]pyridazin-1-yl)-3,4,5-trimethylpiperidin-4-ol;
14) 2-(4-((cis)-2,6-dimethylmorpholino)-5-methylphthalazin-1-yl)-5-(trifluoromethyl)phenol;
15) 2-(4-((cis)-2,6-dimethylmorpholino)-8-methylphthalazin-1-yl)-5-(trifluoromethyl)phenol;
16) (3S,4s,5R)-1-(1-(2-hydroxy-4-(trifluoromethyl)phenyl)pyrido[3,4-d]pyridazin-4-yl)-3,4,5-trimethylpiperidin-4-ol;
17) (3S,4s,5R)-1-(4-(2-hydroxy-4-(trifluoromethyl)phenyl)pyrido[3,4-d]pyridazin-1-yl)-3,4,5-trimethylpiperidin-4-ol;
18) (3S,4r,5R)-1-(1-(2-hydroxy-4-(trifluoromethyl)phenyl)pyrido[3,4-d]pyridazin-4-yl)-3,5-dimethylpiperidin-4-ol;
19) (3S,4r,5R)-1-(4-(2-hydroxy-4-(trifluoromethyl)phenyl)pyrido[3,4-d]pyridazin-1-yl)-3,5-dimethylpiperidin-4-ol;
20) 2-(4-((cis)-2,6-dimethylmorpholino)-6-methylphthalazin-1-yl)-5-(trifluoromethyl)phenol;
21) 2-(4-((cis)-2,6-dimethylmorpholino)-7-methylphthalazin-1-yl)-5-(trifluoromethyl)phenol;
22) (S)-1-(5-(2-hydroxy-4-(trifluoromethyl)phenyl)pyrido[2,3-d]-pyridazin-8-yl)-3-methylpyrrolidin-3-ol;
23) 2-(8-((3aS,7aR)-6-methyloctahydro-1H-pyrrolo[2,3-c]pyridin-1-yl)pyrido[2,3-d]pyridazin-5-yl)-5-(trifluoromethyl)phenol;
24) (3S,4s,5R)-1-(5-(2-hydroxy-4-(trifluoromethyl)phenyl)pyrido[2,3-d]pyridazin-8-yl)-3,4,5-trimethylpiperidin-4-ol;
25) 8-(5-(2-hydroxy-4-(trifluoromethyl)phenyl)pyrido[2,3-d]pyridazin-8-yl)-3-oxa-1,8-diazaspiro[4.5]decan-2-one;
26) (cis)-4-(5-(2-hydroxy-4-(trifluoromethyl)phenyl)pyrido[2,3-d]pyridazin-8-yl)-2,6-dimethylthiomorpholine 1,1-dioxide;
27) (R)-1-(5-(2-hydroxy-4-(trifluoromethyl)phenyl)pyrido[2,3-d]pyridazin-8-yl)-3-(trifluoromethyl)pyrrolidin-3-ol;
28) (S)-1-(5-(2-hydroxy-4-(trifluoromethyl)phenyl)pyrido[2,3-d]pyridazin-8-yl)-3-(trifluoromethyl)pyrrolidin-3-ol;
29) (R)-3-(difluoromethyl)-1-(5-(2-hydroxy-4-(trifluoromethyl)phenyl)pyrido[2,3-d]pyridazin-8-yl)pyrrolidin-3-ol;
30) (S)-3-(difluoromethyl)-1-(5-(2-hydroxy-4-(trifluoromethyl)phenyl)pyrido[2,3-d]pyridazin-8-yl)pyrrolidin-3-ol;
31) (R)-2-(8-(1-oxa-7-azaspiro[4.4]nonan-7-yl)pyrido[2,3-d]pyridazin-5-yl)-5-(trifluoromethyl)phenol;
32) (S)-2-(8-(1-oxa-7-azaspiro[4.4]nonan-7-yl)pyrido[2,3-d]pyridazin-5-yl)-5-(trifluoromethyl)phenol;
33) 2-(8-(1-oxa-8-azaspiro[4.5]decan-8-yl)pyrido[2,3-d]pyridazin-5-yl)-5-(trifluoromethyl)phenol;
34) 2-(8-(2-oxa-8-azaspiro[4.5]decan-8-yl)pyrido[2,3-d]pyridazin-5-yl)-5-(trifluoromethyl)phenol;
35) 9-(5-(2-hydroxy-4-(trifluoromethyl)phenyl)pyrido[2,3-d]pyridazin-8-yl)-1-oxa-4,9-diazaspiro[5.5]undecan-3-one;
36) 2-(8-((cis)-4,4-difluoro-3,5-dimethylpiperidin-1-yl)pyrido[2,3-d]pyridazin-5-yl)-5-(trifluoromethyl)phenol;
37) 1-(8-(5-(2-hydroxy-4-(trifluoromethyl)phenyl)pyrido[2,3-d]pyridazin-8-yl)-1,8-diazaspiro[4.5]decan-1-yl)ethan-1-one;
38) 1-(8-(5-(2-hydroxy-4-(trifluoromethyl)phenyl)pyrido[2,3-d]pyridazin-8-yl)-2,8-diazaspiro[4.5]decan-2-yl)ethan-1-one;
39) 8-(5-(2-hydroxy-4-(trifluoro-methyl)phenyl)pyrido[2,3-d]-pyridazin-8-yl)-3-methyl-1-oxa-3,8-diazaspiro[4.5]decan-2-one;
40) 2-(8-(4,4-dimethyl-1-oxa-8-azaspiro[4.5]decan-8-yl)pyrido[2,3-d]pyridazin-5-yl)-5-(trifluoromethyl)phenol;
41) (R)-1-(5-(2-hydroxy-4-(trifluoromethyl)phenyl)pyrido[2,3-d]pyridazin-8-yl)-3-phenylpyrrolidin-3-ol;
42) (S)-1-(5-(2-hydroxy-4-(trifluoromethyl)phenyl)pyrido[2,3-d]pyridazin-8-yl)-3-phenylpyrrolidin-3-ol;
43) 8-(5-(2-hydroxy-4-(trifluoromethyl)phenyl)pyrido[2,3-d]pyridazin-8-yl)-1-methyl-1,8-diazaspiro[4.5]decan-2-one;
44) 8-(5-(2-hydroxy-4-(trifluoromethyl)phenyl)pyrido[2,3-d]pyridazin-8-yl)-1-oxa-3,8-diazaspiro[4.5]decan-2-one;
45) 2-(8-(6,6-difluoro-3-azabicyclo[3.1.0]hexan-3-yl)pyrido[2,3-d]pyridazin-5-yl)-5-(trifluoromethyl)phenol;
46) 8-(5-(2-hydroxy-4-(trifluoromethyl)phenyl)pyrido[2,3-d]pyridazin-8-yl)-1-oxa-8-azaspiro[4.5]decan-2-one;
47) (R)-4,4-difluoro-1-(5-(2-hydroxy-4-(trifluoromethyl)phenyl)pyrido[2,3-d]pyridazin-8-yl)pyrrolidin-3-ol;
48) (S)-4,4-difluoro-1-(5-(2-hydroxy-4-(trifluoromethyl)phenyl)pyrido[2,3-d]pyridazin-8-yl)pyrrolidin-3-ol;
49) (R)-5-(5-(2-hydroxy-4-(trifluoromethyl)phenyl)pyrido[2,3-d]pyridazin-8-yl)-5-azaspiro[2.4]heptan-7-ol;
50) (S)-5-(5-(2-hydroxy-4-(trifluoromethyl)phenyl)pyrido[2,3-d]pyridazin-8-yl)-5-azaspiro[2.4]heptan-7-ol;
51) (R)-3-cyclopropyl-1-(5-(2-hydroxy-4-(trifluoromethyl)phenyl)pyrido[2,3-d]pyridazin-8-yl)pyrrolidin-3-ol;
52) (S)-3-cyclopropyl-1-(5-(2-hydroxy-4-(trifluoromethyl)phenyl)pyrido[2,3-d]pyridazin-8-yl)pyrrolidin-3-ol;
53) (R)-3-((dimethylamino)methyl)-1-(5-(2-hydroxy-4-(trifluoromethyl)phenyl)pyrido-[2,3-d]pyridazin-8-yl)pyrrolidin-3-ol;
54) (S)-3-((dimethylamino)methyl)-1-(5-(2-hydroxy-4-(trifluoromethyl)phenyl)pyrido-[2,3-d]pyridazin-8-yl)pyrrolidin-3-ol;
55) (R)-3-ethyl-1-(5-(2-hydroxy-4-(trifluoromethyl)phenyl)pyrido[2,3-d]pyridazin-8-yl)-pyrrolidin-3-ol;
56) (S)-3-ethyl-1-(5-(2-hydroxy-4-(trifluoromethyl)phenyl)pyrido[2,3-d]pyridazin-8-yl)-pyrrolidin-3-ol;
57) (R)-2-(8-(3-(hydroxymethyl)-3-methylpyrrolidin-1-yl)pyrido[2,3-d]pyridazin-5-yl)-5-(trifluoromethyl)phenol;
58) (S)-2-(8-(3-(hydroxymethyl)-3-methylpyrrolidin-1-yl)pyrido[2,3-d]pyridazin-5-yl)-5-(trifluoromethyl)phenol;
59) (R)-2-(8-(3-(2-hydroxypropan-2-yl)pyrrolidin-1-yl)pyrido[2,3-d]pyridazin-5-yl)-5-(trifluoromethyl)phenol;
60) (S)-2-(8-(3-(2-hydroxypropan-2-yl)pyrrolidin-1-yl)pyrido[2,3-d]pyridazin-5-yl)-5-(trifluoromethyl)phenol;
61) 2-(8-((2R,6R)-2-(hydroxymethyl)-6-methylmorpholino)pyrido[2,3-d]pyridazin-5-yl)-5-(trifluoromethyl)phenol;
62) 2-(8-((2S,6S)-2-(hydroxymethyl)-6-methylmorpholino)pyrido[2,3-d]pyridazin-5-yl)-5-(trifluoromethyl)phenol;
63) 2-(8-((2S,6S)-2-(hydroxymethyl)-6-methylmorpholino)pyrido[2,3-d]pyridazin-5-yl)-5-(trifluoromethyl)phenol;
64) 2-(8-((2R,6R)-2-(hydroxymethyl)-6-methylmorpholino)pyrido[2,3-d]pyridazin-5-yl)-5-(trifluoromethyl)phenol;
65) 1-((2R,4r,6S)-2,6-dimethyltetrahydro-2H-pyran-4-yl)-4-(2-methyl-4(trifluoromethyl)-phenyl)phthalazine;
66) (2R,4s,6S)-4-(5-(2-hydroxy-4-(trifluoromethyl)phenyl)pyrido[2,3-d]-pyridazin-8-yl)-2,6-dimethyltetrahydro-2H-pyran-4-ol;
67) (2R,4r,6S)-4-(5-(2-hydroxy-4-(trifluoromethyl)phenyl)pyrido[2,3-d]-pyridazin-8-yl)-2,6-dimethyltetrahydro-2H-pyran-4-ol;
68) (S)-1-(2-(difluoromethyl)-5-(2-hydroxy-4-(trifluoromethyl)phenyl)pyrido[2,3-d]pyridazin-8-yl)-3-methylpyrrolidin-3-ol; and
69) (S)-1-(3-(difluoromethyl)-5-(2-hydroxy-4-(trifluoromethyl)phenyl)pyrido[2,3-d]pyridazin-8-yl)-3-methylpyrrolidin-3-ol;
or a pharmaceutically acceptable salt thereof.

18. The compound according to claim 1 selected from:
1) (S)-1-(4-(2-hydroxy-4-(trifluoromethyl)phenyl)pyrido[3,4-d]pyridazin-1-yl)-3-methylpyrrolidin-3-ol;
2) (S)-1-(1-(2-hydroxy-4-(trifluoromethyl)phenyl)pyrido[3,4-d]-pyridazin-4-yl)-3-methylpyrrolidin-3-ol;
3) 2-(4-((cis)-2,6-dimethylmorpholino)pyridazino[4,5-d]pyridazin-1-yl)-5-(trifluoromethyl)phenol;
4) (S)-1-(5-(2-hydroxy-4-(trifluoromethyl)phenyl)pyrido[2,3-d]-pyridazin-8-yl)-3-methylpyrrolidin-3-ol;
5) 2-(8-((3aS,7aR)-6-methyloctahydro-1H-pyrrolo[2,3-c]pyridin-1-yl)pyrido[2,3-d]pyridazin-5-yl)-5-(trifluoromethyl)phenol;
6) (2R,4s,6S)-4-(5-(2-hydroxy-4-(trifluoromethyl)phenyl)pyrido[2,3-d]-pyridazin-8-yl)-2,6-dimethyltetrahydro-2H-pyran-4-ol; and
7) (2R,4r,6S)-4-(5-(2-hydroxy-4-(trifluoromethyl)phenyl)pyrido[2,3-d]-pyridazin-8-yl)-2,6-dimethyltetrahydro-2H-pyran-4-ol;
or pharmaceutically acceptable salts thereof.

19. A pharmaceutical composition comprising a compound of claim 1, or a pharmaceutically acceptable salt thereof, and a pharmaceutically acceptable carrier.

20-24. (canceled)

25. A compound according to claim 1, or a pharmaceutically acceptable salt thereof, for use in therapy.

26. A method of treating or preventing a disorder, condition or disease that is responsive to the inhibition of NLRP3 in a patient in need thereof comprising administration of a therapeutically effective amount of a compound according to claim 1, or a pharmaceutically acceptable salt thereof.

27. The method of claim 26 wherein the disorder is selected from: an inflammatory disorder, a fibrotic disorder, a cardiovascular disorder, a metabolic disorder and a neurodegenerative disorder.

28. The method of claim 27 wherein the disorder is an inflammatory disorder.

29. The method of claim 27 wherein the inflammatory disorder is selected from: an auto-immune disorder, an auto-inflammatory disorder, an inflammatory joint disorder, an inflammatory skin disorder, and a neuroinflammatory disorder.

30. The method of claim 26 wherein the disorder is selected from:
atherosclerosis, non-alcoholic steatohepatitis, Alzheimers disease and Parkinson's disease.

31. The compound according to claim 1 that is:
(S)-1-(4-(2-hydroxy-4-(trifluoromethyl)phenyl)pyrido[3,4-d]pyridazin-1-yl)-3-methylpyrrolidin-3-ol; or a pharmaceutically acceptable salt thereof.

32. The compound according to claim 1 that is: (S)-1-(1-(2-hydroxy-4-(trifluoromethyl)phenyl)pyrido[3,4-d]-pyridazin-4-yl)-3-methylpyrrolidin-3-ol; or a pharmaceutically acceptable salt thereof.

33. The compound according to claim 1 that is: 2-(4-((cis)-2,6-dimethylmorpholino)pyridazino[4,5-d]pyridazin-1-yl)-5-(trifluoromethyl)phenol; or a pharmaceutically acceptable salt thereof.

34. The compound according to claim 1 that is: (S)-1-(5-(2-hydroxy-4-(trifluoromethyl)phenyl)pyrido[2,3-d]-pyridazin-8-yl)-3-methylpyrrolidin-3-ol; or a pharmaceutically acceptable salt thereof.

35. The compound according to claim 1 that is: 2-(8-((3aS,7aR)-6-methyloctahydro-1H-pyrrolo[2,3-c]pyridin-1-yl)pyrido[2,3-d]pyridazin-5-yl)-5-(trifluoromethyl)phenol; or a pharmaceutically acceptable salt thereof.

36. The compound according to claim 1 that is: (2R,4s,6S)-4-(5-(2-hydroxy-4-(trifluoromethyl)phenyl)pyrido[2,3-d]-pyridazin-8-yl)-2,6-dimethyltetrahydro-2H-pyran-4-ol; or a pharmaceutically acceptable salt thereof.

37. The compound according to claim 1 that is: (2R,4r,6S)-4-(5-(2-hydroxy-4-(trifluoromethyl)phenyl)pyrido[2,3-d]-pyridazin-8-yl)-2,6-dimethyltetrahydro-2H-pyran-4-ol; or a pharmaceutically acceptable salt thereof.
